(19) United States
(12) Patent Application Publication
(10) Pub. No.: US 2024/0133870 A1
(43) Pub. Date: Apr. 25, 2024

(54) COMBINED TREATMENT FOR CANCER

(71) Applicant: Yeda Research and Development Co. Ltd., Rehovot (IL)

(72) Inventors: Ravid STRAUSSMAN, Mazkeret Batia (IL); Oded SANDLER, Rehovot (IL)

(73) Assignee: Yeda Research and Development Co. Ltd., Rehovot (IL)

(21) Appl. No.: 18/530,406

(22) Filed: Dec. 6, 2023

Related U.S. Application Data
(63) Continuation of application No. PCT/IL2022/050600, filed on Jun. 6, 2022.
(60) Provisional application No. 63/197,402, filed on Jun. 6, 2021.

(51) Int. Cl. G01N 33/50

(52) U.S. Cl. CPC G01N 33/5011; G01N 33/5088

(57) ABSTRACT

Combined treatment for cancer is provided. Accordingly, there is provided a method of selecting or determining therapeutic efficacy of a combination of agents for the treatment of cancer in a subject in need thereof, the method comprising: (i) culturing a cancerous tissue of the subject in an ex-vivo organ culture (EVOC) in the presence of a combination of an anti-cancer agent and an additional agent, said additional agent is inhibiting expression and/or activity of a target conferring innate resistance to said anti-cancer agent or increasing expression and/or activity of a target conferring innate sensitivity to said anti-cancer agent; and (ii) determining an anti-cancer effect of the combination on the tissue, wherein responsiveness of the tissue to the combination indicates the combination is efficacious for the treatment of the cancer in the subject. Also provided are methods of treating cancer with a combination of agent wherein cancerous tissue obtained from the subject demonstrates responsiveness to the combination of agents in an ex-vivo organ culture (EVOC).

### RELATED APPLICATIONS

This application is a Continuation of PCT Patent Application No. PCT/IL2022/050600 having International filing date of Jun. 6, 2022, which claims the benefit of priority under 35 USC § 119(e) of U.S. Provisional Patent Application No. 63/197,402 filed on Jun. 6, 2021. The contents of the above applications are all incorporated by reference as if fully set forth herein in their entirety.

### FIELD AND BACKGROUND OF THE INVENTION

The present invention, in some embodiments thereof, relates to combined treatment for cancer.

Advances in DNA sequencing and a fast growing arsenal of highly targeted anti-cancer drugs have made precision medicine possible for a growing number of cancer patients (1). Specific genetic alterations are frequently used as predictive biomarkers to stratify patients for treatments that match their tumor vulnerabilities. However, even though treatment is tailored to patient-specific genetic abnormalities, many patients demonstrate incomplete response to those drugs (2), thus remaining resistance to targeted therapy a major challenge in oncology. Resistance is mainly divided to early innate resistance (also known as also known as upfront or intrinsic resistance) and late acquired resistance, resulting from clonal evolution of resistant variants. Unlike the late emerging acquired resistance which results from selection of rare genetic alterations, the common innate drug resistance may stem in many cases from non-genetic alterations (3). Complex interactions with the tumor microenvironment (TME), such as the effect of TME secreted factors (secretome), have been shown to contribute to this type of resistance (e.g. 4-13 and Lippert et al. Arzneimittel-Forschung (Drug Research) (2008) 58(6): 261-264).

In stark contrast to the rapidly accelerating reliance on genetic profiling for precision medicine in cancer, profiling of potential mechanisms of innate resistance and integrating precision therapy with targeting of tumor-specific mechanisms of innate resistance are rarely integrated into the clinical decision-making process. Two of the reasons for that include the lack of knowledge of the potential mechanisms of resistance for the various drugs and cancer types; and no practical way in clinically relevant time scales to estimate, per patient, the relative contribution of each potential mechanism to drug resistance.

### SUMMARY OF THE INVENTION

According to an aspect of some embodiments of the present invention there is provided a method of selecting or determining therapeutic efficacy of a combination of agents for the treatment of cancer in a subject in need thereof, the method comprising:
(i) culturing a cancerous tissue of the subject in an ex-vivo organ culture (EVOC) in the presence of a combination of an anti-cancer agent and an additional agent, the additional agent is inhibiting expression and/or activity of a target conferring innate resistance to the anti-cancer agent or increasing expression and/or activity of a target conferring innate sensitivity to the anti-cancer agent; and
(ii) determining an anti-cancer effect of the combination on the tissue, wherein responsiveness of the tissue to the combination indicates the combination is efficacious for the treatment of the cancer in the subject.

According to an aspect of some embodiments of the present invention there is provided a method of treating cancer in a subject in need thereof, the method comprising:
(a) selecting treatment or determining therapeutic efficacy of a combination of agents according to the method; and
(b) administering to the subject a therapeutically effective amount of a combination demonstrating efficacy for the treatment of the cancer in the subject,
thereby treating the cancer in the subject.

According to some embodiments of the invention, the responsiveness is increased responsiveness as compared to individual treatment with the anti-cancer agent or the additional agent, as determined by the EVOC system.

According to some embodiments of the invention, the cancer is selected from the group consisting of melanoma, non-small cell lung cancer, ovarian cancer, breast cancer, pancreatic cancer, esophageal cancer, colorectal cancer and prostate cancer.

According to some embodiments of the invention, the cancer is selected from the group consisting of melanoma, colorectal cancer, non-small cell lung cancer and esophageal cancer.

According to some embodiments of the invention, cells of the cancer comprise a mutation associated with responsiveness to the anti-cancer agent.

According to some embodiments of the invention, the anti-cancer agent is a target therapy agent.

According to some embodiments of the invention, the anti-cancer agent is a cytotoxic agent.

According to some embodiments of the invention, the target has been identified in an in-vitro screening assay prior to the (i).

According to some embodiments of the invention, the target is a secreted factor or protein.

According to some embodiments of the invention, the cancer express a receptor of the target.

According to some embodiments of the invention, the additional agent binds a receptor of the target.

According to some embodiments of the invention, the target conferring innate resistance to the anti-cancer agent is selected from the group of targets listed in Table 3.

According to some embodiments of the invention, the target conferring innate resistance to the anti-cancer agent is selected from the group consisting of, epigen (EPGN), soluble epidermal growth factor receptor (EGFR), endothelial-monocyte activating polypeptide II (EMAPII), matrix metallopeptidase 7 (MMP7), neurotrophin4 (NTF4), lymphotoxin alpha (LTA), TNF superfamily member 14 (TNFSF14), bone morphogenetic protein 10 (BMP10), ciliary neurotrophic factor (CNTF), C—C motif chemokine ligand 1 (CCL1) and folate receptor beta (FOLR2).

According to some embodiments of the invention, the anti-cancer agent and the target conferring innate resistance to the anti-cancer agent are selected from the group of combinations listed in Table 4A.

According to some embodiments of the invention, the anti-cancer agent, the target conferring innate resistance to the anti-cancer agent and the cancer are selected from the group of combinations listed in Table 4A.

According to some embodiments of the invention, the cancer is a BRAF mutated melanoma cancer, the anti-cancer agent is a BRAF/MEK inhibitor and the target conferring innate resistance to the anti-cancer agent is selected from the group consisting of TGFA, HBEGF, NRG1b, HGF, FGF2, FGF9, EMAPII, FGF4, FGF6, FGF18, FGF7, LTA, TNF, IL1A, TGFB1, TGFB2, TGFB3 and OSM.

According to some embodiments of the invention, the cancer is a BRAF mutated melanoma cancer, the anti-cancer agent is a BRAF/MEK inhibitor and the additional agent is a MET inhibitor, EGFR inhibitor, HER2 inhibitor, TGFBR inhibitor, gp130 inhibitor, FGFR inhibitor and/or TNFR inhibitor.

According to some embodiments of the invention, the cancer is an EGFR mutated NSCLC cancer, the anti-cancer agent is an EGFR inhibitor and the target conferring innate resistance to the anti-cancer agent is selected from the group consisting of NRG1b, INS, HGF, FGF2, EMAPII and FGF4.

According to some embodiments of the invention, the cancer is an EGFR mutated NSCLC cancer, the anti-cancer agent is an EGFR inhibitor and the additional agent is a FGFR inhibitor, INSR inhibitor, FGFR inhibitor and/or MET inhibitor.

According to some embodiments of the invention, the cancer is an EGFR and PIK3CA mutated esophageal cancer, the anti-cancer agent is a PI3K inhibitor and the target conferring innate resistance to the anti-cancer agent is selected from the group consisting of EGF, BTC, TGFA, HBEGF, EPGN, NRG1a and NRG1b.

According to some embodiments of the invention, the cancer is an EGFR and PIK3CA mutated esophageal cancer, the anti-cancer agent is a PI3K inhibitor and the additional agent is a EGFR inhibitor, HER2 inhibitor, and/or HER3 inhibitor.

According to some embodiments of the invention, the target conferring innate sensitivity to the anti-cancer drug is selected from the group of targets listed in Table 5.

According to some embodiments of the invention, the target conferring innate sensitivity to the anti-cancer drug is selected from the group consisting of Transforming Growth Factor Beta 1-3 (TGFB1-3), Colony Stimulating Factor 2 (CSF2), Interleukin 10 (IL10), Platelet Derived Growth Factor Subunit B (PDGFB), Ephrin A5 (EFNA5), Soluble Epidermal Growth Factor Receptor (EGFR), Prokineticin 2 (PROK2), Relaxin 3 (RLN3), Peptide YY (PYY), acetylcholinesterase (ACHE), Amyloid P Component, Serum (APCS), Collagen Type IV Alpha 1 Chain (COL4A1) and Vitronectin (VTN).

According to some embodiments of the invention, the anti-cancer agent and the target conferring innate sensitivity to the anti-cancer drug are selected from the group of combinations listed in Table 6A.

According to some embodiments of the invention, the anti-cancer agent, the target conferring innate sensitivity to the anti-cancer drug and the cancer are selected from the group of combinations listed in Table 6A.

According to some embodiments of the invention, the cancer is a BRAF mutated melanoma cancer, the anti-cancer agent is a BRAF/MEK inhibitor and the target conferring innate sensitivity to the anti-cancer drug is selected from the group consisting of TGFB1, TGFB2, TGFB3, BMP2, CFS2,IL10, RLN3 and ACHE.

According to some embodiments of the invention, the cancer is an EGFR mutated NSCLC cancer or PDAC cancer, the anti-cancer agent is a mitosis inhibitor and the target conferring innate sensitivity to the anti-cancer drug is TGFB3 and/or BMP4.

According to some embodiments of the invention, the cancer is an ovarian cancer, the anti-cancer agent is an EGFR inhibitor and the target conferring innate sensitivity to the anti-cancer drug is TNFa.

According to some embodiments of the invention, the cancer is a BRAF wild-type melanoma, the anti-cancer agent is an MDM2 inhibitor or a Hsp90 inhibitor and the target conferring innate sensitivity to the anti-cancer drug is APCS.

According to an aspect of some embodiments of the present invention there is provided a method of treating cancer in a subject in need thereof, the method comprising administering to the subject a therapeutically effective amount of a combination of an anti-cancer agent and an additional agent inhibiting expression and/or activity of a target selected from the group consisting of epigen (EPGN), soluble epidermal growth factor receptor (EGFR), endothelial-monocyte activating polypeptide II (EMAPII), matrix metallopeptidase 7 (MMP7), neurotrophin4 (NTF4), lymphotoxin alpha (LTA), TNF superfamily member 14(TNFSF14), bone morphogenetic protein 10 (BMP10), ciliary neurotrophic factor (CNTF), C—C motif chemokine ligand 1 (CCL1) and folate receptor beta (FOLR2), wherein cancerous tissue obtained from the subject demonstrates responsiveness to the combination in an ex-vivo organ culture (EVOC), thereby treating the cancer in the subject.

According to some embodiments of the invention, the anti-cancer agent is selected from the group consisting of Mitosis inhibitor, DNA synthesis inhibitor, PI3K alpha inhibitor, BRAF/MEK inhibitor and EGFR inhibitor.

According to some embodiments of the invention, the cancer is selected from the group consisting of ovarian cancer, esophageal cancer, PDAC, BRAF wild-type melanoma, prostate cancer, breast cancer, BRAF mutated colorectal cancer, BRAF mutated melanoma and EGFR mutated NSCLC.

According to an aspect of some embodiments of the present invention there is provided a method of treating cancer in a subject in need thereof, the method comprising administering to the subject a therapeutically effective amount of a combination of an anti-cancer agent and an additional agent inhibiting expression and/or activity of a target, wherein the anti-cancer agent, the target and the cancer are selected from the group of combinations listed in Table 4B, and wherein cancerous tissue obtained from the subject demonstrates responsiveness to the combination of agents in an ex-vivo organ culture (EVOC), thereby treating the cancer in the subject.

According to an aspect of some embodiments of the present invention there is provided a method of treating cancer in a subject in need thereof, the method comprising administering to the subject a therapeutically effective amount of a combination of an anti-cancer agent and an additional agent increasing expression and/or activity of a target selected from the group consisting of Transforming Growth Factor Beta 1-3 (TGFB1-3), Colony Stimulating Factor 2 (CSF2), Interleukin 10 (IL10), Platelet Derived Growth Factor Subunit B (PDGFB), Ephrin A5 (EFNA5), soluble epidermal growth factor receptor (EGFR), Prokineticin 2 (PROK2), Relaxin 3 (RLN3), Peptide YY (PYY), acetylcholinesterase (ACHE), Amyloid P Component, Serum (APCS), Collagen Type IV Alpha 1 Chain (COL4A1) and Vitronectin (VTN), wherein cancerous tissue obtained from the subject demonstrates responsiveness to the combination in an ex-vivo organ culture (EVOC), thereby treating the cancer in the subject.

According to some embodiments of the invention, the anti-cancer agent is selected from the group consisting of BRAF/MEK inhibitor, EGFR inhibitor, HmG-CoA reductase inhibitor, Mdm2 inhibitor and Hsp90 inhibitor.

According to some embodiments of the invention, the cancer is selected from the group consisting of BRAF mutated melanoma, EGFR mutated NSCLC, PDAC and BRAF wild-type melanoma.

According to an aspect of some embodiments of the present invention there is provided a method of treating cancer in a subject in need thereof, the method comprising administering to the subject a therapeutically effective amount of a combination of an anti-cancer agent and an additional agent increasing expression and/or activity of a target, wherein the anti-cancer agent, the target and the cancer are selected from the group of combinations listed in Table 6B, and wherein cancerous tissue obtained from the subject demonstrates responsiveness to the combination of agents in an ex-vivo organ culture (EVOC), thereby treating the cancer in the subject.

According to an aspect of some embodiments of the present invention there is provided a method of treating cancer in a subject in need thereof, the method comprising administering to the subject a therapeutically effective amount of a combination of agents selected from the group of combinations listed in Table 7, wherein cancerous tissue obtained from the subject demonstrates responsiveness to the combination of agents in an ex-vivo organ culture (EVOC), thereby treating the cancer in the subject.

According to some embodiments of the invention, the cancer is selected from the group consisting of BRAF mutated melanoma, EGFR mutated NSCLC, PDAC, ovarian cancer, esophageal cancer, prostate cancer, breast cancer, BRAF mutated colorectal cancer and BRAF wild-type melanoma.

According to an aspect of some embodiments of the present invention there is provided a method of treating cancer in a subject in need thereof, the method comprising administering to the subject a therapeutically effective amount of a combination of agents, wherein the combination of agents and the cancer are selected from the group of combinations listed in Table 8, and wherein cancerous tissue obtained from the subject demonstrates responsiveness to the combination of agents in an ex-vivo organ culture (EVOC), thereby treating the cancer in the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEW OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-H demonstrate the identified landscape of tumor secretome-mediated innate drug resistance. FIG. 1A shows the main categories of the 321 factors used in the secretome screen.

FIG. 1B shows the timeline of the in-vitro secretome screen. FIG. 1C shows growth curves of GFP-positive cancer cell lines demonstrating the effect of drugs with or without specific secreted factors on the total GFP count as a proxy for the number of cells. AZD6244—MEK inhibitor (4 M), FGF7 (160 ng/ml); neratinib—EGFR inhibitor (0.25 μM), PRL—prolactin (125 ng/ml). P-value of the fold change in GFP level at day 7 relative to control was calculated by two-sided t-test. * p<0.05, *** p<0.001. Average rScore (+/−SE) was calculated from three different experiments. FIG. 1D shows representative images in GFP channel from day 7 of the experiments. Scale bar represents 200 μm. FIG. 1E is a summary table of drug resistance mediating factors. Screens were grouped by cancer type and drug targets. The number of screens per group (n) is shown at the bottom of each column. The effect of each secreted factor on the cell lines in each group was collapsed into four ranks, as shown in FIGS. 9A-D. FIG. 1F shows the effect of secreted factors on the response of the G361 μmelanoma cell line to vemurafenib in 2D and 3D culturing systems. Day 7-Day 1 GFP reads per cytokine were converted into z-scores. Z-score values were averaged over two independent experiments. All factors with z-score>1 are represented on the scatter plot. Pearson correlation coefficient is 0.79. FIG. 1G shows unsupervised hierarchical clustering (Euclidean distance) of 185 BRAF V600E melanoma patients from TCGA by AXL/MITF gene signatures of sensitivity or resistance to BRAF/MEK inhibition. Clusters with most differential signature expression patterns were selected for further analysis. A vertical dashed line defines resistant patients cluster (left, N=26) and sensitive patients cluster (right, N=67). FIG. 1H shows expression difference of resistance-mediating factors between the two clusters of melanoma patients presented in FIG. 1G. Z-scores of the expression across all 185 patients were calculated for each of the factors (excluding factors with expression level below the 25th percentile of whole genome expression: FGF4, FGF6, INS) that were found to mediate resistance to melanoma BRAF-mutated cell lines from BRAF/MEK inhibition. Delta of mean z-score between the resistant cluster and sensitive cluster is shown. ** P-value<0.01 by Monte Carlo simulation for obtaining a similar expression trend with a similar number of random genes.

FIGS. 2A-G demonstrate that EMAPII may mediate resistance of melanoma cells to BRAF/MEK inhibition by FGFR signaling. FIGS. 2A-B show growth curves of GFP-positive BRAF (V600E) melanoma cell lines demonstrating the effect of EMAPII on the sensitivity to BRAF/MEK inhibition. PLX4720—BRAFi, 2 μM; PD184352—MEKi, 1 μM, EMAPII—50 ng/ml. P-value of the difference between GFP(drug)/GFP(no-treatment) and GFP(drug+EMAPII)/GFP(no-treatment), at day 7, was calculated by two-sided t-test. ** p<0.01, *** p<0.001. Average rScore (+/−SE) was calculated from at least four different experiments. FIG. 2C is a scatter plot demonstrating the correlation between FGF2 and EMAPII effects on the sensitivity of 22 BRAF (V600E) melanoma cell lines to BRAF/MEK inhibition. A total of 69 experiments are shown. FIG. 2D shows correlation matrix between the rScores of 14 resistance-mediating factors (ranked 3) across 22 BRAF (V600E) melanoma cell lines. FIG. 2E demonstrates that FGFR inhibitor abrogates EMAPII/AIMP1-mediated resistance. The effect of FGF2 (10 ng/ml), EMAPII (10 ng/ml) or AIMP1 (10 ng/ml) on G361 μmelanoma cells treated with vemurafenib (2 μM) was measured with or without the FGFR inhibitor PD17307 (0.5 μM). Data represent an average of 4 to 8 experiments. P-value was calculated by two-sided t-test. *** p<0.001. Error bars represent standard error. FIG. 2F demonstrates the effect of knocking down putative AIMP1 receptors on AIMP1-mediated resistance. 18 putative AIMP1 receptors were knocked down by shRNAs (on average 6.8 shRNAs/gene) in the G361 cell line. shRNAs toward luciferase served as a negative control. The effect of AIMP1 (50 ng/ml) on the sensitivity to PLX4720 (2 μM) was tested in all knockdown clones and normalized to no-shRNA control. Results represent the average of 3-8 experiments. Error bars represent standard error. P-values were calculated by two-sided t-test relative to luciferase. * p<0.05, Q-value=0.1. FIG. 2G shows in-cell Western of pERK reactivation by EMAPII (200 ng/ml) or FGF2 (200 ng/ml) in G361 cell line treated with vemurafenib (2 μM) and trametinib (1 nM). pERK levels were normalized to the total number of cells in each well, as measured by DRAQ5 and to no drug (DMSO) control. The average of 4 experiments is presented. Error bars represent standard error. P-value was calculated by two-sided t-test. *** p<0.001.

FIGS. 3A-L demonstrate that inter-cancer variability in the effects of secreted factors on the sensitivity to drugs may stem from differences in expression of the corresponding receptors. FIGS. 3A, 3B, 3E, 3F, 3I and 3J show growth curves of GFP-positive cancer cell lines, demonstrating the factors-specific effect on the sensitivity to drugs. PD184352 (MEKi, 1 μM), AZD6244 (MEKi, 4 μM), PLX4720 (BRAFi, 2 μM), afatinib (EGFR/HER2i, 0.1 μM), lapatinib (EGFR/HER2i, 2 μM), BTC (EGFR ligand beta-cellulin, 100 ng/ml), FGF10 (FGFR2 μligand FGF10, 100 ng/ml) NRG1a (HER2/3, HER2/4 μligand neuregulinl-alpha, 50 ng/ml). P-values were calculated based on two-sided proportion test between all cell lines in each cancer type treated with the designated drugs. The proportion was calculated by the number of experiments with rScore>0.2 divided by the total number of experiments. *** P<0.001. FIGS. 3C, 3G and 3K show box plots of the expression of the relevant receptors in cell lines from the CCLE database representing the various cancer types. P-value was calculated by two-sided Mann-Whitney test. *** P<0.001. FIGS. 3D, 3H and 3L show box plots of the expression of the relevant receptors in human tumors from the TCGA database, representing the different cancer types. P-value was calculated by two-sided Mann-Whitney test. ** P<0.01, *** P<0.001.

FIGS. 4A-C demonstrate tissue-specific effects on innate drug resistance. FIG. 4A demonstrates that tissue-specific stromal cells may induce different innate resistance mechanisms. SK-MEL-5 BRAF (V600E) melanoma cells were co-cultured with the lung-derived stromal cell line WI-38 or with the bone marrow-derived stromal cell line HS-5 with or without vemurafenib (4 μM). To try to abrogate the observed stromal-mediated resistance to vemurafenib, six drugs that target the main mechanisms of resistance to BRAF inhibition (FIG. 1E) were added to the culture. Vehicle—DMSO, EGFRi—gefitinib (0.1 μM), EGFRi/HER2i—lapatinib (10 nM), METi—crizotinib (0.1 μM), FGFRi—AZD4547 (50 nM), NFkB/TNFRi—CAPE (10 μM), TGFBRi-LY2109761 (0.5 μM), gp130i—SC144 (1 nM). P-values were calculated by two-sided t-test. *** P<0.001. Error bars represent standard error. FIG. 4B shows levels of HGF and FGF2 from pre-conditioned media of WI-38 and HS-5 cell lines measured by ELISA. P-values were calculated by two-sided t-test. *** P<0.001. Error bars represent standard error. FIG. 4C shows tissue-specific effect on pERK inhibition by vemurafenib. Human BRAF (V600E) melanoma cell lines UACC62 or G361 were used to generate xenograft tumor models in various tissues of nude mice. When tumors reached a volume of 500-700 μmm³, they were resected, sliced, and cultured ex-vivo in the presence of different drug combinations or DMSO control. Following 4 days of drug treatment, slices were fixed, embedded in paraffin blocks, and subjected to immunohistochemistry, using anti-pERK antibody, or to immuno-fluorescence, using anti-pFGFR1 antibody. BRAFi (vemurafenib, 4 μM), FGFRi (AZD4547, 2 μM). Scale bar represents 50 μm.

FIGS. 5A-D demonstrate that multiple layers of complexity impede the clinical implementation of co-targeting innate mechanisms of drug resistance. FIG. 5A is a scatter plot depicting the variability of the expression of 17,281 genes across 473 TCGA human melanoma tumors vs. their median expression level. Expression variability is represented by quartile-based coefficient of variation (QCV), calculated as (forth quartile—first quartile)/median. Each gene is represented by a blue dot. Black dots represent median QCV values of bins of 250 genes. Resistance-mediating factors in BRAF (V600E) melanoma cell lines are represented by red dots, and their corresponding receptors are represented by orange squares. Both receptors and factors are significantly enriched in the group of genes with QCV above median (P-value<0.01 by hypergeometric test). FIG. 5B shows box plots demonstrating the distribution of the expression of syndicans and glypicans among 145 TCGA BRAF (V600E) melanoma patients. Boxes extend from 25th to 75th percentiles; line in the middle of the box represents the median. Error bars are drawn down to the 5th percentile and up to the 95th percentile. FIG. 5C shows representative graphs demonstrating the change in expression of five factors that can mediate resistance to BRAF/MEK inhibition of melanoma cell lines. 19 BRAF (V600E) human melanoma tumors were biopsied pre-treatment as well as 3-8 weeks on treatment with BRAF inhibitors and subjected to RNA-sequencing. FIG. 5D is a heat map demonstrating the effect of factors on the sensitivity of 22 BRAF (V600E) melanoma cell lines to BRAF/MEK inhibition. Effect is quantified by rScore. Only factors with strong effect on melanoma cell lines (FIG. 1E, ranks 2-3) are included. The number of drugs that are needed to overcome all potential resistance mechanisms for each of the cell lines is shown below the heatmap.

FIGS. 6A-G demonstrate implementation of integrative precision therapy for improving treatment efficacy in BRAF (V600E) cancer models. FIG. 6A is a bar graph demonstrating the ex-vivo viability of UACC62 xenograft subcutaneous tumors under different drug combinations. 500-700 μmm³ tumors were resected, sliced, and cultured ex-vivo. Following 4 days of drug treatment, slices were fixed and embedded in paraffin blocks. FFPE slices were stained by H&E, and the percentage of viable cancer cells was morphologically assessed on H&E stained sections by a pathologist as the ratio between viable cancer cell area and total cancer area (viable cancer cells plus necrotic cancer cells). Vehicle—DMSO, METi—crizotinib 2 μM, EGFRi—gefitinib 2 M, EGFR/HER2i—lapatinib 2 μM, TGFBRi—LY2109761 2 μM, gp130i—SC144 2 μM, FGFRi—AZD4547 2 μM, TNFRi—R-7050 2-5 μM, BRAFi/MEKi—vemurafenib 4 μM/trametinib 0.5 μM. Each black dot in a given treatment condition represents a different tumor. P-values were calculated by two-sided t-test. *** P<0.001 ** P<0.01. Error bars represent standard error. FIG. 6B demonstrates the effect of 297 secreted factors on the sensitivity of UACC62 BRAF (V600E) melanoma cell line to BRAF/MEK inhibition. rScores were sorted from high to low. Names of the six secreted factors with the highest rScore are shown. Factors related to TNF pathways are marked in gray. Results represent the average of at least two experiments. FIG. 6C shows representative images from FIG. 6A. Viability percentage is given per treatment combination. Scale bar represents 50 μm. FIG. 6D demonstrates the results of an in-vivo preclinical experiment with UACC62 bearing mice. Vehicle—18% DMSO, 22% PEG300, 4% TWEEN 80. FGFRi/TNFRi—AZD4547 12.5 μmg/Kg, R7050 15 μmg/kg i.p, daily. BRAFi—vemurafenib 25 mg/kg, i.p, twice a day. Cohort size pre-treatment: 5-6, P-values of the difference on the last day of experiment were calculated by one-sided Mann-Whitney test. * P<0.05. Error bars represent standard error. FIG. 6E demonstrate results of an EVOC experiment of a melanoma patient. Patient responded temporarily to BRAF/MEK and was non-responsive to immune checkpoint inhibitors. Shown are tumor slices treated with BRAFi/MEKi with or without the addition of TNFRi/FGFRi, two tumor sites per treatment. From each tumor site, enlarged areas (marked in red squares) are presented. Drugs and concentrations are similar to FIG. 6A. Viability percentage was calculated based on the entire field of view per treatment combination. Black scale bar represents 500 μm. Blue scale bar represents 50 μm. FIG. 6F shows representative images of ex-vivo viability of HT-29 orthotopic model under different drug combinations. Tumors from the colon sub-mucosa were resected, sliced, and cultured ex-vivo. Following 4 days of drug treatment, slices were fixed and embedded in paraffin blocks. FFPE slices were stained by pERK and pERK activity was assessed by a pathologist. Vehicle—DMSO, FGFRi—AZD4547 2 μM, EGFR/HER2i—lapatinib 4 μM, BRAFi-vemurafenib 4 μM. Scale bar represents 50 μm. FIG. 6G shows immunofluorescence images of pHER3 and the corresponding ligand NRG1 of vehicle and BRAFi blocks from FIG. 6F. Scale bar represents 50 μm.

FIGS. 7A-D demonstrate implementation of integrative precision therapy for improving treatment efficacy in EGFR-mutated NSCLC models. FIG. 7A demonstrates the results of EVOC experiments of H1975 xenografts with different drug combinations. Vehicle—DMSO, FGFRi—AZD4547 2-4 μM, INSRi—linsitinib 2 μM, METi—crizotinib 2-4 μM, EGFRi—afatinib 3-4 μM. Each black dot in a given treatment condition represents a different tumor. P-values were calculated by two-sided t-test. ** P<0.01, * P<0.05. Error bars represent standard error. FIG. 7B shows representative images from FIG. 7A. Viability percentage is given per treatment combination. Scale bar represents 50 μm. FIG. 7C demonstrates the results of an in-vivo preclinical experiment with H1975-bearing mice: Vehicle—3.2% DMSO, 25% PEG300, 4% TWEEN 80. EGFRi—afatinib 20 μmg/kg, FGFRi-AZD4547 10 μmg/kg, INSRi—linsitinib-20 mg/kg. All drug combinations were administered per os, daily. Cohort size pre-treatment: 4-5, P-values of the difference on the last day of experiment were calculated by one-sided Mann-Whitney test. ** P<0.01, * P<0.05. Error bars represent standard error. FIG. 7D demonstrates the results of an EVOC experiment of a NSCLC patient. Non-smoker female with adenocarcinoma (EGFR mutation: p.Leu747_Ala750del insPro). EGFRi—gefitinib 1.6 μM, FGFRi—AZD4547 0.2 μM. The percentage of viable cancer cells was morphologically assessed on H&E stained sections by a pathologist as the ratio between viable cancer cell area and total cancer area (viable cancer cells plus necrotic cancer cells). The most viable region is presented per treatment combination. Viability score was calculated based on the entire field of view. Scale bar represents 50 μm.

FIGS. 8A-D show schematic overviews of rScore and bScore calculation. Calculation of rScore and bScore is derived from the data points A, B, C, D marked on GFP-level curves of cells under different treatment conditions, as shown in FIGS. 8A-B. As all cells used in the screen are constitutively expressing GFP, GFP level was used as a proxy for the number of cells. FIG. 8A demonstrates the calculation of pScores and rScores. Proliferation score (pScore) describes the percent of change in the number of cells after seven days of treatment with a given factor. Rescue score (rScore) quantifies the effect of a given factor on the resistance to a given drug. Relative rescue describes the proportion of cells treated with drug+factor out of untreated cells. The higher this ratio, the stronger effect the factor has on drug resistance. Residual drug growth describes the proportion of cells growth under drug relative to no treatment condition. The higher this ratio, the less effective is the drug to start with. Finally, the rescue score (rScore) is the relative rescue after adding a penalty for drugs with low efficacy. FIG. 8B demonstrates the calculation of bScores. Bliss Score (bScore) quantifies the extent of synergism between a given factor and a given drug. Factor effect describes the proportion (or probability) of cells killed by the factor. Drug effect describes the proportion (or probability) of cells killed by the drug. The observed killing effect (observed effect) describes the proportion (or probability) of cells killed by both factor and drug. Assuming drug killing and factor killing are independent events, the expected killing effect (expected effect) is the sum of probabilities D and F excluding events intersection (D*F), which represents a subpopulation of cells that may be sensitive to both the drug and factor. Finally, bliss score (bScore) is the delta between observed and expected killing multiplied by −1 to symbolize the expected decrease in the number of cancer cells following the addition of a factor. FIG. 8C shows nine examples of possible factor effects on cells proliferation and response to drugs. Upper panel—effect on cells proliferation. Of note, this effect may be independent of the factor's effect on drug resistance (rScore) or synergism (bScore). Middle panel—effect on cell resistance to drug. Note that on the right panel in this row, while the factor abrogates most of the drug effect, the rScore is <0.2 due to the penalty for the relatively low drug efficacy. Indeed, the rScore calculator was designed to give higher scores to cases in which factors may have a significant clinically-relevant effects on the response to therapy. Cases in which the drug exhibits a very low efficacy even without the presence of a factor are of less clinical relevance. A threshold for a strong factor's effect on the resistance to drug was set at rScore>0.2. Lower panel—effect on drug synergism. A threshold for considering a drug-factor interaction as synergistic was set at bScore<−0.15. FIG. 8D is a table of factors with large effects on cell proliferation. In 79 control experiments, the effect of all 321 factors was tested on cell lines with DMSO rather than with a drug. The number of experiments (out of 79) with pro-proliferative effect (pScore>30%) and anti-proliferative effect (pScore<−30%) were counted for each factor in the secretome library. Factors were sorted by delta count of pro-proliferative and anti-proliferative experiments. Top positive and negative factors are presented. Reassuringly, the results demonstrate factors with known pro- and anti-proliferative effects.

FIGS. 9A-D demonstrate rank calculation of the effect of each factor in a given group of experiment (e.g. all BRAF (V600E)-mutated melanoma cell lines treated with BRAF or BRAF/MEK inhibitors). The rank calculator was designed to capture both the factor's effect on outlier experiments (the tail of the distribution) as well as the factor's effect on the general trend in the entire group of experiments (the center of the distribution). The rank was determined by the sum of rewards given by examining the tail and the center of the rScores distribution. FIG. 9A is a flow chart demonstrating calculation of rank by rScore values. The higher the rScore, the stronger the factor effect on resistance to a drug. Reward given according to the tail of the distribution was determined by an rScore threshold (rScore≥0.2) or by distance from the rScore mean of the entire group of experiments (rScore≥group mean rScore+2 standard deviations). Reward given according to the center of the distribution was determined by the distance of the rScore median of the entire group of experiments, from the distribution center (median rScore≥mean rScore+1 standard deviation). FIG. 1B shows four examples of rScore distributions. The rScore distributions of 4 factors across 80 experiments of BRAF (V600E) mutated melanoma cell lines treated with BRAF/MEK inhibitors are shown. Following the chart in FIG. 9A, the ranking calculation of FGF2 is as follows: FGF2 rScore values are above 0.2 (thick black dashed line) in multiple experiments, for which it was rewarded +2. FGF2 rScore values are above the group mean rScore+2 standard deviations (thick blue dashed line) in multiple experiments, for which it was rewarded +1. Thus, the sum of rewards of FGF2 has reached 3, which sets its rank on 3. FIG. 9C is a flow chart demonstrating the calculation of rank by bScore values. The lower the bScore, the stronger the factor's synergism with the drug. Reward given according to the tail of the distribution was determined by a bScore threshold (bScore<=−0.15) or by distance from the bScore mean of the entire group of experiments (bScore<=group mean bScore—2 standard deviations). Reward given according to the center of the distribution was determined by the distance of the bScore median of the entire group of experiments, or the distance of a single experiment from the distribution center (bScore≤mean bScore−1 standard deviation). FIG. 9D shows four examples of bScore distributions. The bScore distributions of 4 factors across 80 experiments of BRAF (V600E) mutated melanoma cell lines treated with BRAF/MEK inhibitors are shown. Following the chart in FIG. 9C, the ranking calculation of TGFB3 is as follows: TGFB3 bScore values are below −0.15 (thick black dashed line) in multiple experiments, for which it was rewarded +2. TGFB3 bScore values are below the group mean bScore—2 standard deviations (thick blue dashed line) in multiple experiments, for which it was rewarded +1. Thus, the sum of rewards of TGFB3 has reached 3, which sets its rank on −3.

FIGS. 10A-D demonstrate the identified landscape of tumor secretome-mediated innate drug synergism. FIG. 10A is a summary table of factors that were found to have a synergistic activity when given with drugs. Screens were grouped by cancer type and drug targets. The number of screens per group (n), is shown at the bottom of each column. The effect of each secreted factor on the cell lines in each group was collapsed into four ranks as described in FIGS. 9A-D. FIGS. 10A-B show examples of growth curves of GFP-positive melanoma BRAF (V600E) melanoma cell lines demonstrating the synergistic effect of drugs with or without acetylcholinesterase (ACHE) on the total GFP count as a proxy for the number of cells. PD184352—MEK inhibitor (1 μM). For each growth curve, bScore is indicated.

FIGS. 11A-D demonstrate the effect of TNF pathway on the resistance of melanoma cells to BRAF/MEK inhibition. FIGS. 11A-B show that expression of the indicated TNF receptors in human biopsies from melanoma tumors of treatment-naïve melanoma patients correlates with stronger initial response to BRAF/MEK inhibition. This observation is in line with FIG. 1H, which demonstrated that the expression of all resistance mediating factors but TNFR ligands is associated with a gene signature of resistance to BRAFi. Data is composed from two data sets: Blue—Van Ellen (PMID: 24265154, n=8), Red—Kwong dataset (PMID: 25705882, n=14). Pearson correlation was calculated per data set. The Combined P-value (Fisher method) for both correlations (TNFRSFlA and TNFRSFlB) is <0.05. FIGS. 11C—demonstrate TNF effect on the expression signature for resistance to BRAFi (Konieczkowski et al., PMID: 24771846) in human melanoma BRAF (V600E) cell lines. Expression of drug resistance markers (AXL, TPM1) and drug sensitivity markers (MITF, MLANA, PMEL, TYRP) was measured by RT-qPCR pre- and 24 hours post incubation with 25 ng/ml TNFa. The ratio between signature gene expression post-incubation and pre-incubation was normalized to no treatment control, yielding the relative fold change. Black whiskers represent error bars from two biological repeats, each done in triplicates. While in the UACC62 cell line (FIG. 11D) TNFa shifts the expression of the gene signature from BRAFi sensitive mode to BRAFi resistant mode, the expression of this gene signature shows smaller changes in the MALME-3M cell line (FIG. 11C). Of note, TNF ligands were found to confer resistance to BRAFi/MEKi in UACC62 but not in MALME-3M cell line.

FIG. 12 demonstrates the relative expression of FGFR2IIIb in colorectal vs melanoma BRAF (V600E) cell lines. Shown the expression fold change relative to Adenine Phosphoribosyltransferase (APRT) in CRC BRAF mutated cell line (HT-29) as compared to melanoma BRAF mutated cell lines (UACC62, SKMEL5). Error bars represent extreme values.

FIGS. 13A-B demonstrate EVOC timeline and the effect on tissue viability. FIG. 13A is a schematic presentation of the EVOC pipeline. Following tumor resection, tissue is cut into ˜250 m thick slices. Slices are then placed on a mesh and incubated for 96 hours in 37° C. and 80% oxygen, during which each slice may be treated with different drugs. Finally, slices are fixed and can be used for multiple IHC stainings. FIG. 13B shows immunohistochemistry of HT-29 colon xenograft either untreated (left) or treated with AZD4547 0.5 μM. H&E and BrDu staining following 96 hours of incubation show live and proliferating tissue.

FIGS. 14A-J demonstrate the variability in the expression of resistance mediating factors and their corresponding receptors among cancer patients. FIG. 14A-B demonstrate the variation in RNA expression of factors mediating innate resistance to BRAF/MEK inhibitors in melanoma BRAF (V600E) cell lines (FIG. 14A) or their corresponding receptors (FIG. 14B) among melanoma BRAF (V600E) mutated patients from TCGA (n=145). Boxes extend from 25^{th} to 75^{th} percentiles, the line in the middle of the box represents the median, whiskers are drawn down to the 5^{th} percentile and up to the 95^{th} percentile. Lower panels: expression pattern from regional lymph node melanoma of three patients demonstrating the patient-specific pattern of expression. Values in the lower panels are given in log scale and floored or ceiled to 1 or 4, respectively. FIG. 14C shows quantification of the signal obtained from tumor microarrays (TMA) of melanoma BRAF (V600E) tumors from treatment-naïve patients subjected to multiplexed immunofluorescence (IF) staining of factors that can mediate resistance to BRAF/MEK inhibition. Each factor's fluorescent read of the entire patient biopsy was normalized to its mean fluorescence across 28-34 patients (see methods). Median—blue line. inter-quartiles—black whiskers. FIG. 14D are staining images of selected patients from FIG. 14C. Scale bar is 100 μm. Upper row—H&E staining. Second row—PanMel staining for detecting melanoma cells in the sections. FIGS. 14E-G demonstrate the variation in RNA expression of factors mediating innate resistance to EGFR/HER2 inhibitor in Breast HER2 amplified cell lines (FIG. 14E) or their corresponding receptors (FIG. 14F) among Breast HER2 amplified patients from TCGA (n=286). Boxes extend from 25^{th} to 75^{th} percentiles, the line in the middle of the box represents the median, whiskers are drawn down to the 5^{th} percentile and up to the 95^{th} percentile. Lower panels: expression pattern from breast primary tumor of three patients demonstrating the patient-specific pattern of expression. Values in the lower panels are given in log scale and floored or ceiled to 1 or 3, respectively. FIG. 14G is a scatter plot depicting the variability of the expression of 17,673 genes across 1215 TCGA human breast tumors vs. their median expression level. Expression variability is represented by quartile based coefficient of variation (QCV) calculated as (forth quartile—first quartile)/median. Each gene is represented by a blue dot. Black dots represent median QCV values of bins of 250 genes. Resistance mediating factors in HER2 amplified breast cell lines are represented by red dots while their corresponding receptors are represented by orange squares. Both receptors and factors are significantly enriched in the group of genes with QCV above median (P-value<0.001 by hypergeometric test). FIG. 14H-J demonstrate the variation in RNA expression of factors mediating innate resistance to EGFR inhibitors in NSCLC EGFR-mutated cell lines (FIG. 14H) or their corresponding receptors (FIG. 141) among EGFR-mutated patients from GSE31210 (n=127). Boxes extend from 25^{th} to 75^{th} percentiles, the line in the middle of the box represents the median, whiskers are drawn down to the 5^{th} percentile and up to the 95^{th} percentile. Lower panels: expression pattern from NSCLC primary tumor of three patients demonstrating the patient-specific pattern of expression. Values in the lower panels are given in log scale and floored or ceiled to 1 or 3, respectively. FIG. 14J is a scatter plot depicting the variability of the 54675 gene probes, expression representing 23344 genes, across 246 NSCLC patients from GSE31210 vs. their median expression level. Expression variability is represented by quartile based coefficient of variation (QCV) calculated as (forth quartile—first quartile)/median. Each gene is represented by a blue dot. Black dots represent median QCV values of bins of 250 gene probes. Resistance mediating factors in NSCLC EGFR mutated cell lines are represented by red dots while their corresponding receptors are represented by orange squares. Both receptors and factors are significantly enriched in the group of genes with QCV above median (P-value<0.01 by hypergeometric test).

FIG. 15 demonstrate the correlations (R) between secreted factors' effect on resistance to BRAF inhibition and the expression of their corresponding receptors. rScore values of resistance mediating factors were correlated to the expression values of the corresponding receptors, across 15 μmelanoma BRAF(V600E) cell lines. Expression values of the receptors were adopted from the CCLE database (portals(dot)broadinstitute(dot)org/ccle). R—Pearson correlation coefficient. For the majority of factors, their effect on resistance to BRAF inhibitor cannot be explained by the expression level of their corresponding receptors.

FIGS. 16A-B demonstrate that mouse FGF2 and TNF factors can mediate the resistance of human cell lines to BRAF inhibition in a similar way to the human orthologues. FIG. 16A shows that human FGF2 (hFGF2—25 ng/ml) and mouse FGF2 (mFGF2—25 ng/ml) factors both mediated resistance to vemurafenib in UACC62 μmelanoma BRAF (V600E) cell line treated with the BRAFi inhibitor, vemurarenib (2 μM) in vitro. P-value of the rScore abrogation following the addition of FGFRi was calculated by two-sided t-test. ** P<0.01. FIG. 16B shows that mouse TNFa (mTNF—50 ng/ml) exhibited a strong effect on resistance to vemurafenib in UACC62 melanoma BRAF (V600E) cell line treated with vemurarenib (2 μM) in-vitro, similar to the results obtained in the in-vitro screen for human TNFa. The TNFR inhibitor R7050 does not abrogate the mouse TNF mediated resistance in the range of concentrations (all concentrations tested are not toxic to UACC62).

FIG. 17 shows body weight of UACC62-bearing mice during in-vivo experiment (described in FIG. 6D) with different drug combinations.

FIG. 18 demonstrate the results of an EVOC experiment of a BRAF (V600E) melanoma patient for prioritizing the co-targeting of potential innate resistance mechanisms. Patient responded temporarily to BRAFi/MEKi and was non-responsive to immune checkpoint inhibitors. Shown are tumor slices treated with BRAFi/MEKi with or without the addition of inhibitors for different potential innate resistance mechanisms found in the in-vitro screens (shown in FIG. 1E). Drugs and concentrations are similar to ones described in FIG. 6A. The percentage of viable cancer cells was morphologically assessed on H&E stained sections by a pathologist as the ratio between viable cancer cell area and total cancer area (viable cancer cells plus necrotic cancer cells). Scale bar represents 50 μm.

FIGS. 19A-C demonstrate ex-vivo and in-vivo experiments in EGFR-mutated NSCLC models. FIG. 19A demonstrate viability of EVOC of HCC4006 xenografts (known to have high level of pMET, thereby making MET pathway a potential resistance mechanism to EGFRi) following treatment with METi, EGFRi or a combination thereof. Vehicle—DMSO, METi-crizotinib 8 μM, EGFRi—erlotinib 4 μM. Each black dot in a given treatment condition represents a different tumor. P-values were calculated by one-sided t-test. * P<0.05. Error bars represent standard error. FIG. 19B shows representative images from FIG. 19A. Viability percentage is presented per treatment. Scale bar represents 50 μm. FIG. 19C shows body weight of H1975 bearing mice during the in-vivo experiment described in FIG. 7A.

FIGS. 20A-B demonstrate the use of EVOC to test co-targeting of somatic driver mutations for overcoming mechanisms of innate resistance. FIG. 20A demonstrate viability of EVOC of ESO26 xenografts (known to harbor an activating mutation in PIK3CA (Q546H), thereby making PI3K pathway as a potential resistance mechanism to EGFR/HER2i) following treatment with PI3Ki, EFGR/HER2i or HER3i or a combination thereof. Vehicle—DMSO, EGFR/HER2i-Afatinib 4 μM, PI3Ki—GDC-0941 10 μM, HER3i—Pertuzumab 20 μg/ml. Each black dot in a given treatment condition represents a different tumor. P-values were calculated by a one-sided t-test. * P<0.05. Error bars represent standard error. FIG. 20B shows representative images from FIG. 20A. Viability percentage is given per treatment combination. Scale bar—50 μm.

FIGS. 21A-C demonstrate implementation of integrative precision therapy for improving treatment efficacy in human patients. Freshly resected biopsy was sliced, and cultured ex vivo. Following 4 days of drug treatment, slices were fixed and embedded in paraffin blocks. FFPE slices were stained by H&E, and the percentage of viable cancer cells was morphologically assessed on H&E stained sections by a pathologist as the ratio between viable cancer cell area and total cancer area (viable cancer cells plus necrotic cancer cells). The most representative region is presented per treatment combination. FIG. 21A demonstrates results of an EVOC of a colorectal BRAF (V600E) patient. Vehicle—DMSO, FGFRi—AZD4547 2 μM, HER2i—trastuzumab 30 g/ml, BRAFi/MEKi—Dabrafenib 4 μM/trametinib 0.5 μM. Scale bar represents 50 μm. FIG. 21B demonstrates the results of an EVOC of a treatment-naïve, NSCLC male patient with poorly differentiated adenocarcinoma. Vehicle—DMSO, EGFRi—osimertinib 4 μM, FGFRi-AZD4547 2 μM, crizotinib—4 μM. Scale bar represents 50 μm. FIG. 21C demonstrates the results of an EVOC of a core biopsy taken from a liver metastasis of a NSCLC female patient who became refractory to osimertinib (EGFR mutation: exon19 del). Vehicle—DMSO, EGFRi-osimertinib 4 μM, FGFRi—AZD4547 2 μM, carboplatin—25 μM. Scale bar represents 50 μm.

### DESCRIPTION OF SPECIFIC EMBODIMENTS OF THE INVENTION

The present invention, in some embodiments thereof, relates to relates to combined treatment for cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Precision anti-cancer therapy, where drugs are tailored to patient-specific genetic abnormalities, has improved response rates over the last decades. Nevertheless, frequently the immediate response to treatment is suboptimal because of multiple mechanisms of innate resistance to the anti-cancer therapy administered.

Integrating precision therapy with targeting of tumor-specific mechanisms of innate resistance may maximize the response to treatment. Yet, challenges associated with determining tumor-specific mechanisms of resistance have hampered the use of such an integrative therapy in the clinic.

Whilst reducing specific embodiments of the present invention to practice, the present inventors were interested in demonstrating that personalized anti-cancer treatment based on both tumor-specific anti-cancer treatment and tumor specific innate resistance/sensitivity mechanisms to the anti-cancer drug may improve response to treatment. Following, the present inventors found out that ex vivo organ culture (EVOC) can be used to implement such an integrative therapy because it preserves the complex tumor composition, making it possible to functionally select drugs for overcoming mechanisms of resistance or for increasing sensitivity.

As is shown in the Examples section which follows, using a secretome screen the present inventors characterized the landscape of innate resistance/sensitivity mechanisms to several targeted anti-cancer therapies in multiple human cell lines of several cancer types (Example 1). However, the results also demonstrated that prioritization of the relevant patient-specific innate resistance mechanisms is challenging due to multiple variables (Example 2). To address these obstacles, the present inventors proposed EVOC as a functional approach to test combinations of an anti-cancer drug with agents that co-target the potential innate resistance/sensitivity mechanisms to the anti-cancer drug (Example 3). Indeed, EVOCs from several mice cancer xenograft models as well as from human fresh biopsies were able to prioritize such drug combinations and provide, in a clinically relevant time scale, an efficient prediction for treatment effectiveness, leading to better response to the anti-cancer therapies in the mice xenograft models.

Thus, for example, when considering administration of a specific anti-cancer drug for any given patient and tumor, specific embodiments of the invention suggest the use of the EVOC system to tailor a combined treatment by co-targeting tumor- and patient-specific potential mechanisms of resistance/sensitivity to the anti-cancer drug of choice.

Thus, according to an aspect of the present invention there is provided a method of selecting or determining therapeutic efficacy of a combination of agents for the treatment of cancer in a subject in need thereof, the method comprising:
(i) culturing a cancerous tissue of the subject in an ex-vivo organ culture (EVOC) in the presence of a combination of an anti-cancer agent and an additional agent, said additional agent is inhibiting expression and/or activity of a target conferring innate resistance to said anti-cancer agent or increasing expression and/or activity of a target conferring innate sensitivity to said anti-cancer agent; and
(ii) determining an anti-cancer effect of said combination on said tissue, wherein responsiveness of said tissue to said combination indicates said combination is efficacious for the treatment of said cancer in said subject.

As used herein the phrase “subject” refers to a mammalian subject (e.g., human being) who is diagnosed with the disease (i.e. cancer). Veterinary uses are also contemplated. The subject may be of any gender and any age including neonatal, infant, juvenile, adolescent, adult and elderly adult.

The terms “cancer” and “cancerous” describe the physiological condition in mammals that is typically characterized by unregulated cell growth. As used herein, the terms “cancer” and “cancerous” refers to any solid tumor, cancer metastasis and/or a solid pre-cancer.

Examples of cancer include but are not limited to, carcinoma, blastoma, sarcoma and lymphoma. More particular examples of such cancers include squamous cell cancer, lung cancer (including small-cell lung cancer, non-small-cell lung cancer, adenocarcinoma of the lung, and squamous carcinoma of the lung), glioma, melanoma cancer, cancer of the peritoneum, hepatocellular cancer, gastric, gastro esophageal or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, soft tissue sarcoma, kidney or renal cancer, prostate cancer, vulval cancer, thyroid cancer, hepatic carcinoma, Kaposi's sarcoma carcinoid carcinoma, and various types of head and neck cancer.

Precancers are well characterized and known in the art (refer, for example, to Berman J J. and Henson DE., 2003. Classifying the precancers: a metadata approach. BMC Med Inform Decis Mak. 3:8). Examples of precancers include but are not limited to include acquired small precancers, acquired large lesions with nuclear atypia, precursor lesions occurring with inherited hyperplastic syndromes that progress to cancer, and acquired diffuse hyperplasias and diffuse metaplasias. Non-limiting examples of small precancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia).

Non-limiting examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Non-limiting examples of precursor lesions occurring with inherited hyperplastic syndromes that progress to cancer include atypical mole syndrome, C cell adenomatosis and MEA. Non-limiting examples of acquired diffuse hyperplasias and diffuse metaplasias include Paget's disease of bone and ulcerative colitis.

According to specific embodiments, the cancer is selected from the group consisting of melanoma, non-small cell lung cancer, ovarian cancer, breast cancer, pancreatic cancer, esophageal cancer, colorectal cancer and prostate cancer.

According to specific embodiments, the cancer is selected from the group consisting of melanoma, colorectal cancer, non-small cell lung cancer and esophageal cancer.

According to specific embodiments, cells of the cancer comprise a mutation associated with responsiveness to the anti-cancer agent of choice. Such mutations are known to the skilled in the art and depend on the anti-cancer agent. For example, BRAF (V600E)-mutated cancers such as melanoma or colorectal cancer are known to respond to BRAF/MEK inhibitors (e.g. dabrafenib, vemurafenib, trametinib, PLX4720 PD184352); EGFR (i.e L858R, exon19 deletions, T790M) mutated cancers such as NSCLC are known to respond to EGFR inhibitors (e.g. afatinib, osimertinib, gefitinib, erlotinib); PIK3CA (i.e Q546H) mutated or PTEN loss cancers such as esophageal or ovarian cancers are known to respond to PI3K inhibitors (e.g pictilisib, ZSTK474), HER2 amplified cancers such as breast or esophageal cancers are known to respond to HER2/HER3 inhibitors (e.g lapatinib, trastuzumab, pertuzumab).

Thus, according to specific embodiments, the method is effected in combination with genetic profiling. Non-limiting examples of suitable profiling technology include DNA sequencing, RNA sequencing and microarray techniques.

According to specific embodiments, the cancer is selected from the group consisting of ovarian cancer, esophageal cancer, PDAC, BRAF wild-type melanoma, prostate cancer, breast cancer, BRAF mutated colorectal cancer, BRAF mutated melanoma and EGFR mutated NSCLC.

According to specific embodiments, the cancer is selected from the group consisting of BRAF mutated melanoma, EGFR mutated NSCLC, PDAC and BRAF wild-type melanoma.

According to specific embodiments, the cancer is selected from the group consisting of cancer is selected from the group consisting of BRAF mutated melanoma, EGFR mutated NSCLC, PDAC, ovarian cancer, esophageal cancer, prostate cancer, breast cancer, BRAF mutated colorectal cancer and BRAF wild-type melanoma.

As used herein the term “tissue” refers to part of a solid organ (i.e., not blood) of an organism having some vascularization that includes more than one cell type and maintains at least some macro structure of the in-vivo tissue from which it was excised.

Examples include, but are not limited to, ovarian tissue, colorectal tissue, lung tissue, pancreatic tissue, breast tissue, brain tissue, retina, skin tissue, bone, cardiac tissue and renal tissue. According to specific embodiments, the tissue is selected from the group consisting of ovarian, colorectal, lung, pancreas, gastric, gastro esophageal and breast. According to specific embodiments, the tissue is selected from the group consisting of ovarian, colorectal, lung, pancreas gastric, gastro esophageal, breast, liver, cartilage and bone. According to specific embodiments the tissue is a metastatic cancer tissue obtained from sites such as, but not limited to the liver, the bone, the lung and the peritoneum.

According to specific embodiments the tissue is obtained surgically or by biopsy, laparoscopy, endoscopy or as xenograft or any combinations thereof.

The tissue or the tissue slice to some embodiments of the present invention can be freshly isolated or stored e.g., at 4° C. or cryopreserved (i.e. frozen) at e.g. liquid nitrogen.

According to specific embodiments, the tissue or the tissue slice is freshly isolated (i.e., not more than 24 hours after retrieval from the subject and not subjected to preservation processes).

The tissue may be cut and cultured directly following tissue extraction (i.e. primary tissue) or following implantation in an animal model [i.e. a patient-derived xenograft (PDX)], each possibility represents a separate embodiment of the present invention.

Thus, according to specific embodiments, the method further comprises obtaining the tissue from the subject or from the animal model comprising the tissue.

As used herein the phrase “patient-derived xenograft (PDX)” refers to tissue generated by the implantation of a primary tissue into an animal from a different species relative to the donor of the primary tissue. According to specific embodiments the PDX is a tissue generated by implantation of a human primary tissue (e.g. cancerous tissue) into an immunodeficient mouse.

As used herein the term “ex-vivo organ culture (EVOC) system”, also known as “ex-vivo organotypic slice culture system” or “ex-vivo tissue slice culture system” refers to cultures of precision-cut slices of the patient's tumor used in cancer biology. EVOC has been used for diverse applications including the study of drug toxicity, viral uptake, susceptibility of tumors to radiation or specific anti-cancer drugs [see e.g. Vaira et al. (2010) Proc. Natl. Acad. Sci. U.S.A 107, 8352-8356; Vickers et al. (2004) Chem. Biol. Interact. 150, 87-96; de Kanter et al. (2002) Curr. Drug Metab. 3, 39-59; Stoff-Khalili et al. (2005) Breast Cancer Res. BCR 7, R1141-1152; Merz et al. (2013) Neuro-Oncol. 15, 670-681; Gerlach et al. (2014)Br. J. Cancer 110, 479-488; Meijer et al. (2013) Br. J. Cancer 109, 2685-2695; Grosso et al. (2013) Cell Tissue Res. 352, 671-684; Vaira et al. (2010) PNAS 107, 8352-8356; Roife et al. (2016) Clin. Cancer Res. June 3, 1-10; Maund et al. (2014) Lab. Invest. 94, 208-221; Vickers et al. (2004) Toxicol Sci. 82(2):534-44; Zimmermann et al. (2009) Cytotechnology 61(3): 145-152); Parajuli et al. (2009) In Vitro Cell.Dev.Biol. —Animal 45:442-450; Koch et al. (2014) Cell Communication and Signaling 12:73; Graaf et al. Nature Protocols (2010) 5: 1540-1551; Majumder et al. Nat. Commun. 6, 6169 (2015); US Patent Application Publication Nos: US2014/0228246, US2010/0203575 and US2014/0302491; and International Patent Application Publication No: WO2002/044344 and WO2018/185760, the contents of which are incorporated herein by reference in their entirety. A non-limiting example of an EVOC system that can be used with specific embodiments of the invention is described in details in the Examples section which follows, which serves as an integral part of the specification.

According to specific embodiments, the EVOC system is the one described in International Patent Application Publication No: WO2018/185760.

As used herein, the phrase “precision-cut tissue slice” refers to a viable slice obtained from an isolated solid tissue with reproducible, well defined thickness (e.g. ±5% variation in thickness between slices).

Typically, the tissue slice is a mini-model of the tissue which contains the cells of the tissue in their natural environment and retains the three-dimensional connectivity such as intercellular and cell-matrix interactions of the intact tissue with no selection of a particular cell type among the different cell type that constitutes the tissue or the organ.

The slice section can be cut in different orientations (e.g. anterior-posterior, dorsal-ventral, or nasal-temporal) and thickness. The size/thickness of the tissue section is based on the tissue source and the method used for sectioning. According to specific embodiment the thickness of the precision-cut slice allows maintaining tissue structure in culture.

According to specific embodiments the thickness of the precision-cut slice allows full access of the inner cell layers to oxygen and nutrients, such that the inner cell layers are exposed to sufficient oxygen and nutrients concentrations.

According to specific embodiments the thickness of the precision-cut slice allows full access of the inner cell layers to oxygen and nutrients, such that the inner cell layers are exposed to the same oxygen and nutrients concentrations as the outer cell layers.

According to specific embodiments, the precision-cut slice is between 50-1200 μm, between 100-1000 μm, between 100-500 μm, between 100-300 μm, or between 200-300 μm.

Methods of obtaining tissue slices are known in the art and described for examples in the Examples section which follows and in e.g. International Patent Application Publication No: WO2018/185760; Roife et al. (2016) Clin. Cancer Res. June 3, 1-10; Vickers et al. (2004) Toxicol Sci. 82(2):534-44; Zimmermann et al. (2009) Cytotechnology 61(3): 145-152); Koch et al. (2014) Cell Communication and Signaling 12:73; and Graaf et al. Nature Protocols (2010) 5: 1540-1551, the contents of each of which are fully incorporated herein by reference. Such methods include, but are not limited to slicing using a vibratome, agarose embedding followed by sectioning by a microtome, or slicing using a matrix.

According to specific embodiments, the culturing in the EVOC system maintains structure and viability of the precision-cut tissue slice for at least 2-10, 2-7, 2-5, 4-7, 5-7 or 4-5 days in culture. According to specific embodiments, at least 60%, at least 70%, at least 80% of the cells in the precision-cut tissue maintain viability following 4-5 days in culture as determined by e.g. morphology analysis of an optimal area of viability.

As used herein, the phrase “optimal area of viability” refers to a microscopic field of the tissue (e.g. in 20× magnification) in which the highest number of live cells per unit area are present, as assessed by a pathologist, in comparison to the immediate pre-EVOC sample of the same species.

Thus, according to specific embodiments, the culturing is effected for 2-10, 2-7, 2-5, 4-7, 5-7 or 4-5 days.

According to a specific embodiment, the culturing is effected for about 4 days.

The culture may be in a glass, plastic or metal vessel that can provide an aseptic environment for tissue culturing. According to specific embodiments, the culture vessel includes dishes, plates, flasks, bottles and vials. Culture vessels such as COSTAR®, NUNC® and FALCON® are commercially available from various manufacturers.

The culture medium used by the present invention can be a water-based medium which includes a combination of substances such as salts, nutrients, minerals, vitamins, amino acids, nucleic acids and/or proteins such as cytokines, growth factors and hormones, all of which are needed for cell proliferation and are capable of maintaining structure and viability of the tissue. For example, a culture medium can be a synthetic tissue culture medium such as DMEM/F12 (can be obtained from e.g. Biological Industries), M199 (can be obtained from e.g. Biological Industries), RPMI (can be obtained from e.g. Gibco-Invitrogen Corporation products), M199 (can be obtained from e.g. Sigma-Aldrich), Ko-DMEM (can be obtained from e.g. Gibco-Invitrogen Corporation products), supplemented with the necessary additives as is further described hereinunder. Preferably, all ingredients included in the culture medium of the present invention are substantially pure, with a tissue culture grade.

The skilled artisan would know to select the culture medium for each type of tissue contemplated.

According to specific embodiments, the tissue slice is placed on a tissue culture insert.

As used herein, the phrase “tissue culture insert” refers to a porous membrane suspended in a vessel for tissue culture and is compatible with subsequent ex-vivo culturing of a tissue slice. The pore size is capable of supporting the tissue slice while it is permeable to the culture medium enabling the passage of nutrients and metabolic waste to and from the slice, respectively. According to specific embodiments, the tissue slice is placed on the tissue culture insert, thereby allowing access of the culture medium to both the apical and basal surfaces of the tissue slice.

The cell culture insert may be synthetic or natural, it can be inorganic or polymeric e.g. titanium, alumina, Polytetrafluoroethylene (PTFE), Teflon, stainless steel, polycarbonate, nitrocellulose and cellulose esters. According to specific embodiments, the cell culture insert is a titanium insert. Cell culture inserts that can be used with specific embodiments of the invention are commercially available from e.g. Alabama R&D, Millipore Corporation, Costar, Corning Incorporated, Nunc, Vitron Inc. and SEFAR and include, but not limited to MA0036 Well plate Inserts, BIOCOAT™, Transwell®, Millicell®, Falcon®-Cyclopore, Nunc® Anapore, titanium-screen and Teflon-screen.

According to specific embodiments, the culturing is effected at a physiological temperature, e.g. 37° C., in a highly oxygenated humidified atmosphere containing at least 50%, at least 60%, at least 70%, at least 80% oxygen and e.g. 5% CO₂.

According to other specific embodiments, the highly oxygenated atmosphere contains less than 95% oxygen.

According to a specific embodiment, during the culturing process, the culture is agitated in a rotation facilitating intermittent submersion of the tissue slice in the culture medium.

The methods of some embodiments of the invention comprise culturing the cancerous tissue in the presence of a combination of an anti-cancer agent and an additional agent, as further described herein.

As used herein, the term “anti-cancer agent” refers to an agent capable of decreasing cancer growth and/or survival, for example by inducing cellular changes in a cancer cell or tissue (such as changes in cell viability, proliferation rate, differentiation, cell death, necrosis, apoptosis, senescence, transcription and/or translation rate of specific genes and/or changes in protein states e.g. phosphorylation, dephosphorylation, translocation and any combinations thereof), reducing the number of metastases, reducing blood supply to the tumor, promoting an immune response against the cancer cells or tissue.

Such anti-cancer agents are well known in the art and include, but not limited to, chemotherapeutic agents, radiotherapy agents, nutritional agents, immunotherapy agents and immune modulators; and may be, for example, small molecules, antibodies, peptides, toxins.

According to specific embodiments, the anti-cancer agent is a target therapy agent.

According to specific embodiments, the anti-cancer agent is a cytotoxic agent.

Non-limiting examples of anti-cancer drugs that can be used with specific embodiments of the invention are provided hereinbelow and in Example 1 of the Examples section which follows.

Non-limiting examples of anti-cancer drugs that can be used with specific embodiments of the invention include Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Non-limiting examples for anti-cancer approved drugs include: abarelix, aldesleukin, aldesleukin, alemtuzumab, alitretinoin, allopurinol, altretamine, amifostine, anastrozole, arsenic trioxide, asparaginase, azacitidine, AZD9291, AZD4547, AZD2281, bevacuzimab, bexarotene, bleomycin, bortezomib, busulfan, calusterone, capecitabine, carboplatin, carmustine, celecoxib, cetuximab, cisplatin, cladribine, clofarabine, cyclophosphamide, cytarabine, dabrafenib, dacarbazine, dactinomycin, actinomycin D, Darbepoetin alfa, Darbepoetin alfa, daunorubicin liposomal, daunorubicin, decitabine, Denileukin diftitox, dexrazoxane, dexrazoxane, docetaxel, doxorubicin, dromostanolone propionate, Elliott's B Solution, epirubicin, Epoetin alfa, erlotinib, estramustine, etoposide, exemestane, Filgrastim, floxuridine, fludarabine, fluorouracil 5-FU, fulvestrant, gefitinib, gemcitabine, gemtuzumab ozogamicin, goserelin acetate, histrelin acetate, hydroxyurea, Ibritumomab Tiuxetan, idarubicin, ifosfamide, imatinib mesylate, interferon alfa 2a, Interferon alfa-2b, irinotecan, lenalidomide, letrozole, leucovorin, Leuprolide Acetate, levamisole, lomustine, CCNU, meclorethamine, nitrogen mustard, megestrol acetate, melphalan, L-PAM, mercaptopurine 6-MP, mesna, methotrexate, mitomycin C, mitotane, mitoxantrone, nandrolone phenpropionate, nelarabine, Nofetumomab, Oprelvekin, Oprelvekin, oxaliplatin, paclitaxel, palbociclib palifermin, pamidronate, pegademase, pegaspargase, Pegfilgrastim, pemetrexed disodium, pentostatin, pipobroman, plicamycin mithramycin, porfimer sodium, procarbazine, quinacrine, Rasburicase, Rituximab, sargramostim, sorafenib, streptozocin, sunitinib maleate, tamoxifen, temozolomide, teniposide VM-26, testolactone, thioguanine 6-TG, thiotepa, thiotepa, topotecan, toremifene, Tositumomab, Trametinib, Trastuzumab, tretinoin ATRA, Uracil Mustard, valrubicin, vinblastine, vinorelbine, zoledronate and zoledronic acid.

According to specific embodiments, the anti-cancer agent is selected from the group consisting of Gefitinib, Lapatinib, Afatinib, BGJ398, CH5183284, Linsitinib, PHA665752, Crizotinib, Sunitinib, Pazopanib, Imatinib, Ruxolitinib, Dasatinib, BEZ235, Pictilisib, Everolimus, MK-2206, Trametinib/AZD6244, Vemurafinib/Dabrafenib, CCT196969/CCT241161, Barasertib, VX-680, Nutlin3, Palbociclib, BI 2536, Bardoxolone, Vorinostat, Navitoclax (ABT263), Bortezomib, Vismodegib, Olaparib (AZD2281), Simvastatin, 5-Fluorouricil, Irinotecan, Epirubicin, Cisplatin and Oxaliplatin.

According to specific embodiments, the anti-cancer agent is selected from the group consisting of BRAF/MEK inhibitor inhibitors (e.g. dabrafenib, vemurafenib, trametinib, PLX4720 PD184352), EGFR inhibitor (e.g. afatinib, osimertinib, gefitinib, erlotinib), HmG-CoA reductase inhibitor (e.g. Simvastatin), Mdm2 inhibitor (e.g. Nutlin3) and Hsp90 inhibitor (e.g. 17AAG).

According to specific embodiments, the anti-cancer agent is selected from the group consisting of Mitosis inhibitor, DNA synthesis inhibitor, PI3K alpha inhibitor, BRAF/MEK inhibitor and EGFR inhibitor.

According to specific embodiments, the “additional agent” which is combined with the anti-cancer agent refers to an agent not known to have an anti-cancer effect per se as a single agent on the cancer to be treated as determined e.g. in an EVOC system; however it inhibits expression and/or activity of a target conferring innate resistance to the anti-cancer agent of choice or increases expression and/or activity of a target conferring innate sensitivity to the anti-cancer agent of choice.

The target of some embodiments of the invention may be identified by available databases, published literature, genetic profiling, screening assays and the like.

According to specific embodiments, the target has been identified in an in-vitro screening assay (e.g. using a cell line).

According to specific embodiments, the target is a secreted factor or protein.

According to specific embodiments, cells of the cancer express a receptor of the target.

According to specific embodiments, the additional agent inhibits expression and/or activity of a target conferring innate resistance to the anti-cancer agent.

As used herein, the term “innate resistance”, also known as “immediate resistance”, “upfront resistance”, “intrinsic resistance” or “primary resistance”, refers to resistance to a specific anti-cancer drug that exists in the patient prior to administration of the drug.

As used herein, the phrase “target conferring innate resistance to the anti-cancer agent” refers to a cellular pathway or a component thereof, which confers the innate resistance. Typically, the pathway is characterized by genetic mutations associated with the cancer. Alternatively, or additionally the target is a factor or a protein secreted by the tumor microenvironment and the like.

Table 3 hereinbelow provides non-limiting examples of targets that can be inhibited according to specific embodiments of the invention.

TABLE 3

| | | | |
|---|---|---|---|
| EGF | INS | FGF10 | TGFB3 |
| BTC | HGF | NTF4 | BMP10 |
| TGFA | FGF2 | LTA | OSM |
| HBEGF | FGF9 | TNF | CNTF |
| EPGN | EMAPII | TNFRSF1B | PRL |
| Soluble EGFR | FGF4 | TNFSF14 | ADM |
| MMP7 | FGF6 | IL1A | CCL1 |
| NRG1a | FGF18 | TGFB1 | EDN1 |
| NRG1b | FGF7 | TGFB2 | FOLR2 |

According to specific embodiments, the target conferring innate resistance to said anti-cancer agent is selected from the group consisting of, epigen (EPGN), soluble epidermal growth factor receptor (EGFR), endothelial-monocyte activating polypeptide II (EMAPII), matrix metallopeptidase 7 (MMP7), neurotrophin4 (NTF4), lymphotoxin alpha (LTA), TNF superfamily member 14 (TNFSF14), bone morphogenetic protein 10 (BMP10), ciliary neurotrophic factor (CNTF), C—C motif chemokine ligand 1 (CCL1) and folate receptor beta (FOLR2).

Tables 4A-B hereinbelow provide non-limiting examples of combinations of cancer type, a first anti-cancer agent and a target that can be inhibited according to specific embodiments of the invention.

TABLE 4A

| | | | Agent inhibiting a target | |
|---|---|---|---|---|
| | | | conferring innate resistance to | |
| | First anti-cancer agent | | the first anti-cancer agent | |
| | | exemplary | | target |
| Cancer Type | target | primary drug | target | antagonist |
| Melanoma BRAF | BRAF | Dabrafenib | TGFA | Gefitinib |
| (V600E) | | PLX4720 | HBEGF | Erlotinib |
| | | Vemurafenib | EGFR | Afatinib |
| | MEK | PD184352 | MMP7 | Neratinib |
| | | Trametinib | | WZ4002 |
| | BRAF/MEK | Dabrafebin + | NRG1b | Lapatinib |
| | | Trametinib | | trastuzumab |
| | | PLX4720 + | | pertuzumab |
| | | PD184352 | INS | Linsitinib |
| | | | HGF | Crizotinib |
| | | | FGF2 | AZD4547 |
| | | | FGF9 | PD173074 |
| | | | EMAPII | |
| | | | FGF4 | |
| | | | FGF6 | |
| | | | FGF18 | |
| | | | FGF7 | |
| | | | NTF4 | ANA-12 |
| | | | | (anti TrkB) |
| | | | LTA | R7050 |
| | | | TNF | CAPE |
| | | | TNFRSF1B | |
| | | | TNFSF14 | |
| | | | IL1A | IRAK4-IN-2 |
| | | | | (anti IRAK4) |
| | | | TGFB1 | LY2109761 |
| | | | TGFB2 | |
| | | | TGFB3 | |
| | | | BMP10 | LDN-212854 |
| | | | | (anti BMPR, |
| | | | | ALK1) |
| | | | OSM | SC144 (anti |
| | | | CNTF | gp130) |
| | | | ADM | Rimegepant |
| | | | | (BMS-927711) |
| | | | | (anti CALCR) |
| | | | CCL1 | R243 (anti CCR8) |
| | | | EDN1 | Zibotentan |
| | | | | (ZD4054) (anti |
| | | | | endothelin) |
| | | | FOLR2 | Methotrexate |
| | | | | (anti DHFR) |
| NSCLC | EGFR | Gefitinib | NRG1b | Lapatinib |
| (EGFR | | Erlotinib | | trastuzumab |
| mutated) | | Afatinib | | pertuzumab |
| PDAC | | Neratinib | INS | Linsitinib |
| | | WZ4002 | HGF | Crizotinib |
| | | osimertinib | FGF2 | AZD4547 |
| | | | EMAPII | PD173074 |
| | | | FGF4 | |
| | | | PRL | LFA102 |
| | | | CCL1 | R243 (anti CCR8) |
| Ovarian Cancer | PI3Kalpha/delta | GDC0941 | EGF | Gefitinib |
| Esophageal | | ZSTK474 | BTC | Erlotinib |
| cancer | PI3Kalpha | BYL719 | TGFA | Afatinib |
| PDAC | | | HBEGF | Neratinib |
| Melanoma(BRAF | | | EPGN | WZ4002 |
| wt) | | | NRG1a | Lapatinib |
| Prostate cancer | | | NRG1b | trastuzumab |
| Breast cancer | | | | pertuzumab |
| | | | INS | Linsitinib |
| | | | FGF2 | AZD4547 |
| | | | | PD173074 |
| | | | LTA | R7050 |
| | | | | CAPE |
| | | | IL1A | IRAK4-IN-2 (anti |
| | | | | IRAK4) |
| | | | OSM | SC144 |
| Breast | EGFR/ERBB2 | Lapatinib | EGF | Gefitinib |
| (HER2 amp.) | | | BTC | Erlotinib |
| Esophageal | | | TGFA | Afatinib |
| cancer | | | HBEGF | Neratinib |
| | | | | WZ4002 |
| | | | NRG1a | trastuzumab |
| | | | NRG1b | pertuzumab |
| | | | HGF | Crizotinib |
| | | | FGF2 | AZD4547 |
| | | | FGF7 | PD173074 |
| | | | FGF10 | |
| | general | radiation | EGF | Gefitinib |
| | | | | Erlotinib |
| | | | | Afatinib |
| | | | | Neratinib |
| | | | | WZ4002 |
| | | | NRG1a | trastuzumab |
| | | | NRG1b | pertuzumab |
| | | | INS | Linsitinib |
| | | | TGFB2 | LY2109761 |
| CRC BRAF | BRAF | PLX4720 | EGF | Gefitinib |
| (V600E) | MEK | AZD6244 | BTC | Erlotinib |
| | | | TGFA | Afatinib |
| | | | HBEGF | Neratinib |
| | | | | WZ4002 |
| | | | NRG1b | Lapatinib |
| | | | | trastuzumab |
| | | | | pertuzumab |
| | | | INS | Linsitinib |
| | | | HGF | Crizotinib |
| | | | FGF2 | AZD4547 |
| | | | FGF9 | PD173074 |
| | | | FGF4 | |
| | | | FGF18 | |
| | | | FGF7 | |
| | | | FGF10 | |
| | | | LTA | R7050 |
| | | | | CAPE |
| | | | PRL | LFA102 |
| CRC BRAF | Mitosis | Docetaxel | EGF | Gefitinib |
| (V600E) | | | TGFA | Erlotinib |
| Breast | | | HBEGF | Afatinib |
| (HER2 amp.) | | | EPGN | Neratinib |
| | | | | WZ4002 |
| | | | NRG1b | Lapatinib |
| | | | | trastuzumab |
| | | | | pertuzumab |
| | | | TGFB1 | LY2109761 |
| | | | TGFB2 | |
| | | | TGFB3 | |
| | DNA synthesis | Doxorubicin | BMP10 | LDN-212854 (anti |
| | | | | BMPR, ALK1) |
| Ovarian cancer | Mitosis | Paclitaxel | TGFA | Gefitinib |
| | | | EGFR | Erlotinib |
| | | | | Afatinib |
| | | | | Neratinib |
| | | | | WZ4002 |
| | | | NTF4 | ANA-12 (anti |
| | | | | TrkB) |
| | DNA synthesis | Carboplatin | BMP10 | LDN-212854 (anti |
| | | | | BMPR, ALK1) |
| | | | OSM | SC144 |
| | | | CNTF | |
| | Ribonucleotide | Gemcitabine | EPGN | Gefitinib |
| | reductase | | | Erlotinib |
| | | | | Afatinib |
| | | | | Neratinib |
| | | | | WZ4002 |
| | | | NRG1b | trastuzumab |
| | | | | pertuzumab |
| | | | FGF10 | AZD4547 |
| | | | | PD173074 |
| | | | TGFB3 | LY2109761 |

TABLE 4B

| | | | Agent inhibiting a target | |
|---|---|---|---|---|
| | | | conferring innate resistance | |
| | First anti-cancer agent | | to the first anti-cancer agent | |
| | | exemplary | | target |
| Cancer Type | target | primary drug | target | antagonist |
| Melanoma | BRAF | Dabrafenib | TGFA | Gefitinib |
| BRAF | | PLX4720 | HBEGF | Erlotinib |
| (V600E) | | Vemurafenib | | Afatinib |
| | MEK | PD184352 | | Neratinib |
| | | Trametinib | | WZ4002 |
| | BRAF/MEK | Dabrafebin + | INS | Linsitinib |
| | | Trametinib | FGF9 | AZD4547 |
| | | PLX4720 + | FGF4 | PD173074 |
| | | PD184352 | FGF6 | |
| | | | FGF18 | |
| | | | FGF7 | |
| | | | TNF | R7050 |
| | | | TNFRSF1B | CAPE |
| | | | IL1A | IRAK4-IN-2 |
| | | | | (anti IRAK4) |
| | | | TGFB1 | LY2109761 |
| | | | TGFB2 | |
| | | | TGFB3 | |
| | | | OSM | SC144 (anti |
| | | | | gp130) |
| | | | ADM | Rimegepant |
| | | | | (BMS-927711) |
| | | | | (anti CALCR) |
| | | | EDN1 | Zibotentan |
| | | | | (ZD4054) (anti |
| | | | | endothelin) |
| NSCLC (EGFR | EGFR | Gefitinib | INS | Linsitinib |
| mutated) | | Erlotinib | EMAPII | PD173074 |
| PDAC | | Afatinib | FGF4 | |
| | | Neratinib | PRL | LFA102 |
| | | WZ4002 | | |
| Ovarian Cancer | PI3Kalpha/delta | GDC0941 | EGF | Gefitinib |
| Esophageal | | ZSTK474 | BTC | Erlotinib |
| cancer | PI3Kalpha | BYL719 | TGFA | Afatinib |
| PDAC | | | HBEGF | Neratinib |
| Melanoma(BRAF | | | INS | Linsitinib |
| wt) | | | IL1A | IRAK4-IN-2 |
| Prostate cancer | | | | (anti IRAK4) |
| Breast cancer | | | OSM | SC144 |
| Breast | EGFR/ERBB2 | Lapatinib | EGF | Gefitinib |
| (HER2 amp.) | | | BTC | Erlotinib |
| Esophageal | | | TGFA | Afatinib |
| cancer | | | HBEGF | Neratinib |
| | | | | WZ4002 |
| | | | HGF | Crizotinib |
| | | | FGF7 | AZD4547 |
| | | | FGF10 | PD173074 |
| | general | radiation | EGF | Gefitinib |
| | | | | Erlotinib |
| | | | | Afatinib |
| | | | | Neratinib |
| | | | | WZ4002 |
| | | | INS | Linsitinib |
| | | | TGFB2 | LY2109761 |
| CRC BRAF | BRAF | PLX4720 | EGF | Gefitinib |
| (V600E) | MEK | AZD6244 | BTC | Erlotinib |
| | | | HBEGF | Afatinib |
| | | | | Neratinib |
| | | | | WZ4002 |
| | | | INS | Linsitinib |
| | | | HGF | Crizotinib |
| | | | FGF9 | AZD4547 |
| | | | FGF4 | PD173074 |
| | | | FGF18 | |
| | | | FGF7 | |
| | | | FGF10 | |
| | | | PRL | LFA102 |
| CRC BRAF | Mitosis | Docetaxel | EGF | Gefitinib |
| (V600E) | | | TGFA | Erlotinib |
| Breast | | | HBEGF | Afatinib |
| (HER2 amp.) | | | | Neratinib |
| | | | | WZ4002 |
| | | | TGFB1 | LY2109761 |
| | | | TGFB2 | |
| | | | TGFB3 | |
| Ovarian cancer | Mitosis | Paclitaxel | TGFA | Gefitinib |
| | | | EGFR | Erlotinib |
| | | | | Afatinib |
| | | | | Neratinib |
| | | | | WZ4002 |
| | DNA syntehsis | Carboplatin | OSM | SC144 |
| | Ribonucleotide | Gemcitabine | FGF10 | AZD4547 |
| | reductase | | | PD173074 |
| | | | TGFB3 | LY2109761 |

According to specific embodiments, the cancer is a BRAF mutated melanoma cancer, the anti-cancer agent is a BRAF/MEK inhibitor and the target conferring innate resistance to the anti-cancer agent is selected from the group consisting of TGFA, HBEGF, NRG1b, HGF, FGF2, FGF9, EMAPII, FGF4, FGF6, FGF18, FGF7, LTA, TNF, ILIA, TGFB1, TGFB2, TGFB3 and OSM.

According to specific embodiments, the cancer is a BRAF mutated melanoma cancer, the anti-cancer agent is a BRAF/MEK inhibitor and the additional agent is a MET inhibitor, EGFR inhibitor, HER2 inhibitor, TGFBR inhibitor, gp130 inhibitor, FGFR inhibitor and/or TNFR inhibitor.

According to specific embodiments, the cancer is an EGFR mutated NSCLC cancer, the anti-cancer agent is a EGFR inhibitor and the target conferring innate resistance to said anti-cancer agent is selected from the group consisting of NRG1b, INS, HGF, FGF2, EMAPII and FGF4.

According to specific embodiments, the cancer is an EGFR mutated NSCLC cancer, the anti-cancer agent is an EGFR inhibitor and the additional agent is a FGFR inhibitor, INSR inhibitor, FGFR inhibitor and/or MET inhibitor.

According to specific embodiments, the cancer is an EGFR and PIK3CA mutated esophageal cancer, the anti-cancer agent is a PI3K inhibitor and the target conferring innate resistance to the anti-cancer agent is selected from the group consisting of EGF, BTC, TGFA, HBEGF, EPGN, NRG 1a and NRG1b.

According to specific embodiments, the cancer is an EGFR and PIK3CA mutated esophageal cancer, the anti-cancer agent is a PI3K inhibitor and the additional agent is a EGFR inhibitor, HER2 inhibitor, and/or HER3 inhibitor.

As used herein, the terms “inhibiting”, “inhibit” and “inhibitor”, which are interchangeably used herein, refer to a decrease of at least 5% in expression and/or activity of the target in the presence of the agent in comparison to same in the absence of the agent, as determined by e.g. PCR, ELISA, Western blot analysis, activity assay (e.g. enzymatic, kinase, binding), cell cycle arrest (as determined by e.g. flow cytometry), increased cell death (as determined by e.g. TUNEL assay, Annexin V).

According to a specific embodiment, the decrease is in at least 10%, 20%, 30%, 40% or even higher say, 50%, 60%, 70%, 80%, 90%, 95% or 100%.

Decreasing expression and/or activity of the target can be effected at the genomic (e.g. homologous recombination and site specific endonucleases) and/or the transcript level using a variety of molecules which interfere with transcription and/or translation (e.g., RNA silencing agents) or on the protein level (e.g., small molecules, aptamers, inhibitory peptides, antagonists, enzymes that cleave the polypeptide, antibodies and the like).

According to specific embodiments, the inhibitor affect the expression of the target. Such inhibitors are well known in the art and typically include nucleic acid molecules that mediate their function through genome editing or RNA silencing.

According to specific embodiments, the inhibitor affect the activity of the target. Such an inhibitor is typically a small molecule chemical, an antibody or a peptide.

The inhibition may be either transient or permanent.

According to specific embodiments, the inhibitor also encompasses an upstream activator inhibitor, a downstream effector inhibitor or a receptor/ligand inhibitor.

According to a specific embodiments, the inhibitor inhibits a receptor/ligand of the target.

According to a specific embodiment, the inhibitor specifically inhibits the target and not an upstream activator, a downstream effector or a receptor/ligand of the target.

Non-limiting examples of such inhibitors that can be used with specific embodiments of the invention are provided in Tables 4A-B hereinabove and in the Examples section which follows.

According to specific embodiments, the additional agent increases expression and/or activity of a target conferring innate sensitivity to the anti-cancer agent.

As used herein, the term “innate sensitivity”, also known as “immediate sensitivity”, “upfront sensitivity”, “intrinsic sensitivity” or “primary sensitivity”, refers to sensitivity to a specific anti-cancer drug that exists in the patient prior to administration of the drug.

As used herein, the phrase “target conferring innate sensitivity to the anti-cancer agent” refers to a cellular pathway or a component thereof, which confers the innate sensitivity. Typically, the pathway is characterized by genetic mutations associated with the cancer. Alternatively, or additionally the target is a factor or a protein secreted by the tumor microenvironment and the like.

Table 5 hereinbelow provides non-limiting examples of targets their expression and/or activity can be increased according to specific embodiments of the invention.

TABLE 5

| | | | |
|---|---|---|---|
| TGFB1 | CSF2 | EGFR | PYY |
| TGFB2 | IFNA2 | TNF | ACHE |
| TGFB3 | IL10 | PROK2 | APCS |
| BMP2 | PDGFB | RLN3 | COL4A1 |
| BMP4 | EFNA5 | AVP | VTN |

According to specific embodiments, the target conferring innate sensitivity to the anti-cancer drug is selected from the group consisting of Transforming Growth Factor Beta 1-3 (TGFB1-3), Colony Stimulating Factor 2 (CSF2), Interleukin 10 (IL10), Platelet Derived Growth Factor Subunit B (PDGFB), Ephrin A5 (EFNA5), Soluble Epidermal Growth Factor Receptor (EGFR), Prokineticin 2 (PROK2), Relaxin 3 (RLN3), Peptide YY (PYY), acetylcholinesterase (ACHE), Amyloid P Component, Serum (APCS), Collagen Type IV Alpha 1 Chain (COL4A1) and Vitronectin (VTN).

Tables 6A-B hereinbelow provide non-limiting examples of combinations of cancer type, a first anti-cancer agent and a target its expression and/or activity can be increased according to specific embodiments of the invention.

TABLE 6A

| | | | Agent inhibiting a target | |
|---|---|---|---|---|
| | | | conferring innate sensitivity | |
| | | | to the first anti-cancer agent | |
| | First anti-cancer agent | | | target |
| Cancer Type | target | primary drug | target | agonist |
| Melanoma | BRAF | Dabrafenib | TGFB1 | SRI-011381 |
| BRAF | | PLX4720 | TGFB2 | |
| (V600E) | | Vemurafenib | TGFB3 | |
| | | | BMP2 | BMP2 |
| | | | CFS2 | CFS2 |
| | MEK | PD184352 | IFNA2 | Interferon |
| | | | | receptor |
| | | Trametinib | | inducer 1 |
| | | | IL10 | IL 10 |
| | BRAF/MEK | Dabrafebin + | PDGFB | PDGFB |
| | | Trametinib | EFNA5 | EFNA5 |
| | | PLX4720 + | PROK2 | PROK2 |
| | | PD184352 | RLN3 | RLN3 |
| | | | AVP | AVP |
| | | | PYY | PYY |
| | | | ACHE | ACHE |
| | | | APCS | APCS |
| | | | COL4A1 | COL4A1 |
| | | | VTN | VTN |
| NSCLC | EGFR | Gefitinib | TGFB3 | SRI-011381 |
| (EGFR | | Erlotinib | BMP4 | sb4 |
| mutated) | | | | |
| PDAC | | | | |
| Ovarian | Mitosis | Paclitaxel | TNF | Resiquimod |
| cancer | | | | |
| Melanoma | HMG-COA | Simvastatin | EGFR | EGFR |
| BRAF (wt) | reductase | | | |
| | MDM2 | Nutlin3 | APCS | APCS |
| | Hsp90 | 17AAG | | |

TABLE 6B

| | | | Agent activating a target | |
|---|---|---|---|---|
| | | | conferring innate sensitivity | |
| Cancer | First anti-cancer agent | | to the first anti-cancer agent | |
| Type | target | primary drug | target | target agonist |
| Melanoma | BRAF | Dabrafenib | BMP2 | BMP2 |
| BRAF | | PLX4720 | AVP | AVP |
| (V600E) | | Vemurafenib | | |
| | MEK | PD184352 | | |
| | | Trametinib | | |
| | BRAF/MEK | Dabrafebin + | | |
| | | Trametinib | | |
| | | PLX4720 + | | |
| | | PD184352 | | |
| NSCLC | EGFR | Gefitinib | BMP4 | sb4 |
| (EGFR | | Erlotinib | | |
| mutated) | | | | |
| PDAC | | | | |

According to specific embodiments, the cancer is a BRAF mutated melanoma cancer, the anti-cancer agent is a BRAF/MEK inhibitor and the target conferring innate sensitivity to the anti-cancer drug is selected from the group consisting of TGFB1, TGFB2, TGFB3, BMP2, CFS2,IL10, RLN3 and ACHE.

According to specific embodiments, the cancer is an EGFR mutated NSCLC cancer or PDAC cancer, the anti-cancer agent is a mitosis inhibitor and the target conferring innate sensitivity to the anti-cancer drug is TGFB3 and/or BMP4.

According to specific embodiments, the cancer is an ovarian cancer, the anti-cancer agent is an EGFR inhibitor and the target conferring innate sensitivity to the anti-cancer drug is TNFa.

According to specific embodiments, the cancer is a BRAF wild-type melanoma, the anti-cancer agent is an MDM2 inhibitor or an Hsp90 inhibitor and the target conferring innate sensitivity to the anti-cancer drug is APCS.

As used herein, the term “increasing” or “increase” refers to an increase of at least 5% in expression and/or activity in the presence of the agent in comparison to same in the absence of the agent, as determined by e.g. PCR, ELISA, Western blot analysis, activity assay (e.g. enzymatic, kinase, binding), cell cycle arrest (as determined by e.g. flow cytometry), increased cell death (as determined by e.g. TUNEL assay, Annexin V).

According to a specific embodiment, the increase is in at least 10%, 20%, 30%, 40% or even higher say, 50%, 60%, 70%, 80%, 90%, 95%, 100% or more.

Increasing expression and/or activity of the target can be effected at the genomic level (i.e., activation of transcription via promoters, enhancers, regulatory elements), at the transcript level (i.e., correct splicing, polyadenylation, activation of translation) or at the protein level (i.e., post-translational modifications, interaction with substrates and the like).

Such agents are well known in the art and include e.g. an exogenous polynucleotide sequence designed and constructed to express at least a functional portion of the target, a compound which is capable of increasing the transcription and/or translation of an endogenous DNA or mRNA encoding target, an exogenous polypeptide including at least a functional portion of the target, a substrate, an agonistic antibody.

The increase may be either transient or permanent.

According to specific embodiments, the increasing agent also encompasses an agent increasing expression and/or activity of an upstream activator, a downstream effector or a receptor/ligand of the target.

According to a specific embodiments, the agent increases expression and/or activity of a receptor/ligand of the target.

According to a specific embodiment, the agent specifically increases expression and/or activity the target and not an upstream activator, a downstream effector or a receptor/ligand of the target.

Non-limiting examples of such agents that can be used with specific embodiments of the invention are provided in Tables 6A-B hereinabove and in the Examples section which follows.

The agent or the combination of agents may be added to the culture at various time points. According to specific embodiments, the combination is added to the culture 2-96 hours, 2-48 hours, 2-36, 2-24, 12-48, 12-36 or 12-24 hours following the beginning of the culture.

The combination may be added concomitantly or in a sequential manner.

According to specific embodiments, the anti-cancer agent and the additional agent are added to the culture concomitantly.

Culturing in the presence of the combination of agents may be effected throughout the whole culturing period from first drug addition or can be limited in time. Alternatively, or additionally, the drug or the drug combination may be added to the culture multiple times e.g. when the culture medium is refreshed.

Selection of the incubation time with the combination of agents that will result in detectable effect on the tissue as further described hereinbelow, is well within the capabilities of those skilled in the art.

According to specific embodiments, culturing with the combination of agents is effected from 24-120 hours, 48-120 hours, or 48-96 hours.

Selection of drug concentrations that will result in detectable effect on the tissue as further described hereinbelow, is well within the capabilities of skilled in the art and may be determined e.g. by preliminary examination or known data.

According to specific embodiments, several concentrations are tested in the same assay.

The number of tested concentrations can be at least 1, at least 2, at least 3, at least 5, at least 6, 1-10, 2-10, 3-10, 5-10, 1-5, 2-5 and 3-5 different concentrations in the same assay.

The number of samples repeats for each of the tested concentration can be 2, 3, 4, 5 or 6 repeats.

According to specific embodiments, for an anti-cancer drug targeting a tumor driver mutation, the working concentration is the maximal concentration which does not lead to cell death in cancer tissue without the targeted mutation.

Following the culturing, the method of some embodiments of the invention comprises determining the anti-cancer effect of the combination of agents on the tissue to thereby determine efficacy of the combination.

According to specific embodiments, the determining step is effected following a pre-determined culturing time. The culturing time may vary and determination of the culturing time that will result in detectable effect is well within the capabilities of those skilled in the art.

According to specific embodiments, the determining is effected within 2-10, 2-7, 2-5, 3-10, 3-7, 3-5 or 4-5 days of culturing.

According to a specific embodiment, the determining is effected within 3-5 days of culturing.

As used herein, the term “anti-cancer effect” refers to cellular changes in the cancerous tissue reflecting a decrease in tumor growth and/or survival such as changes in cell viability, proliferation rate, differentiation, cell death, necrosis, apoptosis, senescence, transcription and/or translation rate of specific genes and/or changes in protein states e.g. phosphorylation, dephosphorylation, translocation and any combinations thereof.

As used herein, the term “responsiveness” refers to the ability of an agent or a combination of agents to induce an anti-cancer effect in an EVOC system, as compared to same in the absence of the agent or the combination of agents.

According to specific embodiments, responsiveness is reflected by decreased cell viability, decreased proliferation rate, increased cell death and/or aberrant morphology as compared to same in the absence of the drug.

According to other specific embodiments the change is by at least 5%, by at least a 10%, at least 20%, at least 30%, at least 40%, at least 50%, at least 60%, at least 70%, at least 80%, at least 90%, at least 95%, at least 99% or at least 100% as compared to same in the absence of the agent or the combination of agents.

According to specific embodiments, responsiveness is increased responsiveness as compared to individual treatment with the anti-cancer agent or the additional agent, as determined by the EVOC system.

Methods of determining anti-cancer effect and responsiveness are known in the art and include for example:
Viability evaluation using e.g. the MTT test which is based on the selective ability of living cells to reduce the yellow salt MTT (3-(4, 5-dimethylthiazolyl-2)-2, 5-diphenyltetrazolium bromide) (Sigma, Aldrich St Louis, MO, USA) to a purple-blue insoluble formazan precipitate; the WST assay or the ATP uptake assay;
Proliferation evaluation using e.g. the BrDu assay [Cell Proliferation ELISA BrdU colorimetric kit (Roche, Mannheim, Germany] or Ki67 staining; Cell death evaluation using e.g. the TUNEL assay [Roche, Mannheim, Germany] the Annexin V assay [ApoAlert® Annexin V Apoptosis Kit (Clontech Laboratories, Inc., CA, USA)], the LDH assay, the Activated Caspase 3 assay, the Activated Caspase 8 assay and the Nitric Oxide Synthase assay;
Senescence evaluation using e.g. the Senescence associated-β-galactosidase assay (Dimri GP, Lee X, et al. 1995. A biomarker that identifies senescent human cells in culture and in aging skin in vivo. Proc Natl Acad Sci USA 92:9363-9367) and telomerase shortening assay;
Cell metabolism evaluation using e.g. the glucose uptake assay;
Various RNA and protein detection methods (which detect level of expression and/or activity); and
Morphology evaluation using e.g. the Haemaotxylin & Eosin (H&E) staining;

According to specific embodiments, the determining is effected by morphology evaluation, viability evaluation, proliferation evaluation and/or cell death evaluation.

According to specific embodiments, the determining is effected by morphology evaluation.

Morphology evaluation using H&E staining can provide details on e.g. cell content, size and density, ratio of viable cells/dead cells, ratio of diseased (e.g. tumor) cells/healthy cells, immune cells infiltration, fibrosis, nuclear size and density and integrity, apoptotic bodies and mitotic figures. According to specific embodiments effect of the drug on the tissue is determined by morphology evaluation by e.g. a pathologist.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

According to specific embodiments, the determined efficacy of the combination indicates suitability of the combination for the treatment of cancer in the subject.

Thus, according to an aspect of the present invention, there is provided a method of treating cancer in a subject in need thereof, the method comprising:
(a) selecting treatment or determining therapeutic efficacy of a combination of agents according to the method disclosed herein; and
(b) administering to said subject a therapeutically effective amount of a combination demonstrating efficacy for the treatment of said cancer in said subject,
thereby treating the cancer in the subject. In addition, the present inventors identified novel combination of agents that can be used for cancer treatment.

Thus, according to an additional or an alternative aspect of the present invention, there is provided a method of treating cancer in a subject in need thereof, the method comprising administering to the subject a therapeutically effective amount of a combination of an anti-cancer agent and an additional agent inhibiting expression and/or activity of a target selected from the group consisting of epigen (EPGN), soluble epidermal growth factor receptor (EGFR), endothelial-monocyte activating polypeptide II (EMAPII), matrix metallopeptidase 7 (MMP7), neurotrophin4 (NTF4), lymphotoxin alpha (LTA), TNF superfamily member 14(TNFSF14), bone morphogenetic protein 10 (BMP10), ciliary neurotrophic factor (CNTF), C—C motif chemokine ligand 1 (CCL1) and folate receptor beta (FOLR2), wherein cancerous tissue obtained from said subject demonstrates responsiveness to said combination in an ex-vivo organ culture (EVOC), thereby treating the cancer in the subject.

According to an additional or an alternative aspect of the present invention, there is provided a method of treating cancer in a subject in need thereof, the method comprising administering to the subject a therapeutically effective amount of a combination of an anti-cancer agent and an additional agent inhibiting expression and/or activity of a target, wherein said anti-cancer agent, said target and said cancer are selected from the group of combinations listed in Table 4B, and wherein cancerous tissue obtained from said subject demonstrates responsiveness to said combination of agents in an ex-vivo organ culture (EVOC), thereby treating the cancer in the subject.

According to an additional or an alternative aspect of the present invention, there is provided a method of treating cancer in a subject in need thereof, the method comprising administering to the subject a therapeutically effective amount of a combination of an anti-cancer agent and an additional agent increasing expression and/or activity of a target selected from the group consisting of Transforming Growth Factor Beta 1-3 (TGFB1-3), Colony Stimulating Factor 2 (CSF2), Interleukin 10 (IL10), Platelet Derived Growth Factor Subunit B (PDGFB), Ephrin A5 (EFNA5), soluble epidermal growth factor receptor (EGFR), Prokineticin 2 (PROK2), Relaxin 3 (RLN3), Peptide YY (PYY), acetylcholinesterase (ACHE), Amyloid P Component, Serum (APCS), Collagen Type IV Alpha 1 Chain (COL4A1) and Vitronectin (VTN), wherein cancerous tissue obtained from said subject demonstrates responsiveness to said combination in an ex-vivo organ culture (EVOC), thereby treating the cancer in the subject.

According to an additional or an alternative aspect of the present invention, there is provided a method of treating cancer in a subject in need thereof, the method comprising administering to the subject a therapeutically effective amount of a combination of an anti-cancer agent and an additional agent increasing expression and/or activity of a target, wherein said anti-cancer agent, said target and said cancer are selected from the group of combinations listed in Table 6B, and wherein cancerous tissue obtained from said subject demonstrates responsiveness to said combination of agents in an ex-vivo organ culture (EVOC), thereby treating the cancer in the subject.

According to an additional or an alternative aspect of the present invention, there is provided a method of treating cancer in a subject in need thereof, the method comprising administering to the subject a therapeutically effective amount of a combination of agents selected from the group of combinations listed in Table 7, wherein cancerous tissue obtained from said subject demonstrates responsiveness to said combination of agents in an ex-vivo organ culture (EVOC), thereby treating the cancer in the subject.

According to an additional or an alternative aspect of the present invention, there is provided a method of treating cancer in a subject in need thereof, the method comprising administering to the subject a therapeutically effective amount of a combination of agents, wherein said combination of agents and said cancer are selected from the group of combinations listed in Table 8, and wherein cancerous tissue obtained from said subject demonstrates responsiveness to said combination of agents in an ex-vivo organ culture (EVOC), thereby treating the cancer in the subject.

Tables 7-8 hereinbelow provide non-limiting examples of combinations of agents that can be used with specific embodiments of the invention

TABLE 7
agent

| Primary anti-cancer agent | | Secondary agent | |
|---|---|---|---|
| Target | Example | Target | Example |
| BRAF/MEK | vemurafenib/ | TNFR inhibitor | R-7050 |
| inhibitor | trametinib | TNFRinhibitor + | R-7050 + AZD4547 |
| | | FGFR inhibitor | |
| | | gp130 inhibitor | SC144 |
| | | PRLR inhibitor | LFA102 |
| | | TGFBR agonist | SRI-011381 |
| | | BMPR1A agonist | bone morphogenetic |
| | | | protein 2 (BMP2) |
| | | CSF2R agonist | colony stimulating |
| | | | factor 2 (CFS2) |
| | | IL10R agonist | interleukin 10 |
| | | | (IL10) |
| | | PROKR agonist | prokineticin 2 |
| | | | (PROK2) |
| | | RXFP3 agonist | relaxin 3 (RLN3) |
| | | acetylcholine esterase | |
| | | (ACHE) | |
| | | amyloid P-component | |
| | | serum (APCS) | |
| | | peptide YY (PYY) | |
| | | Vitronectin (VTN) | |
| EGFR | Erlotinib | PRLR inhibitor | LFA102 |
| inhibitor | | TGFBR agonist | SRI-011381 |
| PI3K | GDC0941 | TNFR inhibitor | R7050 |
| inhibitor | | | |
| PI3Kalpha | BYL719 | | |
| mitosis | Docetaxel | TNFa agonist | Resiquimod |
| inhibitor | | | |
| Hsp90 | 17AAG | APCS | |
| HMG-CoA | Simvastatin | EGFR (solube) | |
| reductase | | | |

TABLE 8

| | Primary anti-cancer agent | | Secondary agent | |
|---|---|---|---|---|
| Cancer Type | Target | Example | Target | Example |
| PDAC | EGFR inhibitor | erlotinib | MET | Crizotinib |
| | | | inhibitor | |
| | | | FGFR | AZD4547 |
| | | | inhibitor | |
| Ovarian Cancer | PI3Kalpha/delta | BYL719/GDC0941 | gp130 | SC144 |
| Esophageal cancer | inhibitor | | | |
| PDAC | | | | |
| Melanoma(BRAF | | | inhibitor | |
| wt) | | | | |
| Prostate cancer | | | | |
| Breast cancer | | | | |
| CRC BRAF | BRAF/MEK | vemurafenib/ | FGFR | AZD4547 |
| (V600E) | inhibitor | trametinib | inhibitor | |
| | mitosis inhibitor | Docetaxel | TGFBR | LY2109761 |
| | | | inhibitor | |
| Ovarian cancer | DNA synthesis | Carboplatin | gp130 | SC144 |
| | inhibitor | | inhibitor | |

Determination of a therapeutically effective amount of the combination is well within the capability of those skilled in the art. The dosage may vary depending upon the drug chosen, the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

According to an additional or an alternative aspect of the invention, there is provided an article of manufacture comprising as active ingredients the combination of agents of some embodiments disclosed herein.

According to specific embodiments, the article of manufacture is identified for the treatment of cancer.

According to specific embodiments, the combination of agents are provided in a co-formulation.

According to other specific embodiments, each of the agents is provided in a separate formulation.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition (i.e. cancer), substantially ameliorating clinical or aesthetical symptoms of a condition or substantially preventing the appearance of clinical or aesthetical symptoms of a condition. Those of skill in the art will understand that various methodologies and assays can be used to assess the development of a condition (i.e. cancer), and similarly, various methodologies and assays may be used to assess the reduction, remission or regression of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Cell lines—All cell lines (Table 1 hereinbelow) were cultured in DMEM supplemented with 100 units/ml Penicillin and Streptomycin, 2 μmM Glutamine, 1 μmM Pyruvate and 10% FCS, and maintained in a humidified incubator at 37° C. and 5% CO₂. Cells were routinely tested for Mycoplasma contamination by PCR. To generate stable cell lines constitutively expressing GFP, cells were infected with lentivirus bearing plasmid pLEX_TRC206 and sorted by FACS to enrich for GFP positive population. For the in-vitro experiments, human cancer cell lines which constitutively expressed GFP were treated with various cytotoxic and targeted drugs (see Table 1 hereinbelow); and the effect of different secreted factors (Table 2 hereinbelow) on the innate resistance to a drug based on the cells' GFP level was determined.

TABLE 1
List of cell lines and drugs

| group name | tissues | cell lines | target | drugs |
|---|---|---|---|---|
| Melanoma(BR | Melanoma | A101D(ATCC, CRL- | BRAF, MEK, | Dabrafenib(ADOOQ, |
| AFV600E)- | (BRAFV600E) | 7898), A2058(ATCC, | BRAF + MEK | A11281), |
| BRAF, MEK | | CRL-11147), | | PLX4720(Chemietek |
| | | A375(ATCC, CRL-1619), | | CT-P4720), |
| | | C32(ATCC, CRL-1585), | | Vemurafenib(LC |
| | | COLO-679(ECACC, | | Laboratories, V-2800), |
| | | 87061210), COLO- | | PD184352(Santa cruz, |
| | | 800(ECACC, 93051123), | | sc-202759A), |
| | | COLO-829(ATCC, CRL- | | Trametinib(LC |
| | | 1974), G361(ATCC, | | Laboratories, T-8123), |
| | | CRL-1424), | | Dabrafenib(ADOOQ, |
| | | K029AX(Expasy | | A11281) + |
| | | CVCL_8784), Malme- | | Trametinib(LC |
| | | | | Laboratories, |
| | | 3M(ATCC, HTB-64), | | T-8123), |
| | | SK-MEL-19(Expasy, | | PLX4720(Chemietek, |
| | | CVCL_6025), SK-MEL- | | CT-P4720) + |
| | | 28(ATCC, HTB-72), SK- | | PD184352(Santa |
| | | MEL-5(ATCC, HTB-70), | | cruz, sc-202759A) |
| | | UACC62(Expasy, | | |
| | | CVCL_1780), WM3163, | | |
| | | WM3218, WM3228, | | |
| | | WM3259, | | |
| | | WM3482(Rockland, | | |
| | | WM3482-01-0001), | | |
| | | WM3627, WM88(Expasy, | | |
| | | CVCL_6805), | | |
| | | WM983B(Expasy, | | |
| | | CVCL_6809) | | |
| NSCLC/PDA | NSCLC, PDAC | H1975(ATCC, CRL- | EGFR, EGFR + | Gefitinib(LC |
| C-EGFR, HER2 | | 5908), H3255(ATCC, | HER2 | Laboratories, G-4408), |
| | | CRL-2882), | | Erlotinib(LC |
| | | HCC2935(ATCC, CRL- | | Laboratories, E-4007), |
| | | 2869), HCC827(ATCC, | | WZ4002(Selleck, |
| | | CRL-2868), PC9(Expasy, | | S1173), Afatinib(LC |
| | | CVCL_B260), | | Laboratories, A-8644), |
| | | PC9(Expasy, | | Neratinib(Selleck, |
| | | CVCL_B260) (GR4), | | S2150) |
| | | Panc1(ATCC, CRL- | | |
| | | 1469), AsPC1(ATCC, | | |
| | | CRL-1682), | | |
| | | BxPC3(ATCC, CRL- | | |
| | | 1687) | | |
| various tissue- | Ovarian, | A2780(Expasy, | PI3Kalpha/delta, | GDC-0941(cayman, |
| PI3K | Esophageal, PDAC, | CVCL_0134), | PI3K alpha | 11600-10), |
| | Melanoma(BRAF wt), | Fuov1(Expasy, | | ZSTK474(AdooQ, |
| | Prostate, Breast | CVCL_2047), | | A11014), BYL- |
| | | IGROV1 (Expasy, | | 719(ChemieTek, CT- |
| | | CVCL_1304), | | BYL719) |
| | | OE19(ECACC, | | |
| | | 96071721), NCI- | | |
| | | N87(ATCC, CRL-5822), | | |
| | | ESO26(Expasy, | | |
| | | CVCL_2035), | | |
| | | Panc1(ATCC, CRL- | | |
| | | 1469), AsPC1(ATCC, | | |
| | | CRL-1682), | | |
| | | BxPC3(ATCC, CRL- | | |
| | | 1687), 120T, 24T, | | |
| | | LNCaP(ATCC, CRL- | | |
| | | 1740 (Clone FGC)), | | |
| | | PC3(ATCC, CRL-1435), | | |
| | | MCF-7(ATCC, HTB-22), | | |
| | | T47D(ATCC, HTB-133), | | |
| | | MDA-MB-453(ATCC, | | |
| | | HTB-131), SK-BR- | | |
| | | 3(ATCC, HTB-30) | | |
| Melanoma | Melanoma(BRAF wt) | 108T, 120T, 24T, 32T, | MDM2, | Nutlin-3(Cayman, |
| BRAF(wt)- | | 76T, 96T, | CDK4/6, | 10004372), |
| various non- | | WM3211(Rockland, | HDAC, HMG- | Palbociclib(Sigma, |
| specific targets | | WM3211-01-0001) | CoA | PZ0199), |
| | | | reductase, | Vorinostat(Selleck, |
| | | | HSP90 | S1047), |
| | | | | Simvastatin(Bio Vision, |
| | | | | 1693-50), 17- |
| | | | | AAG(LC Laboratories, |
| | | | | A-6880) |
| Ovarian-DNA | Ovarian | Cov318(Expasy, | DNA syntehsis | Carboplatin(Sigma, |
| synthesis | | CVCL_2419), | | C2538) |
| | | Kuramochi(Expasy, | | |
| | | CVCL_1345), OvCAR- | | |
| | | 432(Expasy, | | |
| | | CVCL_3769), | | |
| | | OvSAHO(Expasy, | | |
| | | CVCL_3114) | | |
| Breast/Esopha | Breast(HER2), | SK-BR-3(ATCC, HTB- | EGFR + HER2 | Lapatinib(LC |
| geal-EGFR, HER2 | Esophageal | 30), HCC1954(ATCC, | | Laboratories, L-4804) |
| | | CRL-2338), MDA-MB- | | |
| | | 453(ATCC, HTB-131), | | |
| | | ESO26(Expasy, | | |
| | | CVCL_2035), NCI- | | |
| | | N87(ATCC, CRL-5822), | | |
| | | OE19(ECACC, | | |
| | | 96071721) | | |
| Ovarian- | Ovarian | Cov318(Expasy, | Ribonucleotide | Gemcitabine(Sigma, |
| Ribonucleotide | | CVCL_2419), | reductase | G6423) |
| reductase | | Kuramochi(Expasy, | | |
| | | CVCL_1345), OvCAR- | | |
| | | 432(Expasy, | | |
| | | CVCL_3769), | | |
| | | OvSAHO(Expasy | | |
| | | CVCL_3114) | | |
| Ovarian- | Ovarian | Cov318(Expasy, | Mitosis | Paclitaxel(Sigma, |
| Mitosis | | CVCL_2419), | | T7191) |
| | | Kuramochi(Expasy, | | |
| | | CVCL_1345), OvCAR- | | |
| | | 432(Expasy, | | |
| | | CVCL_3769), | | |
| | | OvSAHO(Expasy, | | |
| | | CVCL_3114) | | |
| Breast/Esopha | Breast, | MCF-7(ATCC, HTB-22), | general | radiation |
| geal-Radiation | Esophageal | T47D(ATCC, HTB-133), | | |
| | | ESO26(Expasy, | | |
| | | CVCL_2035), NCI- | | |
| | | N87(ATCC, CRL-5822), | | |
| | | OE19(ECACC, | | |
| | | 96071721) | | |
| Breast/CRC- | Breast(HER2), | HCC1954(ATCC, CRL- | Mitosis | Docetaxel(Sigma, |
| Mitosis | CRC(BRAFV600E) | 2338), SK-BR-3(ATCC, | | 01885) |
| | | HTB-30), HT29(ATCC, | | |
| | | HTB-38), | | |
| | | LS411N(ATCC, CRL- | | |
| | | 2159), RKO(ATCC, | | |
| | | CRL-2577) | | |
| Breast/CRC- | Breast(HER2), | HCC1954(ATCC, CRL- | Cell cycle | Doxorubicin(Sigma, |
| DNA synthesis | CRC(BRAFV600E) | 2338), SK-BR-3(ATCC, | (intercalation) | D1515) |
| | | HTB-30), HT29(ATCC, | | |
| | | HTB-38), | | |
| | | LS411N(ATCC, CRL- | | |
| | | 2159), RKO(ATCC, | | |
| | | CRL-2577) | | |
| Melanoma | Melanoma(BRAFV600E) | SK-MEL-5(ATCC, HTB- | MDM2, | Nutlin-3(Cayman, |
| BRAF(V600E)- | | 70), UACC62(Expasy, | CDK4/6, | 10004372), |
| various non- | | CVCL_1780) | HDAC, HMG- | Palbociclib(Sigma, |
| specific targets | | | CoA | PZ0199), |
| | | | reductase, | Vorinostat(Selleck |
| | | | HSP90 | S1047), |
| | | | | Simvastatin(Bio Vision, |
| | | | | 1693-50), 17- |
| | | | | AAG(LC Laboratories, |
| | | | | A-6880) |
| CRC(BRAFV | CRC(BRAFV600E) | HT29(ATCC, HTB-38), | BRAF, MEK | PLX4720(Chemietek |
| 600E)-BRAF, MEK | | LS411N(ATCC, CRL- | | CT-P4720), |
| | | 2159), RKO(ATCC, | | AZD6244(LC |
| | | CRL-2577) | | laboratories, S-4490) |
| Melanoma | Melanoma(BRAF wt) | 24T, 32T, 96T | MEK | Trametinib(LC |
| BRAF(wt) - | | | | Laboratories, T-8123) |
| MEK | | | | |
| Breast-ER | Breast | MCF-7(ATCC, HTB-22), | ER | Fulvestrant(Selleck, |
| | | T47D(ATCC, HTB-133) | | S1191) |
| Prostate- | Prostate | LNCaP(ATCC, CRL- | EGFR + HER2 | Lapatinib(LC |
| EGFR, HER2 | | 1740 (Clone FGC)), | | Laboratories, L-4804) |
| | | PC3(ATCC, CRL-1435) | | |
| NSCLC- | NSCLC | HCC827(ATCC, CRL- | EGFR + HER2 + | Afatinib(LC |
| EGFR/HER2 + | | 2868)(GR6) | MET + ALK | Laboratories, A- |
| MET/ALK | | | | 8644) + Crizotinib(LC |
| | | | | Laboratories, C-7900), |
| | | | | Neratinib(Selleck, |
| | | | | S2150) + Crizotinib(LC |
| | | | | Laboratories, C- |
| | | | | 7900) |
| NSCLC-EGFR + | NSCLC | HCC827(ATCC, CRL- | EGFR + MET + | Gefitinib(LC |
| MET/ALK | | 2868)(GR6) | ALK | Laboratories, G- |
| | | | | 4408) + Crizotinib(LC |
| | | | | Laboratories, C-7900), |
| | | | | Gefitinib(LC |
| | | | | Laboratories, G- |
| | | | | 4408) + WZ4002(Selleck, |
| | | | | S1173) |
| CRC(BRAFV | CRC(BRAFV600E) | RKO(ATCC, CRL-2577) | BRAF + MET + | PLX4720(Chemietek, |
| 600E)-BRAF + | | | ALK | CT-P4720) + |
| MET/ALK | | | | Crizotinib(LC |
| | | | | Laboratories, C-7900) |
| Melanoma(BR | Melanoma(BRAFV600E) | SK-MEL-28(ATCC, | BRAF + MET + | PLX4720(Chemietek, |
| AFV600E)- | | HTB-72), SK-MEL- | ALK | CT-P4720) + |
| BRAF + MET/ | | 5(ATCC, HTB-70) | | Crizotinib(LC |
| ALK | | | | Laboratories, C-7900) |
| Prostate-AR | Prostate | LNCaP(ATCC, CRL- | AR | MDV3100(Selleck, |
| | | 1740 (Clone FGC)) | | S1250) |

Animals—Experiments were approved by the Institutional Animal Care and Use Committees of the Weizmann Institute and performed in accordance with NIH guidelines. For all experiments (excluding colon xenografts) 5 weeks old athymic nude mice females were purchased from Envigo. For colon xenografts, 5 weeks NSG (NOD-Scid-Gamma) males were obtained from an in-house colony of NSG mice (originally from the The Jackson Laboratory). Littermates of the same sex were randomly assigned for the different experimental groups.

For the ex-vivo experiments EVOCs from immunocompromised mice bearing human tumors were generated, as further described hereinbelow. For the in-vivo validation experiments, mice bearing subcutaneous tumors were used; and response was measured by evaluating tumor volume. In the in-vivo experiments, mice were allocated randomly to different treatment cohorts. The investigators were not blinded to the allocation.
To model the TME effect on innate resistance to vemurafenib, the melanoma BRAF mutated cell lines G361 and UACC62 were lenti-virally infected with CMV-GFP-T2A-Luciferase (SBI, BLIV101PA-1). To image the cells in-vivo, mice were injected i.p with 15 μmg/ml D-luciferin (Caliper Life Science, #119222), 10 μl/g body weight. 10 μminutes following injection, mice were imaged with IVIS (PerkinElmer).
The following xenograft models were generated with G361 and UACC62 cell lines:
Subcutaneous tumors: 5 weeks nude mice females were injected s.c with 2×10⁶ cells in 150 μl PBS. Tumors were harvested when reaching 700 μmm³ diameter.
Liver tumors: 5 weeks nude mice females were anesthetized, and after exposure of their spleen, 2×10⁶ cells in 25 μl PBS were injected to the spleen tip. Tumors were resected from the liver based on luciferase imaging.
Lung tumors: 5 weeks nude mice female were injected i.v (tail) with 0.5×10⁶ cells in 200 μl HBSS. Tumors were resected based on luciferase imaging.
Colon tumors: 8-10 weeks NSG male mice were injected using a high resolution endoscopic system (47). 1×10⁵ cells in 50 μl PBS were injected sub-mucosal, using a custom made needle. Tumors were resected based on endoscopic imaging before bowel obstruction was reached.

High throughput in-vitro screens—For high-throughput screens or drug dose curves, cells were counted by Vi-Cell XR (Beckman coulter). Cells were seeded on 384 wells plates (Corning, 3712) using the EL406 washer dispenser (BioTek). Liquid handling of medium and drugs was effected by CyBi (WellFlex vario, CyBio). GFP fluorescence of cells was measured at 477/517 nm (excitation/emission) using Cytation3 (BioTek). Due to fluorescence reading bias at plate margins and corners, these margins were discarded for analysis.

Drug dose curves—To focus on significant secretome mediated effects on drug resistance, drugs (see Table 1 hereinabove) were used at their EC90 for blocking cell proliferation on each cell line. EC90 was measured in the following manner: On day 0, cell lines were seeded in 384 wells plates. On day 1, a gradient of drug concentrations was added, one concentration per quadruplet of wells. Thus, excluding plate margins, a 384 wells plate contained 10 drug concentrations in a pair of rows. The medium and drug were replaced on day 4. Cell fluorescence was read at days 1, 4, 6 and 7 yielding a growth curve per drug concentration per cell line. Following growth curve normalization by subtracting day 1 fluorescence, the drug concentration was selected per cell line which reduced day 7 fluorescence to 10% of the no treatment (DMSO) control level.

Assembly of the Secretome library—To assemble a collection of secreted proteins which represents the human secreteome, recombinant proteins were selected based on the secreted proteins database (SPD)(20) which contains over 4000 validated and predicted secreted proteins. Selection criteria were degree of manual curation, previous publication linking a given protein to innate resistance and commercial availability (Table 2 hereinbelow). Secretome library was organized in a 384 deep-wells plate, each well containing 155 μl of protein diluted in DMEM at 6-fold concentration of its ED50. ED50 was determined according to the literature, and corresponding references are given in Table 2 hereinbelow. Due to fluorescence reading bias at plate margins and corers, secretome plate margins were not used. In addition, control wells filled with the different factors' solvents were randomly distributed in the plate. Two versions of plate designs were used in the screens, consisting of 297/294 factors and 7/10 internal control wells, respectively. For long-term storage of the proteins stocks, proteins were reconstituted according to manufacturer instructions and stored in −80° C. at concentrations of 60×, 600×, 6000× and 60,000× the ED50 to be used, depending on the protein solubility limit. Prior to secretome screen experiments, each factor was diluted to 6X ED50 concentration in 155 μl DMEM, and organized in 384 deep wells plate using the CyBi liquid handler.

TABLE 2
Secretome library

| | | | cat. | | | | ED50 | Max | |
|---|---|---|---|---|---|---|---|---|---|
| | | HGNC | (Sigma unless | | ED50 | | to use | Reconstitution | |
| | Gene | official name: | specified else) | Type | (ng/ml) | | (ng/ml) | (ug/mL) | Solution |
| 1 | ACHE | acetylcholinesterase | C0663 | Units/ | 4 | units/mL | 0.03 | supplied as | PBS |
| | | | | Solution | | | (0.5 U/ml) | solution | |
| 2 | AHSG | alpha-2- | G0516 | Normal | 20000 | | 10000 | 500 | 20 mM |
| | | HS- | | | | | | | Tris- |
| | | glycoprotein | | | | | | | HCl, |
| | | | | | | | | | pH 8 |
| 3 | ANG | angiogenin, | A6955 | Normal | 200-2000 | | 500 | >50 | PBS w |
| | | ribonuclease, | | | | | | | 0.1% |
| | | RNase A | | | | | | | BSA |
| | | family, 5 | | | | | | | |
| 4 | ANGPT1 | angiopoietin 1 | SRP3007 | Normal | 10-40.0 | | 50 | 1000 | PBS w |
| | | | | | | | | | 0.1% |
| | | | | | | | | | BSA |
| 5 | ANGPT2 | angiopoietin 2 | A9847 | Normal | 200 | | 200 | 100 | PBS w |
| | | | | | | | | | 0.1% |
| | | | | | | | | | BSA |
| 6 | ANGPT4 | angiopoietin 4 | A1479 | Normal | 40 | | 50 | >10 | PBS w |
| | | | | | | | | | 0.1% |
| | | | | | | | | | BSA |
| 7 | APCS | amyloid P | S5269 | Solution | 2000 | | 2000 | Supplied in | PBS w |
| | | component, | | | | | | solution | 0.1% |
| | | serum | | | | | | | BSA |
| 8 | APOA4 | apolipoprotein | L1567 | Normal | plasma: 151000 | | 100000 | 1000 | DDW |
| | | A-IV | | | | | | | |
| 9 | ARTN | artemin | SRP4515 | Normal | 4-8 | | 100 | 1000 | PBS w |
| | | | | | | | | | 0.1% |
| | | | | | | | | | BSA |
| 10 | B2M | beta-2- | M4890 | Normal | 800-2400 | | 1000 | >100 | DDW |
| | | microglobulin | | | | | | | |
| 11 | BDNF | brain- | B3795 | Normal | 25-50 | | 50 | 1000 | DDW |
| | | derived | | | | | | | |
| | | neurotrophic | | | | | | | |
| | | factor | | | | | | | |
| 12 | GDF11 | bone | SRP4576 | Normal | 2-900 | | 100 | 1000 | PBS w |
| | | morphogenetic | | | | | | | 0.1% |
| | | protein 11 | | | | | | | BSA |
| 13 | BTC | betacellulin | B3670 | Normal | 0.5 | | 100 | 100 | PBS w |
| | | | | | | | | | 0.1% |
| | | | | | | | | | BSA |
| 14 | CCL13 | chemokine (C-C | M246 | Normal | 200-600 | | 200 | >10 | PBS w |
| | | motif) ligand 13 | | | | | | | 0.1% |
| | | | | | | | | | BSA |
| 15 | CCL5 | chemokine (C-C | R6267 | Normal | 1.0-5.0 | | 100 | 100 | PBS w |
| | | motif) ligand 5 | | | | | | | 0.1% |
| | | | | | | | | | BSA |
| 16 | CSF1 | colony | SRP4237 | Normal | 0.5-5 | | 100 | 500 | PBS w |
| | | stimulating | | | | | | | 0.1% |
| | | factor 1 | | | | | | | BSA |
| | | (macrophage) | | | | | | | |
| 17 | CSF3 | colony | G0407 | Normal | 0.01-0.1 | | 50 | >1 | PBS w |
| | | stimulating | | | | | | | 0.1% |
| | | factor 3 | | | | | | | BSA |
| | | (granulocyte) | | | | | | | |
| 18 | CXCL10 | chemokine (C-X-C | I3400 | Normal | 0.087 | | 100 | 20 | PBS w |
| | | motif) ligand 10 | | | | | | | 0.1% |
| | | | | | | | | | BSA |
| 19 | CXCL12 | chemokine (C-X-C | S190 | Normal | 0.18 | | 100 | 100 | PBS w |
| | | motif) ligand 12 | | | | | | | 0.1% |
| | | | | | | | | | BSA |
| 20 | EGFR | epidermal | E3641 | Units/ | 50 | U/mL | 0.3 | supplied as | 10% |
| | | growth factor | | Solution | | | (5 U/ml) | solution | glycerol |
| | | receptor | | | | | | | |
| 21 | ELANE | elastase, | A6150 | Normal | 50000 | | 50000 | 1000 | PBS |
| | | neutrophil | | | | | | | |
| | | expressed | | | | | | | |
| 22 | F2 | coagulation | T1063 | Units | 0.0575 | U/mL | 0.15 | 10000 U/ml | DDW |
| | | factor II | | | | | (2.5 U/ml) | | |
| | | (thrombin) | | | | | | | |
| 23 | FGF21 | fibroblast | SRP4066 | Normal | 120-600 | | 100 | 100 | PBS w |
| | | growth factor 21 | | | | | | | 0.1% |
| | | | | | | | | | BSA |
| 24 | FGF22 | fibroblast | SRP4063 | Normal | 50-300 | | 100 | 100 | PBS w |
| | | growth factor 22 | | | | | | | 0.1% |
| | | | | | | | | | BSA |
| 25 | FGFR2 | fibroblast | SRP5030 | Solution | 15-30 | | 100 | supplied as | PBS w |
| | | growth factor | | | | | | solution | 0.1% |
| | | receptor 2 | | | | | | | BSA |
| 26 | FIGF | c-fos induced | V6012 | Normal | 8-500 | | 50 | 100 | PBS w |
| | | growth factor | | | | | | | 0.1% |
| | | (vascular | | | | | | | BSA |
| | | endothelial | | | | | | | |
| | | growth factor D) | | | | | | | |
| 27 | FN1 | fibronectin 1 | F2006 | Normal | 500-50000 | | 10000 | 1000 | DDW |
| 28 | GAST | gastrin | G1260 | Normal | 1000 | | 1000 | 100 | PBS |
| 29 | HGF | hepatocyte | H1404 | Normal | 20-40 | | 50 | 100 | DDW |
| | | growth factor | | | | | | | |
| | | (hepapoietin A; | | | | | | | |
| | | scatter factor) | | | | | | | |
| 30 | IL16 | interleukin 16 | SRP3079 | Normal | 50-1000 | | 100 | 5 | PBS w |
| | | | | | | | | | 0.1% |
| | | | | | | | | | BSA |
| 31 | IL20 | interleukin 20 | SRP4548 | Normal | 0.2-0.6 | | 100 | 1000 | PBS w |
| | | | | | | | | | 0.1% |
| | | | | | | | | | BSA |
| 32 | KITLG | kit ligand | S7901 | Normal | 2.5-10 | | 50 | 50 | PBS w |
| | | (=stem cell | | | | | | | 0.1% |
| | | factor) | | | | | | | BSA |
| 33 | KNG | Kininostatin | B3259 | Normal | 200-1000 | | 1000 | 25 | PBS w |
| | | | | | | | | | 0.1% |
| | | | | | | | | | BSA |
| 34 | LAMA1 | laminin, | L6274 | Solution | 5000 | | 5000 | supplied as | DDW |
| | | alpha 1 | | | | | | solution | |
| 35 | MSMB | microseminoprotein, | I1398 | Normal | 1000 | | 1000 | 100 | PBS |
| | | beta- | | | | | | | |
| 36 | MSTN | myostatin | SRP4623P | Normal | 2-100 | | 100 | 100 | 4 mM |
| | | | | | | | | | HCl w |
| | | | | | | | | | 0.1% |
| | | | | | | | | | BSA |
| 37 | NRP1 | neuropilin 1 | SRP3126 | Normal | 1-10.0 | | 100 | 100 | PBS |
| 38 | NRP2 | neuropilin 2 | SRP4363 | Normal | 1-7.0 | | 100 | 100 | PBS |
| 39 | NTF4 | neurotrophin 4 | N1780 | Normal | 0.3-3 | | 50 | 50 | PBS w |
| | | | | | | | | | 0.1% |
| | | | | | | | | | BSA |
| 40 | OSM | oncostatin M | SRP3130 | Normal | 0.05-2 | | 100 | 100 | PBS w |
| | | | | | | | | | 0.1% |
| | | | | | | | | | BSA |
| 41 | PPBP | pro-platelet | SRP3121 | Normal | 1-10.0 | | 50 | 50 | PBS w |
| | | basic protein | | | | | | | 0.1% |
| | | (chemokine (C-X-C | | | | | | | BSA |
| | | motif) ligand 7) | | | | | | | |
| 42 | SERPINA3 | serpin peptidase | A9285 | Normal | 355, 8 nM | | 400 | 100 | 20 mM |
| | | inhibitor, | | | | | | | Tris- |
| | | clade A (alpha-1 | | | | | | | HCl, |
| | | antiproteinase, | | | | | | | pH 8 |
| | | antitrypsin), | | | | | | | |
| | | member 3 | | | | | | | |
| 43 | THPO | thrombopoietin | T1568 | Normal | 0.3-3 | | 50 | 50 | PBS w |
| | | | | | | | | | 0.1% |
| | | | | | | | | | BSA |
| 44 | TIMP1 | TIMP | SRP3173 | Normal | 500 | | 100 | 100-1000 | DDW |
| | | metallopeptidase | | | | | | | |
| | | inhibitor 1 | | | | | | | |
| 45 | TIMP2 | TIMP | SRP3174 | Normal | 500 | | 100 | 100-1000 | DDW |
| | | metallopeptidase | | | | | | | |
| | | inhibitor 2 | | | | | | | |
| 46 | TNFRSF18 | tumor necrosis | G1667 | Normal | 2000 | | 1000 | 100 | PBS |
| | | factor receptor | | | | | | | |
| | | superfamily, | | | | | | | |
| | | member 18 | | | | | | | |
| 47 | VTN | vitronectin | SRP3186 | Normal | 5000 | | 2500 | 1000 | PBS |
| 48 | WNT1 | wingless- | SRP4754 | Normal | 1.5-2.5 | | 100 | 1000 | DDW |
| | | type MMTV | | | | | | | |
| | | integration | | | | | | | |
| | | site family, | | | | | | | |
| | | member 1 | | | | | | | |
| 49 | APLN | apelin | A6469 | Normal | 0.2-0.4 | | 5 | 10000 | PBS |
| 50 | AVP | arginine | V9879 | Normal | Plasma: 0.01 | | 5 | 20000 | DDW |
| | | vasopressin | | | | | | | |
| 51 | BGLAP | bone gamma- | O5761 | Normal | 7 | | 20 | 1000 | 100 mM |
| | | carboxyglutamate | | | | | | | Sodium |
| | | (gla) protein | | | | | | | Bicarbonate |
| 52 | CMA1 | chymase 1, | C8118 | Solution | 0.454 | | 2 | supplied in | 20 mM |
| | | mast cell | | | | | | solution | Tris- |
| | | | | | | | | | HCl, pH |
| | | | | | | | | | 8.0 w |
| | | | | | | | | | 0.1% |
| | | | | | | | | | BSA |
| 53 | EFNB3 | ephrin-B3 | E0903 | Normal | 0.08-5 | | 10 | 1000 | PBS |
| 54 | EGF | epidermal | E9644 | Normal | 0.02-0.1 | | 10 | 500 | PBS |
| | | growth factor | | | | | | | |
| 55 | ELN | elastin | E7277 | Normal | plasma: 23.4-66.8 | | 150 | 10000 | DDW |
| 56 | EREG | epiregulin | SRP3033 | Normal | <0.2 | | 0.2 | 1000 | PBS w |
| | | | | | | | | | 0.1% |
| | | | | | | | | | BSA |
| 57 | GNRH1 | gonadotrop | L7134 | Normal | 0.1 | | 10 | 25000 | DDW |
| | | in-releasing | | | | | | | |
| | | hormone 1 | | | | | | | |
| | | (luteinizing- | | | | | | | |
| | | releasing hormone) | | | | | | | |
| 58 | IFNA1 | interferon, alpha 1 | SRP4596 | Normal | 0.05 | | 1 | 1000 | PBS w |
| | | | | | | | | | 0.1% |
| | | | | | | | | | BSA |
| 59 | IFNA13 | interferon, alpha 13 | I4401 | Units/ | 5 | U/ml | 0.5 | supplied as | PBS w |
| | | | | Solution | | | (8.33 U/ml) | solution | 0.1% |
| | | | | | | | | | BSA |
| 60 | IFNA2 | interferon, alpha 2 | SRP4594 | Normal | 0.05 | | 1 | 1000 | PBS w |
| | | | | | | | | | 0.1% |
| | | | | | | | | | BSA |
| 61 | IFNB1 | interferon, beta 1, | I4151 | Units/ | 1 | U/ml | 0.1 | supplied as | PBS w |
| | | | | Solution | | | (1.66 U/ml) | solution | 0.1% |
| | | fibroblast | | | | | | | BSA |
| 62 | IFNW1 | interferon, | SRP3061 | Normal | 0.01 | | 1 | 1000 | PBS w |
| | | omega 1 | | | | | | | 0.1% |
| | | | | | | | | | BSA |
| 63 | IGF1 | insulin-like | SRP3069 | Normal | 2 | | 5 | 1000 | PBS |
| | | growth factor 1 | | | | | | | |
| | | (somatomedin C) | | | | | | | |
| 64 | MMP7 | matrix | M4565 | Solution | 0.81-3.22 | | 1 | supplied in | PBS |
| | | metallopeptidase | | | | | | solution | |
| | | 7 (matrilysin, | | | | | | | |
| | | uterine) | | | | | | | |
| 65 | NPPA | natriuretic | A1663 | Normal | 0.042 | | 1 | 1000 | DDW |
| | | peptide A | | | | | | | |
| 66 | NPPC | natriuretic | N8768 | Normal | plasma: 0.004 | | 1 | 1000 | DDW |
| | | peptide C | | | | | | | |
| 67 | NPY | neuropeptide Y | N5017 | Normal | plasma: 0.303-1.211 | | 2 | 1000 | DDW |
| 68 | OXT | oxytocin, | 06379 | Normal | | | 2 | 10000 | DDW |
| | | prepropeptide | | | | | | | |
| 69 | PNOC | prepronociceptin | N0524 | Normal | plasma: 0.01 | | 1 | 2000 | DDW |
| 70 | TF | transferrin | T3309 | Normal | 75-375 | | 400 | 50000 | DDW |
| 71 | UCN | urocortin | U4127 | Normal | 1.76 +/− 0.84 | | 20 | 1000 | 10 mM |
| | | | | | | | | | acetic |
| | | | | | | | | | acid |
| 72 | UCN3 | urocortin 3 | U1008 | Normal | 0.5-100 | | 20 | 1000 | 10 mM |
| | | (stresscopin) | | | | | | | acetic |
| | | | | | | | | | acid |
| 73 | UTS2 | urotensin 2 | U7257 | Normal | 0.138; 0.1 nM | | 5 | 1000 | DDW |
| 74 | AGT | angiotensinogen | A2562 | Normal | Plasma: 146-2457 | | 500 | 25000 | DDW |
| | | (serpin | | | | | | | |
| | | peptidase | | | | | | | |
| | | inhibitor, | | | | | | | |
| | | clade A, | | | | | | | |
| | | member 8) | | | | | | | |
| 75 | EMAPII | aminoacyl | SRP4463 | Normal | 20-40 | | 50 | 100-1000 | DDW |
| | | tRNA | | | | | | | |
| | | synthetase | | | | | | | |
| | | complex- | | | | | | | |
| | | interacting | | | | | | | |
| | | multifunctional | | | | | | | |
| | | protein 1 | | | | | | | |
| 76 | APOE | apolipoprotein E | SRP4760 | Normal | 1000 | | 1000 | 1000 | DDW |
| 77 | BMP10 | bone | SRP4581 | Normal | 15-3000 | | 50 | 1000 | PBS w |
| | | morphogenetic | | | | | | | 0.1% |
| | | protein 10 | | | | | | | BSA |
| 78 | GDF6 | bone | SRP4639 | Normal | 2000-3000 | | 500 | 1000 | PBS w |
| | | morphogenetic | | | | | | | 0.1% |
| | | protein 13 | | | | | | | BSA |
| 79 | BMP2 | bone | B3555 | Normal | 40-1000 | | 50 | 1000 | PBS w |
| | | morphogenetic | | | | | | | 0.1% |
| | | protein 2 | | | | | | | BSA |
| 80 | BMP4 | bone | B2680 | Normal | 10-30 | | 50 | 1000 | PBS w |
| | | morphogenetic | | | | | | | 0.1% |
| | | protein 4 | | | | | | | BSA |
| 81 | BMP6 | bone | B2805 | Normal | 50-150 | | 50 | 1000 | PBS w |
| | | morphogenetic | | | | | | | 0.1% |
| | | protein 6 | | | | | | | BSA |
| 82 | BMP7 | bone | B1434 | Normal | 100-600 | | 100 | 1000 | PBS w |
| | | morphogenetic | | | | | | | 0.1% |
| | | protein 7 | | | | | | | BSA |
| 83 | CCL1 | chemokine (C-C | I152 | Normal | 30-60 | | 50 | >10 | PBS w |
| | | motif) ligand 1 | | | | | | | 0.1% |
| | | | | | | | | | BSA |
| 84 | CCL14 | chemokine (C-C | H0656 | Normal | 200-15000 | | 100 | >100 | PBS w |
| | | motif) ligand 14 | | | | | | | 0.1% |
| | | | | | | | | | BSA |
| 85 | CCL15 | chemokine (C-C | SRP4260 | Normal | 2-800 | | 200 | 1000 | PBS w |
| | | motif) ligand 15 | | | | | | | 0.1% |
| | | | | | | | | | BSA |
| 86 | CCL16 | chemokine (C-C | SRP3105 | Normal | 10-100 | | 100 | 1000 | PBS w |
| | | motif) ligand 16 | | | | | | | 0.1% |
| | | | | | | | | | BSA |
| 87 | CCL18 | chemokine (C-C | SRP4257 | Normal | 100 | | 100 | 1000 | PBS w |
| | | motif) ligand 18 | | | | | | | 0.1% |
| | | (pulmonary and | | | | | | | |
| | | activation- | | | | | | | |
| | | regulated) | | | | | | | |
| 88 | CCL2 | chemokine (C-C | M6667 | Normal | 8 | | 15 | 100 | PBS w |
| | | motif) ligand 2 | | | | | | | 0.1% |
| | | | | | | | | | BSA |
| 89 | CCL20 | chemokine (C-C | SRP4491 | Normal | 0.5-70.0 | | 50 | 1000 | PBS w |
| | | motif) ligand 20 | | | | | | | 0.1% |
| | | | | | | | | | BSA |
| 90 | CCL21 | chemokine (C-C | SRP3035 | Normal | 10-100 | | 100 | 1000 | PBS w |
| | | motif) ligand 21 | | | | | | | 0.1% |
| | | | | | | | | | BSA |
| 91 | CCL23 | chemokine (C-C | SRP3116 | Normal | 10-50 | | 50 | 1000 | PBS w |
| | | motif) ligand 23 | | | | | | | 0.1% |
| | | | | | | | | | BSA |
| 92 | CCL24 | chemokine (C-C | SRP4497 | Normal | 10-50 | | 50 | 1000 | PBS w |
| | | motif) ligand 24 | | | | | | | 0.1% |
| | | | | | | | | | BSA |
| 93 | CCL25 | chemokine (C-C | SRP3168 | Normal | 1-12000 | | 200 | 1000 | PBS w |
| | | motif) ligand 25 | | | | | | | 0.1% |
| | | | | | | | | | BSA |
| 94 | CCL26 | chemokine (C-C | E8399 | Normal | 200-1000 | | 200 | >25 | PBS w |
| | | motif) ligand 26 | | | | | | | 0.1% |
| | | | | | | | | | BSA |
| 95 | CCL28 | chemokine (C-C | SRP3112 | Normal | 1-2000 | | 200 | 1000 | PBS w |
| | | motif) ligand 28 | | | | | | | 0.1% |
| | | | | | | | | | BSA |
| 96 | CCL3 | chemokine (C-C | M6292 | Normal | 2-11 | | 15 | 100 | PBS w |
| | | motif) ligand 3 | | | | | | | 0.1% |
| | | | | | | | | | BSA |
| 97 | CCL4 | chemokine (C-C | M6417 | Normal | 3.0-30.0 | | 15 | 100 | PBS w |
| | | motif) ligand 4 | | | | | | | 0.1% |
| | | | | | | | | | BSA |
| 98 | CCL7 | chemokine (C-C | M8543 | Normal | 20-500 | | 100 | >10 | PBS w |
| | | motif) ligand 7 | | | | | | | 0.1% |
| | | | | | | | | | BSA |
| 99 | CFI | complement | C5938 | Normal | plasma: 1000 | | 1000 | 1000 | PBS |
| | | factor I | | | | | | | |
| 100 | CLEC11A | C-type lectin | SRP3152 | Normal | 2.5 | | 20 | 100 | PBS w |
| | | domain family 11, | | | | | | | 0.1% |
| | | member A | | | | | | | BSA |
| 101 | CNTF | ciliary | C3710 | Normal | 50-150 | | 100 | >25 | PBS w |
| | | neurotrophic | | | | | | | 0.1% |
| | | factor | | | | | | | BSA |
| 102 | COL18A1 | collagen, type | SRP3031 | Normal | 500-1000 | | 800 | 1000 | PBS w |
| | | XVIII, alpha 1 | | | | | | | 0.1% |
| | | | | | | | | | BSA |
| 103 | COL1A2 | collagen, type | C5483 | Normal | 1000 | | 400 | 1000 | PBS w |
| | | I, alpha 2 | | | | | | | 0.1% |
| | | | | | | | | | BSA |
| 104 | COL4A1 | collagen, type | C6745 | Normal | 1000 | | 800 | 1000 | PBS w |
| | | IV, alpha 1 | | | | | | | 0.1% |
| | | | | | | | | | BSA |
| 105 | COL5A1 | collagen, type | C5983 | Normal | 1000 | | 400 | 1000 | PBS w |
| | | V, alpha 1 | | | | | | | 0.1% |
| | | | | | | | | | BSA |
| 106 | COL9A1 | collagen, type | C3657 | Normal | 1000 | | 400 | 1000 | PBS w |
| | | IX, alpha 1 | | | | | | | 0.1% |
| | | | | | | | | | BSA |
| 107 | CRP | C-reactive | C1617 | Solution | 150-10000 | | 1000 | 1000 | 20 mM |
| | | protein, | | | | | | | Tris- |
| | | pentraxin- | | | | | | | HCl, |
| | | related | | | | | | | pH 8.0 w |
| | | | | | | | | | 0.1% |
| | | | | | | | | | BSA |
| 108 | CSH1 | chorionic | SRP4869 | Normal | 0.1-0.5 | | 25 | 1000 | PBS w |
| | | somatomammotropin | | | | | | | 0.1% |
| | | hormone 1 | | | | | | | BSA |
| | | (placental | | | | | | | |
| | | lactogen) | | | | | | | |
| 109 | CTF1 | cardiotrophin 1 | SRP4011 | Normal | 0.25-1.25 | | 25 | <1000 | 4mM |
| | | | | | | | | | HCl w |
| | | | | | | | | | 0.1% |
| | | | | | | | | | BSA |
| 110 | CTRB1 | chymotrypsinogen | C8946 | Normal | 1020 | | 500 | 2000 | 1 mM |
| | | B1 | | | | | | | HCl |
| 111 | CTSS | cathepsin S | C5993 | Normal | 5 | | 50 | 1000 | DDW |
| 112 | CX3CL1 | chemokine (C-X3-C | F1300 | Normal | 0.3-100 | | 50 | >50 | PBS w |
| | | motif) ligand 1 | | | | | | | 0.1% |
| | | | | | | | | | BSA |
| 113 | CXCL1 | chemokine (C-X-C | G0657 | Normal | 2.0-5.0 | | 50 | 100 | 20 mM |
| | | motif) ligand 1 | | | | | | | Tris- |
| | | (melanoma growth | | | | | | | HCl, |
| | | stimulating | | | | | | | pH 8.0 w |
| | | activity, alpha) | | | | | | | 0.1% |
| | | | | | | | | | BSA |
| 114 | CXCL16 | chemokine (C-X-C | SRP3023 | Normal | 1-100 | | 100 | 1000 | PBS w |
| | | motif) ligand 16 | | | | | | | 0.1% |
| | | | | | | | | | BSA |
| 115 | CXCL3 | chemokine (C-X-C | SRP4107 | Normal | 10-100.0 | | 80 | 1000 | PBS w |
| | | motif) ligand 3 | | | | | | | 0.1% |
| | | | | | | | | | BSA |
| 116 | CXCL5 | chemokine (C-X-C | SRP4025 | Normal | 50 | | 50 | 1000 | PBS w |
| | | motif) ligand 5 | | | | | | | 0.1% |
| | | | | | | | | | BSA |
| 117 | CXCL6 | chemokine (C-X-C | G150 | Normal | 3-1500 | | 25 | 10 | PBS w |
| | | motif) ligand 6 | | | | | | | 0.1% |
| | | (granulocyte | | | | | | | BSA |
| | | chemotactic | | | | | | | |
| | | protein 2) | | | | | | | |
| 118 | CXCL9 | chemokine (C-X-C | M252 | Normal | 100-400 | | 100 | 100 | PBS w |
| | | motif) ligand 9 | | | | | | | 0.1% |
| | | | | | | | | | BSA |
| 119 | DKK1 | dickkopf 1 | SRP3258 | Normal | 200 | | 100 | 1000 | PBS w |
| | | homolog | | | | | | | 0.1% |
| | | (Xenopus laevis) | | | | | | | BSA |
| 120 | EPGN | epithelial | SRP4969 | Normal | 100-500 | | 100 | 100 | PBS w |
| | | mitogen homolog | | | | | | | 0.1% |
| | | (mouse) | | | | | | | BSA |
| 121 | EPHA3 | EPH | E7409 | Normal | 5-25.0 | | 25 | 100 | PBS |
| | | receptor A3 | | | | | | | |
| 122 | EPO | erythropoietin | E5627 | Units | 0.015-0.075 | U/mL | 0.0045 | >500 U/ml | PBS w |
| | | | | | | | (0.075 U/ml) | | 0.1% |
| | | | | | | | | | BSA |
| 123 | F5 | coagulation | F0931 | Solution | 500 | | 400 | supplied in | PBS |
| | | factor V | | | | | | solution | |
| | | (proaccelerin, | | | | | | | |
| | | labile factor) | | | | | | | |
| 124 | FGB | fibrinogen | F3879 | Normal | plasma: 2000000-4000000 | | 200000 | | PBS |
| | | beta chain | | | | | | | |
| 125 | FGF1 | fibroblast | SRP2091 | Normal | 0.1-0.3 | | 25 | 25 | PBS w |
| | | growth factor 1 | | | | | | | 0.1% |
| | | (acidic) | | | | | | | BSA |
| 126 | FGF10 | fibroblast | F8924 | Normal | 20-100 | | 100 | 100 | PBS w |
| | | growth factor 10 | | | | | | | 0.1% |
| | | | | | | | | | BSA |
| 127 | FGF17 | fibroblast | F7176 | Normal | 15-500 | | 100 | >25 | PBS w |
| | | growth factor 17 | | | | | | | 0.1% |
| | | | | | | | | | BSA |
| 128 | FGF19 | fibroblast | SRP4542 | Normal | 3-200 | | 50 | 1000 | PBS w |
| | | growth factor 19 | | | | | | | 0.1% |
| | | | | | | | | | BSA |
| 129 | FGF20 | fibroblast | SRP4589 | Normal | 0.2-10 | | 20 | 1000 | PBS w |
| | | growth factor 20 | | | | | | | 0.1% |
| | | | | | | | | | BSA |
| 130 | FGF23 | fibroblast | SRP3039 | Normal | 100-400 | | 150 | 500 | PBS w |
| | | growth factor 23 | | | | | | | 0.1% |
| | | | | | | | | | BSA |
| 131 | FGF6 | fibroblast | F4662 | Normal | 0.1-0.3 | | 25 | 10 | PBS w |
| | | growth factor 6 | | | | | | | 0.1% |
| | | | | | | | | | BSA |
| 132 | FGF7 | fibroblast | SRP3100 | Normal | 10-75 | | 20 | 100 | PBS w |
| | | growth factor 7 | | | | | | | 0.1% |
| | | | | | | | | | BSA |
| 133 | FGF9 | fibroblast | SRP3040 | Normal | 0.5 | | 25 | 1000 | PBS w |
| | | growth factor 9 | | | | | | | 0.1% |
| | | (glia-activating | | | | | | | BSA |
| | | factor) | | | | | | | |
| 134 | FLT3LG | fms-related | SRP3044 | Normal | 1 | | 25 | 1000 | PBS w |
| | | tyrosine | | | | | | | 0.1% |
| | | kinase 3 | | | | | | | BSA |
| | | ligand | | | | | | | |
| 135 | FSHB | follicle | F4021 | Normal | 0.06-0.48 | | 15 | 100 | PBS w |
| | | stimulating | | | | | | | 0.1% |
| | | hormone, | | | | | | | BSA |
| | | beta | | | | | | | |
| | | polypeptide | | | | | | | |
| 136 | FST | follistatin | F1175 | Normal | 100-400 | | 100 | >10 | PBS w |
| | | | | | | | | | 0.1% |
| | | | | | | | | | BSA |
| 137 | GAS6 | growth arrest- | 885-GS-050 | Normal | 100-400 | | 100 | 100 | DDW |
| | | specific 6 | (R&D) | | | | | | |
| 138 | GC | group-specific | G8764 | Normal | 3000 | | 3000 | 2000 | PBS w |
| | | component | | | | | | | 0.1% |
| | | (vitamin D | | | | | | | BSA |
| | | binding protein) | | | | | | | |
| 139 | GDF3 | growth | SRP4757 | Normal | 50 | | 50 | 100 | PBS w |
| | | differentiation | | | | | | | 0.1% |
| | | factor 3 | | | | | | | BSA |
| 140 | GDF5 | growth | SRP4580 | Normal | 500-4000 | | 100 | 1000 | PBS w |
| | | differentiation | | | | | | | 0.1% |
| | | factor 5 | | | | | | | BSA |
| 141 | Gdf7 | growth | SRP4572 | Normal | 250-8000 | | 200 | 1000 | PBS w |
| | | differentiation | | | | | | | 0.1% |
| | | factor 7 | | | | | | | BSA |
| 142 | GDF9 | growth | SRP4872 | Normal | 100 | | 100 | 100 | PBS w |
| | | differentiation | | | | | | | 0.1% |
| | | factor 9 | | | | | | | BSA |
| 143 | GDNF | glial cell | G1777 | Normal | 5.0-10.0 | | 25 | 1000 | PBS w |
| | | derived | | | | | | | 0.1% |
| | | neurotrophic | | | | | | | BSA |
| | | factor | | | | | | | |
| 144 | GREM2 | gremlin 2 | SRP4657 | Normal | 150-750 | | 200 | 1000 | DDW |
| 145 | HF1 | Complement | C5813 | Normal | 2500-10000 | | 1000 | 1000 | PBS |
| | | Factor H | | | | | | | |
| 146 | HGF | Hepatocyte | 228-10702-2 | Normal | 10-100 | | 50 | 100 | DDW |
| | (raybiotech) | Growth Factor | (Ray Biotech) | | | | | | |
| 147 | IGFBP1 | insulin-like | SRP3062 | Normal | 500-4000 | | 250 | 100 | PBS |
| | | growth factor | | | | | | | |
| | | binding protein 1 | | | | | | | |
| 148 | IGFBP2 | insulin-like | SRP3063 | Normal | 30-90 | | 100 | 100 | PBS |
| | | growth factor | | | | | | | |
| | | binding protein 2, | | | | | | | |
| | | 36 kDa | | | | | | | |
| 149 | IGFBP3 | insulin-like | SRP3067 | Normal | 50-200 | | 100 | 100 | PBS |
| | | growth factor | | | | | | | |
| | | binding protein 3 | | | | | | | |
| 150 | IGFBP4 | insulin-like | SRP3064 | Normal | 30-90 | | 100 | 100 | PBS |
| | | growth factor | | | | | | | |
| | | binding protein 4 | | | | | | | |
| 151 | IGFBP5 | insulin-like | SRP3068 | Normal | 300-1500 | | 250 | 1000 | PBS |
| | | growth factor | | | | | | | |
| | | binding protein 5 | | | | | | | |
| 152 | IGFBP6 | insulin-like | SRP3065 | Normal | 100-2000 | | 200 | 200 | PBS |
| | | growth factor | | | | | | | |
| | | binding protein 6 | | | | | | | |
| 153 | IL10 | interleukin 10 | SRP3071 | Normal | 0.15-2 | | 10 | 1000 | PBS w |
| | | | | | | | | | 0.1% |
| | | | | | | | | | BSA |
| 154 | IL11 | interleukin 11 | SRP3072 | Normal | 0.2-2 | | 10 | 1000 | PBS w |
| | | | | | | | | | 0.1% |
| | | | | | | | | | BSA |
| 155 | IL12B | interleukin 12 | SRP3073 | Normal | 0.01-1 | | 10 | 1000 | PBS w |
| | | | | | | | | | 0.1% |
| | | | | | | | | | BSA |
| 156 | IL12B | interleukin 12B | I2276 | Normal | 0.05 | | 10 | >1 | PBS w |
| | | (natural | | | | | | | 0.1% |
| | | killer cell | | | | | | | BSA |
| | | stimulatory | | | | | | | |
| | | factor 2, | | | | | | | |
| | | cytotoxic | | | | | | | |
| | | lymphocyte | | | | | | | |
| | | maturation | | | | | | | |
| | | factor 2, p40) | | | | | | | |
| 157 | IL13 | interleukin 13 | SRP3076 | Normal | 0.75-3.5 | | 25 | 1000 | PBS w |
| | | | | | | | | | 0.1% |
| | | | | | | | | | BSA |
| 158 | IL15 | interleukin 15 | SRP3077 | Normal | 0.5-4 | | 25 | 50 | PBS w |
| | | | | | | | | | 0.1% |
| | | | | | | | | | BSA |
| 159 | IL17B | interleukin 17B | SRP3081 | Normal | 10-100 | | 50 | 1000 | DDW |
| 160 | IL19 | interleukin 19 | SRP4545 | Normal | 0.5-1.5 | | 10 | 1000 | PBS w |
| | | | | | | | | | 0.1% |
| | | | | | | | | | BSA |
| 161 | IL1A | interleukin 1, | I2778 | Normal | 0.001-0.006 | | 5 | 10 | PBS w |
| | | alpha | | | | | | | 0.1% |
| | | | | | | | | | BSA |
| 162 | IL2 | interleukin 2 | I2644 | Normal | 0.05-0.25 | | 20 | 100 | 100 mM |
| | | | | | | | | | Acetic |
| | | | | | | | | | Acid w |
| | | | | | | | | | 0.1% |
| | | | | | | | | | BSA |
| 163 | NRG1 | neuregulin 1 | SRP3055 | Normal | 20-100 | | 100 | 100 | PBS |
| 164 | IL22 | interleukin 22 | SRP3089 | Normal | 0.06-0.3 | | 25 | 1000 | PBS w |
| | | | | | | | | | 0.1% |
| | | | | | | | | | BSA |
| 165 | IL24 | interleukin 24 | SRP4975 | Normal | 0.1-1 | | 25 | >100 | PBS w |
| | | | | | | | | | 0.1% |
| | | | | | | | | | BSA |
| 166 | IFNL2 | interleukin 28A | SRP3060 | Normal | 10-50.0 | | 25 | 1000 | DDW |
| | | (interferon, | | | | | | | |
| | | lambda 2) | | | | | | | |
| 167 | IL29 | interleukin 29 | SRP3059 | Normal | 1-5.0 | | 25 | 1000 | DDW |
| | | (interferon, | | | | | | | |
| | | lambda 1) | | | | | | | |
| 168 | IL31 | interleukin 31 | SRP3091 | Normal | 5 | | 25 | 1000 | PBS w |
| | | | | | | | | | 0.1% |
| | | | | | | | | | BSA |
| 169 | IL4 | interleukin 4 | SRP4137 | Normal | 0.05-0.2 | | 25 | 100 | PBS w |
| | | | | | | | | | 0.1% |
| | | | | | | | | | BSA |
| 170 | IL5 | interleukin 5 | I5273 | Normal | 0.04-0.5 | | 10 | 50 | PBS w |
| | | (colony- | | | | | | | 0.1% |
| | | stimulating | | | | | | | BSA |
| | | factor, | | | | | | | |
| | | eosinophil) | | | | | | | |
| 171 | IL6 | interleukin 6 | SRP3096 | Normal | 0.1-0.8 | | 25 | 100 | PBS w |
| | | (interferon, | | | | | | | 0.1% |
| | | beta 2) | | | | | | | BSA |
| 172 | IL7 | interleukin 7 | I5896 | Normal | 0.2-0.5 | | 25 | 50 | PBS w |
| | | | | | | | | | 0.1% |
| | | | | | | | | | BSA |
| 173 | IL8 | interleukin 8 | SRP3098 | Normal | 25-150 | | 150 | 100 | PBS w |
| | | | | | | | | | 0.1% |
| | | | | | | | | | BSA |
| 174 | IL9 | interleukin 9 | I3394 | Normal | 0.1-0.6 | | 25 | 100 | PBS w |
| | | | | | | | | | 0.1% |
| | | | | | | | | | BSA |
| 175 | INHBA | inhibin, | A4941 | Normal | 0.2-1.2 | | 10 | 50 | PBS w |
| | | beta A | | | | | | | 0.1% |
| | | | | | | | | | BSA |
| 176 | INS | insulin | 19278 | Solution | 5000-10000 | | 8300.0 | supplied in | PBS |
| | | | | | | | | solution | |
| 177 | KLK1 | kallikrein 1 | K2638 | Solution | 252 | | 200 | supplied in | 20 mM |
| | | | | | | | | | Tris- |
| | | | | | | | | solution | HCl, |
| | | | | | | | | | pH 8 |
| | | | | | | | | | with |
| | | | | | | | | | 100 mM |
| | | | | | | | | | NaCl |
| 178 | LGALS7 | lectin, | SRP4647 | Normal | 1000-7000 | | 250 | 1000 | PBS w |
| | | galactoside- | | | | | | | 0.1% |
| | | binding, | | | | | | | BSA |
| | | soluble, 7 | | | | | | | |
| 179 | LIPC | lipase, | L9780 | Units/ | 0.025-0.056 | u/mL | 0.01 | supplied as | PBS |
| | | hepatic | | Solution | | | (0.166 U/ml) | solution | |
| 180 | LTA | lymphotoxin | T7799 | Normal | 125-500 | | 100 | 100 | PBS w |
| | | alpha (TNF | | | | | | | 0.1% |
| | | superfamily, | | | | | | | BSA |
| | | member 1) | | | | | | | |
| 181 | MIA | melanoma | SRP4887 | Normal | 100 | | 100 | 1000 | DDW |
| | | inhibitory | | | | | | | |
| | | activity | | | | | | | |
| 182 | NGF | nerve growth | N1408 | Normal | 0.04-0.2 | | 100 | 100 | PBS w |
| | | factor (beta | | | | | | | 0.1% |
| | | polypeptide) | | | | | | | BSA |
| 183 | NOG | noggin | SRP4675 | Normal | 40-200 | | 100 | 250 | PBS w |
| | | | | | | | | | 0.1% |
| | | | | | | | | | BSA |
| 184 | IL21 | interleukin 21 | SRP3087 | Normal | 4-50 | | 25 | 100 | PBS w |
| | | | | | | | | | 0.1% |
| | | | | | | | | | BSA |
| 185 | NRG1 | neuregulin 1 | 5898-NR-050 | Normal | 20-100 | | 50 | 100 | PBS w |
| | (alpha) | alpha | (R&D) | | | | | | 0.1% |
| | | | | | | | | | BSA |
| 186 | NRTN | neurturin | SRP3124 | Normal | 20-100 | | 80 | 100 | 4 mM |
| | | | | | | | | | HCl w |
| | | | | | | | | | 0.1% |
| | | | | | | | | | BSA |
| 187 | NTF3 | neurotrophin 3 | N1905 | Normal | 1.0-10.0 | | 25 | 50 | PBS w |
| | | | | | | | | | 0.1% |
| | | | | | | | | | BSA |
| 188 | ORM2 | orosomucoid 2 | G9885 | Normal | 2000 | | 2000 | 10000 | DDW |
| 189 | PDGFB | platelet- | P3201 | Normal | 1.5-6 | | 20 | 100 | 4 mM |
| | | derived growth | | | | | | | HCl |
| | | factor beta | | | | | | | |
| | | polypeptide | | | | | | | |
| 190 | PDGFC | platelet | SRP3139 | Normal | 15-350 | | 100 | 100 | 4 mM |
| | | derived growth | | | | | | | HCl w |
| | | factor C | | | | | | | 0.1% |
| | | | | | | | | | BSA |
| 191 | PROK2 | prokineticin 2 | SRP3146 | Normal | 100-400 | | 200 | 100 | PBS |
| 192 | PROZ | protein Z, | P7489 | Solution | plasma: 141 | | 150 | supplied in | PBS |
| | | vitamin K- | | | | | | solution | |
| | | dependent | | | | | | | |
| | | plasma | | | | | | | |
| | | glycoprotein | | | | | | | |
| 193 | RLN1 | relaxin 1 | R2156 | Normal | 8.0-40 | | 50 | 10 | PBS w |
| | | | | | | | | | 0.1% |
| | | | | | | | | | BSA |
| 194 | RLN2 | relaxin 2 | SRP3147 | Normal | 0.5-2.5 | | 100 | 1000 | PBS w |
| | | | | | | | | | 0.1% |
| | | | | | | | | | BSA |
| 195 | SERPINF1 | serpin peptidase | SRP4988 | Normal | 150-750 | | 200 | 250 | 20 mM |
| | | inhibitor, clade | | | | | | | Tris- |
| | | F (alpha-2 | | | | | | | HCl, |
| | | antiplasmin, | | | | | | | pH 8 |
| | | pigment epithelium | | | | | | | |
| | | derived factor), | | | | | | | |
| | | member 1 | | | | | | | |
| 196 | SERPINI1 | serpin peptidase | SRP3123 | Normal | 300-600 | | 250 | 1000 | 20 mM |
| | | inhibitor, clade | | | | | | | Tris- |
| | | I (neuroserpin), | | | | | | | HCl, |
| | | member 1 | | | | | | | pH 8 |
| 197 | SFN | stratifin | S5171 | Solution | 750 | | 1000 | supplied as | PBS |
| | | | | | | | | solution | |
| 198 | SHBG | sex hormone- | S1437 | Solution | Plasma: 8-28 | | 50 | supplied as | PBS |
| | | binding globulin | | | | | | solution | |
| 199 | SPP1 | secreted | SRP3131 | Normal | 10-100 | | 100 | 100 | PBS w |
| | | phosphoprotein 1 | | | | | | | 0.1% |
| | | | | | | | | | BSA |
| 200 | TGFB1 | transforming | T7039 | Normal | 0.04-0.2 | | 10 | 10 | 4 mM |
| | | growth factor, | | | | | | | HCl w |
| | | beta 1 | | | | | | | 0.1% |
| | | | | | | | | | BSA |
| 201 | TGFB2 | transforming | SRP3170 | Normal | 0.025-0.25 | | 10 | 20 | 4 mM |
| | | growth factor, | | | | | | | HCl w |
| | | beta 2 | | | | | | | 0.1% |
| | | | | | | | | | BSA |
| 202 | TGFB3 | transforming | SRP3171 | Normal | 0.01-0.05 | | 10 | 20 | 4 mM |
| | | growth factor, | | | | | | | HCl w |
| | | beta 3 | | | | | | | 0.1% |
| | | | | | | | | | BSA |
| 203 | THBD | thrombomodulin | SRP3172 | Normal | 12.58 | | 10 | 1000 | DDW |
| 204 | TIMP3 | TIMP | T1327 | Normal | 66; 3 nM | | 80 | 100 | DDW |
| | | metallopeptidase | | | | | | | |
| | | inhibitor 3 | | | | | | | |
| 205 | TNF | tumor necrosis | T6674 | Normal | 0.025-0.1 | | 25 | 100 | PBS w |
| | | factor | | | | | | | 0.1% |
| | | | | | | | | | BSA |
| 206 | TNFRSF11A | tumor necrosis | T3573 | Normal | 1.5-7.5 | | 20 | 100 | PBS w |
| | | factor receptor | | | | | | | 0.1% |
| | | superfamily, | | | | | | | BSA |
| | | member 11a, | | | | | | | |
| | | NFKB activator | | | | | | | |
| 207 | TNFRSF11B | tumor necrosis | SRP3132 | Normal | 8-24.0 | | 100 | 10 | PBS w |
| | | factor receptor | | | | | | | 0.1% |
| | | superfamily, | | | | | | | BSA |
| | | member 11b | | | | | | | |
| 208 | TNFRSF17 | tumor necrosis | SRP3010 | Normal | 10-40.0 | | 50 | 1000 | PBS |
| | | factor receptor | | | | | | | |
| | | superfamily, | | | | | | | |
| | | member 17 | | | | | | | |
| 209 | TNFRSF1A | tumor necrosis | SRP3162 | Normal | 45-90 | | 50 | 10 | PBS w |
| | | factor receptor | | | | | | | 0.1% |
| | | superfamily, | | | | | | | BSA |
| | | member 1A | | | | | | | |
| 210 | TNFRSF1B | tumor necrosis | SRP3163 | Normal | 125-600 | | 200 | 1000 | PBS w |
| | | factor receptor | | | | | | | 0.1% |
| | | superfamily, | | | | | | | BSA |
| | | member 1B | | | | | | | |
| 211 | TNFRSF6B | tumor necrosis | D2441 | Normal | 30-150 | | 100 | 100 | PBS |
| | | factor receptor | | | | | | | |
| | | superfamily, | | | | | | | |
| | | member 6b, decoy | | | | | | | |
| 212 | TNFSF11 | tumor necrosis | SRP3161 | Normal | 10-25.0 | | 25 | 100 | PBS w |
| | | factor (ligand) | | | | | | | 0.1% |
| | | superfamily, | | | | | | | BSA |
| | | member 11 | | | | | | | |
| 213 | TNFSF12 | tumor necrosis | SRP4360 | Normal | 2.0-250 | | 25 | 100 | PBS w |
| | | factor (ligand) | | | | | | | 0.1% |
| | | superfamily, | | | | | | | BSA |
| | | member 12 | | | | | | | |
| 214 | TNFSF13 | tumor necrosis | SRP3008 | Normal | 5-25 | | 25 | 100 | PBS w |
| | | factor (ligand) | | | | | | | 0.1% |
| | | superfamily, | | | | | | | BSA |
| | | member 13 | | | | | | | |
| 215 | TNFSF13B | tumor necrosis | B6681 | Normal | 0.4-2 | | 20 | 100 | PBS w |
| | | factor (ligand) | | | | | | | 0.1% |
| | | superfamily, | | | | | | | BSA |
| | | member 13b | | | | | | | |
| 216 | TNFSF14 | tumor necrosis | SRP3106 | Normal | 1-4 | | 20 | 1000 | PBS w |
| | | factor (ligand) | | | | | | | 0.1% |
| | | superfamily, | | | | | | | BSA |
| | | member 14 | | | | | | | |
| 217 | TPO | thyroid | SRP3178 | Normal | 0.3-3 | | 50 | 1000 | PBS w |
| | | peroxidase | | | | | | | 0.1% |
| | | | | | | | | | BSA |
| 218 | UMOD | uromodulin | T2702 | Normal | Serum: 241 | | 250 | 80000 | DDW |
| 219 | VEGFA | vascular | SRP3029 | Normal | 100-400 | | 200 | 1000 | PBS w |
| | | endothelial | | | | | | | 0.1% |
| | | growth factor A | | | | | | | BSA |
| 220 | VEGFB | vascular | SRP3183 | Normal | 10-2000 | | 200 | 1000 | PBS w |
| | | endothelial | | | | | | | 0.1% |
| | | growth factor B | | | | | | | BSA |
| 221 | VEGFC | vascular | SRP3184 | Normal | 200-800 | | 200 | 1000 | PBS w |
| | | endothelial | | | | | | | 0.1% |
| | | growth factor C | | | | | | | BSA |
| 222 | WISP3 | WNT1 inducible | SRP3188 | Normal | 200-300 | | 200 | 1000 | 10 mM |
| | | signaling pathway | | | | | | | Acetic |
| | | protein 3 | | | | | | | Acid w |
| | | | | | | | | | 0.1% |
| | | | | | | | | | BSA |
| 223 | XCL1 | chemokine (C | L9788 | Normal | 50 | | 50 | 100 | PBS w |
| | | motif) ligand 1 | | | | | | | 0.1% |
| | | | | | | | | | BSA |
| 224 | ADM | adrenomedullin | A2327 | Normal | Plasma: 1-3 | | 20 | 1000 | 10mM |
| | | | | | | | | | Acetic |
| | | | | | | | | | Acid |
| 225 | AREG | amphiregulin | A7080 | Normal | 5-15.0 | | 30 | >10 | PBS w |
| | | | | | | | | | 0.1% |
| | | | | | | | | | BSA |
| 226 | CARTPT | CART prep | C5977 | Normal | plasma: 0.53-1.19 | | 10 | 50000-100000 | DDW |
| | | ropeptide | | | | | | | |
| 227 | CCL11 | chemokine (C-C | SRP4028 | Normal | 1-5 | | 15 | 1000 | PBS w |
| | | motif) ligand 11 | | | | | | | 0.1% |
| | | | | | | | | | BSA |
| 228 | CCL17 | chemokine (C-C | SRP4333 | Normal | 1-10.0 | | 15 | 1000 | PBS w |
| | | motif) ligand 17 | | | | | | | 0.1% |
| | | | | | | | | | BSA |
| 229 | CCL19 | chemokine (C-C | M3552 | Normal | 5.3 | | 15 | >25 | PBS w |
| | | motif) ligand 19 | | | | | | | 0.1% |
| | | | | | | | | | BSA |
| 230 | CCL22 | chemokine (C-C | M251 | Normal | 2-6 | | 15 | >25 | PBS w |
| | | motif) ligand 22 | | | | | | | 0.1% |
| | | | | | | | | | BSA |
| 231 | CCL3L1 | chemokine (C-C | SRP3104 | Normal | 1.0-10.0 | | 15 | 1000 | PBS w |
| | | motif) ligand 3- | | | | | | | 0.1% |
| | | like 1 | | | | | | | BSA |
| 232 | CCL4L1 | chemokine (C-C | SRP3103 | Normal | 0.1-10 | | 15 | 1000 | PBS w |
| | | motif) ligand 4- | | | | | | | 0.1% |
| | | like 1 | | | | | | | BSA |
| 233 | CHGA | chromogranin A | C6249 | Normal | plasma: 41.6-204 | | 100 | 2000 | DDW |
| | | (parathyroid | | | | | | | |
| | | secretory | | | | | | | |
| | | protein 1) | | | | | | | |
| 234 | COL4A6 | collagen, type | C5533 | Normal | 1000 | | 400 | 1000 | PBS w |
| | | IV, alpha 6 | | | | | | | 0.1% |
| | | | | | | | | | BSA |
| 235 | CPB1 | carboxypeptidase | P0059 | Solution | Plasma: 10.4 | | 20 | supplied as | PBS |
| | | B1 (tissue) | | | | | | solution | |
| 236 | CRH | corticotropin | C3042 | Normal | plasma: 0.37-0.41 | | 50 | 1000 | DDW |
| | | releasing | | | | | | | |
| | | hormone | | | | | | | |
| 237 | CSF2 | colony | SRP3050 | Normal | 0.006-0.1 | | 2 | 100 | PBS w |
| | | stimulating | | | | | | | 0.1% |
| | | factor 2 | | | | | | | BSA |
| | | (granulocyte- | | | | | | | |
| | | macrophage) | | | | | | | |
| 238 | CXCL11 | chemokine (C-X-C | I5528 | Normal | 10-20 | | 20 | 100 | PBS w |
| | | motif) ligand 11 | | | | | | | 0.1% |
| | | | | | | | | | BSA |
| 239 | CXCL13 | chemokine (C-X-C | B2929 | Normal | 20 | | 20 | 100 | PBS w |
| | | motif) ligand 13 | | | | | | | 0.1% |
| | | | | | | | | | BSA |
| 240 | CXCL14 | chemokine (C-X-C | SRP3019 | Normal | 1.0-10.0 | | 15 | 1000 | PBS w |
| | | motif) ligand 14 | | | | | | | 0.1% |
| | | | | | | | | | BSA |
| 241 | EDN1 | endothelin 1 | E7764 | Normal | 0.05 | | 1 | 100 | DDW |
| 242 | EDN2 | endothelin 2 | E9012 | Normal | 0.001 | | 1 | 100 | PBS |
| 243 | EDN3 | endothelin 3 | E9137 | Normal | 0.05 | | 1 | 100 | DDW |
| 244 | EFNA3 | ephrin-A3 | E0278 | Normal | 0.31-20 | | 40 | >100 | PBS |
| 245 | EFNA4 | ephrin-A4 | E0403 | Normal | 0.16-10 | | 25 | >100 | PBS |
| 246 | EFNA5 | ephrin-A5 | E0528 | Normal | 0.078-5 | | 25 | >100 | PBS |
| 247 | F13A1 | coagulation | F0166 | Normal | Plasma: 30 | | 50 | 10000 | DDW |
| | | factor XIII, A1 | | | | | | | |
| | | polypeptide | | | | | | | |
| 248 | FGF16 | fibroblast | SRP3038 | Normal | 0.5-30 | | 20 | 100 | PBS w |
| | | growth factor 16 | | | | | | | 0.1% |
| | | | | | | | | | BSA |
| 249 | FGF18 | fibroblast | SRP4082 | Normal | 0.5 | | 20 | 1000 | 20 mM |
| | | growth factor 18 | | | | | | | Tris- |
| | | | | | | | | | HCl, |
| | | | | | | | | | pH 8 |
| 250 | FGF2 | fibroblast | SRP4037 | Normal | 7-70.0 | | 25 | 1000 | 20 mM |
| | | growth factor 2 | | | | | | | Tris- |
| | | (basic) | | | | | | | HCl, |
| | | | | | | | | | pH 8 |
| 251 | FGF4 | fibroblast | F8424 | Normal | 0.5 | | 20 | 1000 | DDW |
| | | growth factor 4 | | | | | | | |
| 252 | FGF5 | fibroblast | F4537 | Normal | 2-10 | | 20 | 10 | PBS w |
| | | growth factor 5 | | | | | | | 0.1% |
| | | | | | | | | | BSA |
| 253 | FGF8 | fibroblast | SRP4053 | Normal | 0.5 | | 20 | 100 | 20 mM |
| | | growth factor 8 | | | | | | | Tris- |
| | | (androgen- | | | | | | | HCl, |
| | | induced) | | | | | | | pH 8 |
| 254 | FGFR1 | fibroblast | F9174 | Normal | 1-3 | | 10 | 100 | PBS w |
| | | growth factor | | | | | | | 0.1% |
| | | receptor 1 | | | | | | | BSA |
| 255 | FLT3 | fms-related | F7426 | Normal | 10-30 | | 30 | 1000 | PBS w |
| | | tyrosine kinase 3 | | | | | | | 0.1% |
| | | | | | | | | | BSA |
| 256 | FOLR2 | folate receptor | F7057 | Normal | 0.2-1 | | 10 | 100 | PBS |
| | | 2 (fetal) | | | | | | | |
| 257 | GAL | galanin | G0278 | Normal | plasma: 13.46-21.51 | | 15 | 1000 | DDW |
| | | prepropeptide | | | | | | | |
| 258 | GDF2 | growth | SRP3049 | Normal | 0.5-1.9 | | 5 | 10 | DDW |
| | | differentiation | | | | | | | |
| | | factor 2 | | | | | | | |
| 259 | GH1 | growth hormone 1 | S4776 | Normal | 0.097 | | 10 | >10 | PBS w |
| | | | | | | | | | 0.1% |
| | | | | | | | | | BSA |
| 260 | GHRH | growth hormone | G3644 | Normal | 35 | | 50 | 1000 | DDW |
| | | releasing | | | | | | | |
| | | hormone | | | | | | | |
| 261 | GIP | gastric | G2269 | Normal | 0.81 | | 25 | 1000 | DDW |
| | | inhibitory | | | | | | | |
| | | polypeptide | | | | | | | |
| 262 | GRP | gastrin- | G8022 | Normal | Plasma: 0.05 | | 10 | 2000 | DDW |
| | | releasing | | | | | | | |
| | | peptide | | | | | | | |
| 263 | HBEGF | heparin- | SRP3052 | Normal | 0.15-1 | | 25 | 250 | PBS w |
| | | binding | | | | | | | 0.1% |
| | | EGF-like | | | | | | | BSA |
| | | growth factor | | | | | | | |
| 264 | IFNG | interferon, | SRP3058 | Normal | 5.0-10.0 | | 10 | 200 | DDW |
| | | gamma | | | | | | | |
| 265 | IGF2 | insulin-like | SRP3070 | Normal | 2 | | 20 | 200 | PBS |
| | | growth factor 2 | | | | | | | |
| | | (somatomedin A) | | | | | | | |
| 266 | IGFBP7 | insulin-like | SRP3066 | Normal | 4-20 | | 20 | 100 | PBS |
| | | growth factor | | | | | | | |
| | | binding | | | | | | | |
| | | protein 7 | | | | | | | |
| 267 | IL17 | interleukin 17 | SRP3080 | Normal | 0.25-1.25 | | 20 | 1000 | DDW |
| 268 | IL17D | interleukin 17D | SRP3082 | Normal | 10 | | 20 | 1000 | DDW |
| 269 | IL17E | interleukin 17E | SRP4176E | Normal | 10 | | 20 | 10 | 4 mM |
| | | | | | | | | | HCl w |
| | | | | | | | | | 0.1% |
| | | | | | | | | | BSA |
| 270 | IL17F | interleukin 17F | SRP4176F | Normal | 10 | | 20 | 10 | 4 mM |
| | | | | | | | | | HCl w |
| | | | | | | | | | 0.1% |
| | | | | | | | | | BSA |
| 271 | IL1B | interleukin 1, | SRP3083 | Normal | 0.001-0.012 | | 5 | 25 | PBS w |
| | | beta | | | | | | | 0.1% |
| | | | | | | | | | BSA |
| 272 | IL3 | interleukin | SRP4134 | Normal | 0.02-0.1 | | 5 | 100 | PBS w |
| | | 3 (colony- | | | | | | | 0.1% |
| | | stimulating | | | | | | | BSA |
| | | factor, multiple) | | | | | | | |
| 273 | LCN2 | lipocalin 2 | SRP4928 | Normal | plasma: 43.8-82.6 | | 40 | 1000 | DDW |
| 274 | LEP | leptin | L4146 | Normal | 0.4-2 | | 100 | 1000 | 20 mM |
| | | | | | | | | | Tris- |
| | | | | | | | | | HCl, |
| | | | | | | | | | pH 8 |
| | | | | | | | | | with |
| | | | | | | | | | 100 mM |
| | | | | | | | | | NaCl |
| 275 | LIF | leukemia | L5283 | Solution | 0.3 | | 1.65 | 5 | PBS w |
| | | inhibitory factor | | | | | | | 0.1% |
| | | | | | | | | | BSA |
| 276 | LYZ | lysozyme | L1667 | Normal | 4000-13000 | | 5000 | 10000 | PBS |
| | | | | | | | | (50 mg/ml | |
| | | | | | | | | reported) | |
| 277 | MMP12 | matrix | M9695 | Solution | ~1 | | 5 | supplied in | PBS |
| | | metallopeptidase | | | | | | solution | |
| | | 12 (macrophage | | | | | | | |
| | | elastase) | | | | | | | |
| 278 | NRG1 | neuregulin 1 | 377-HB-050 | Normal | 2.5-12.5 | | 15 | 100 | PBS w |
| | (beta) | beta | (R&D) | | | | | | 0.1% |
| | | | | | | | | | BSA |
| 279 | NTS | neurotensin | N6383 | Normal | 170 | | 200 | 20000 | 10 mM |
| | | | | | | | | | acetic |
| | | | | | | | | | acid |
| 280 | PF4 | platelet | SRP3142 | Normal | 1-10.0 | | 15 | 100 | PBS w |
| | | factor 4 | | | | | | | 0.1% |
| | | | | | | | | | BSA |
| 281 | PGF | placental | P1588 | Normal | 0.1-5 | | 5 | >10 | PBS w |
| | | growth factor | | | | | | | 0.1% |
| | | | | | | | | | BSA |
| 282 | PPY | pancreatic | P9903 | Normal | plasma: 0.142-1.564 | | 10 | 1000 | PBS |
| | | polypeptide | | | | | | | |
| 283 | PRL | prolactin | L4021 | Normal | 0.25-1 | | 10 | 100 | PBS w |
| | | | | | | | | | 0.1% |
| | | | | | | | | | BSA |
| 284 | PRSS1 | protease, serine, | T6424 | Normal | 86 | | 50 | | DDW |
| | | 1 (trypsin 1) | | | | | | | |
| 285 | PSPN | persephin | SRP3141 | Normal | 0.1-16 | | 16 | 100 | 4 mM |
| | | | | | | | | | HCl w |
| | | | | | | | | | 0.1% |
| | | | | | | | | | BSA |
| 286 | PTHLH | parathyroid | SRP4651 | Normal | 50 | | 40 | 1000 | DDW |
| | | hormone-like | | | | | | | |
| | | hormone | | | | | | | |
| 287 | PYY | peptide YY | P1306 | Normal | plasma: 43.9-80.9 | | 50 | 1000 | DDW |
| 288 | RETNLB | resistin like | SRP4654 | Normal | 20 | | 20 | 1000 | DDW |
| | | beta | | | | | | | |
| 289 | RLN3 | relaxin 3 | R2031 | Normal | 3.5-17.5 | | 20 | 100 | PBS w |
| | | | | | | | | | 0.1% |
| | | | | | | | | | BSA |
| 290 | SERPINA1 | serpin peptidase | A9024 | Normal | 200, 5 nM | | 250 | 1000 | 20 mM |
| | | inhibitor, clade | | | | | | | Tris- |
| | | A (alpha-1 | | | | | | | HCl, |
| | | antiproteinase, | | | | | | | pH 8 |
| | | antitrypsin), | | | | | | | |
| | | member 1 | | | | | | | |
| 291 | SERPINC1 | serpin peptidase | A2221 | Normal | 5 | | 20 | 100 | 20 mM |
| | | inhibitor, clade | | | | | | | Tris- |
| | | C (antithrombin), | | | | | | | HCl, |
| | | member 1 | | | | | | | pH 8 |
| 292 | SOD3 | superoxide | S9636 | Units | 0.1 | units/ml | 0.1 | 80000 U/ml | DDW |
| | | dismutase 3, | | | | | (1.66 U/ml) | | |
| | | extracellular | | | | | | | |
| 293 | SST | somatostatin | S1763 | Normal | 100 | | 10 | 1000 | DDW |
| 294 | TGFA | transforming | T7924 | Normal | 0.1-0.4 | | 10 | 1000 | DDW |
| | | growth factor, | | | | | | | |
| | | alpha | | | | | | | |
| 295 | TGFBR3 | transforming | T4567 | Normal | 10-50.0 | | 50 | 200 | PBS w |
| | | growth factor, | | | | | | | 0.1% |
| | | beta receptor III | | | | | | | BSA |
| 296 | VIP | vasoactive | V3628 | Normal | 2-200 | | 150 | 1000 | DDW |
| | | intestinal | | | | | | | |
| | | peptide | | | | | | | |
| 297 | WISP2 | WNT1 inducible | SRP3022 | Normal | 10-20 | | 15 | 100 | DDW |
| | | signaling | | | | | | | |
| | | pathway protein 2 | | | | | | | |
| 298 | FASLG | Fas ligand | SRP3036 | Normal | 1.5 | | 7 | 1000 | PBS w |
| | | (TNF superfamily, | | | | | | | 0.1% |
| | | member 6) | | | | | | | BSA |
| 299 | BMP3 | bone | SRP4573 | Normal | 100 | | 75 | 1000 | PBS w |
| | | morphogenetic | | | | | | | 0.1% |
| | | protein 3 | | | | | | | BSA |
| 300 | CTGF | connective | SRP4702 | Normal | 50-100 | | 100 | 1000 | DDW |
| | | tirruse | | | | | | | |
| | | growth factor | | | | | | | |
| 301 | CYR61 | cysteine rich | SRP3024 | Normal | 10-100 | | 100 | 1000 | PBS w |
| | | angiogenci | | | | | | | 0.1% |
| | | inducer 61 | | | | | | | BSA |
| 302 | GDF15 | growth | G3046 | Normal | | | 250 | | PBS w |
| | | differentiation | | | | | | | 0.1% |
| | | factor 15 | | | | | | | BSA |
| 303 | LAMA1 | laminin | L2020 | Solution | 5000 | | 5000 | supplied as | DDW |
| | | subunit alpha1 | | | | | | solution | |
| 304 | GYPA | glycophorin A | G5017 | Normal | 20 | | 100 | | DDW |
| 305 | LALBA | lactoalbumin | L7269 | Normal | 70-1000 | | 1000 | 3000 | DDW |
| | | alpha | | | | | | | |
| 306 | LTF | lactotransferrin | L4040 | Normal | 1-1000 | | 1000 | 2000 | PBS |
| 307 | MDK | midkine | M3441 | Normal | | | 100 | | PBS |
| 308 | NOV | nephroblastoma | SRP3125 | Normal | | | 200 | 250 | PBS |
| | | overexpressed | | | | | | | |
| 309 | NPPB | natriuretic | B5900 | Normal | ~1 | ng/ml | 10 | 100 | DDW |
| | | peptide B | | | | | | | |
| 310 | OTOR | otoraplin | SRP4987 | Normal | 10 | | 20 | 1000 | DDW |
| 311 | PLA2G7 | phospholipase A2 | SRP3136 | Normal | 5 | | 20 | 1000 | PBS w |
| | | group VII | | | | | | | 0.1% |
| | | | | | | | | | BSA |
| 312 | PLAU | plasminogen | U0633 | Normal | 1.5-21 | | 20 | | DDW |
| | | activator, | | | | | | | |
| | | urokinase | | | | | | | |
| 313 | PLG | plasminogen | P1867 | Normal | High | | 1500 | | DDW |
| 314 | TFF1 | trefoil factor 1 | SRP4893 | Normal | 100-400 | | 150 | 1000 | DDW |
| 315 | TFF2 | trefoil factor 2 | SRP4745 | Normal | 40 | | 150 | 1000 | DDW |
| 316 | TFF3 | trefoil factor 3 | SRP3169 | Normal | 20-100 | | 150 | 1000 | DDW |
| 317 | WISP1 | WNT1 inducible | SRP3187 | Normal | | | 200 | | PBS w |
| | | signaling | | | | | | | 0.1% |
| | | pathway protein 1 | | | | | | | BSA |
| 318 | CGA | glycoprotein | C6322 | Normal | 1-50 | | 50 | 100 | PBS |
| | | hormones | | | | | | | |
| | | alpha polypeptide | | | | | | | |
| 319 | DEFB 1 | defensin beta 1 | D9565 | Normal | 37 | | 50 | 1000 | 10 mM |
| | | | | | | | | | Acetic |
| | | | | | | | | | acid |
| 320 | HPX | hemopexin | H9291 | Normal | | | 1000 | | DDW |
| 321 | QSOX1 | quiescin Q6 | QSOX1 | solution | 50 | nM | 50 nM | | |
| | | sulfhydryl | | | | | | | |
| | | oxidase 1 | | | | | | | |
| 322 | CSF1 | colony | M6518 | Normal | 0.5-5 | | 100 | 500 | PBS w |
| | | stimulating | | | | | | | 0.1% |
| | | factor 1 | | | | | | | BSA |
| | | (macrophage) | | | | | | | |
| 323 | HGF | hepatocyte | H9661 | Normal | 20-40 | | 50 | 100 | PBS w |
| | | growth factor | | | | | | | 0.1% |
| | | (hepapoietin A; | | | | | | | BSA |
| | | scatter factor) | | | | | | | |
| 324 | CCL14 | chemokine (C-C | SRP3054 | Normal | 200-15000 | | 100 | 100-1000 | DDW |
| | | motif) ligand 14 | | | | | | | |
| 325 | CCL3 | chemokine (C-C | SRP4244 | Normal | 2-11 | | 15 | 100-1000 | DDW |
| | | motif) ligand 3 | | | | | | | |
| 326 | CCL4 | chemokine (C-C | SRP3115 | Normal | 3.0-30.0 | | 15 | 100 | PBS w |
| | | motif) ligand 4 | | | | | | | 0.1% |
| | | | | | | | | | BSA |
| 327 | CX3CL1 | chemokine (C-X3-C | F135 | Normal | 0.3-100 | | 50 | >25 | PBS w |
| | | motif) ligand 1 | | | | | | | 0.1% |
| | | | | | | | | | BSA |
| 328 | IL | interleukin 9 | SRP3099 | Normal | 0.1-0.6 | | 25 | 1000 | PBS w |
| | | | | | | | | | 0.1% |
| | | | | | | | | | BSA |
| 329 | LIPC | lipase, hepatic | BCR693 | Units/ | 0.025-0.056 | u/mL | 0.01 | 1 ml | DDW |

| | | | | | | | | |
|---|---|---|---|---|---|---|---|---|
| | | | | Solution | | (0.166 U/ml) | lyophilazed | |
| 330 | CCL19 | chemokine (C-C | SPR4494 | Normal | 5.3 | 15 | 100-1000 | DDW |
| | | motif) ligand 19 | | | | | | |
| 331 | CCL22 | chemokine (C-C | SRP3111 | Normal | 2-6 | 15 | 100-1000 | PBS w |
| | | motif) ligand 22 | | | | | | 0.1% |
| | | | | | | | | BSA |
| 332 | FGF18 | fibroblast | F7301 | Normal | 0.5 | 20 | 1000 | 20 mM |
| | | growth factor 18 | | | | | | Tris- |
| | | | | | | | | HCl, |
| | | | | | | | | pH 8 |
| 333 | VEGFA | vascular | V7259 | Normal | 1-10 | 20 | | PBS w |
| | | endothelial | | | | | | 0.1% |
| | | growth factor A | | | | | | BSA |

Human Biopsies—Human tissue samples were taken in accordance with the Institutional Regulation Board of the Weizmann Institute and after receiving Helsinki Medical Ethics Committee approval from the hospitals that participated in the study. All patients signed informed consent to both take the tissues and to perform DNA sequencing on these tissues in addition to ex vivo culture. Tumor tissue was obtained from patients at the time of operation or at the time of tissue core biopsy. Following resection, the fresh tissue was placed in ice-cold PBS for immediate transfer to the lab for ex vivo organ culturing. Specimens were coded anonymously prior to their arrival to the lab.

High-throughput secretome screens—To screen for the effect of secreted factors on the response of cell lines to different drugs, the following seven-days procedure was performed. On day 0, GFP expressing cells were seeded at 1500-2000 cells per well on 384-wells plates (Corning, 3712), depending on the cell line's proliferation rate. Each plate was seeded with one cell line. At day 1, each plate was treated with the secretome library (Table 2 hereinabove), one well per factor. Immediately afterwards, each plate was treated either with a drug (Table 1 hereinabove) at the EC90 concentration or with DMSO control. The CyBi liquid handler was used to treat each plate with a drug and the secretome library as well as to replace the medium, drug and secreted factors following 3 days of incubation (day 4). Cell fluorescence was read at days 1, 4 and 7 by Cytation3, and for some of the experiments at day 6 as well. Wells of interest were imaged at day 7 using the Operetta (PerkinElmer).

Secretome screens meta-analysis—To select for secreted factors exhibiting a significant effect on resistance to a given drug, the following analysis was performed with Matlab scripts:
1—Normalization and noisefiltration: The following normalizations steps were performed to minimize biases: (1) When possible, plate fluorescence was read in two opposite orientations and values were averaged to avoid instrument-reading biases. (2) Based on observations, the plate area spanning rows 4-13 and columns 4-21 was hardly affected by plate margins biases. This area is referred to herein as plate core. To control for row specific and column specific biases, plate outliers (Zscore>4 or Zscore<−4) and edges were masked. Next, to capture row and column (col) biases for the i-th row and the j-th column, the bias factor was calculated:

$${N}_{i} = \frac{mean ( {r}_{i} )}{mean ( plate core )} ; \qquad {N}_{j} = \frac{mean ( {c}_{j} )}{mean ( plate core )}$$

Finally, each well value (including the masked outliers wells) was divided by the product of row and col bias factors:

$$norm {well}_{i , j} = \frac{{well}_{i , j}}{( {N}_{i} * {N}_{j} )}$$

(3) To control for secreted factor specific auto-fluorescence biases, a background plate that contained the secretome library without cells was incubated for 7 days and fluorescence values were read at days 1, 4, 6, 7. For each plate, for each time point, background values were subtracted. Finally, for each factor, day 1 fluorescence was subtracted from the later time points.
2—Optimal end point selection—Cell fluorescence may reach saturation or even decrease at day 7 due to a too high confluency of the cells in the plate. To address this confluence bias, whenever a day 6 time point was available, the maximum over day 6 and day 7 was selected as the experiment end point. (hereinafter: last_{GFP})
3—Quality control—Plates with suspicious spatial patterns that could arise from technical failures were discarded. Also, plates in which the cells did not grow properly under DMSO or when drug effect on proliferation was less than 30% were discarded as well.

$$cell growth = \frac{{last}_{G F P}}{day {1}_{G F P}} < 1.4$$

$$residual drug growth = \frac{{last}_{G F P} drug}{{last}_{G F P} DMSO} > 0.7$$

Upon QC completion, 199 experiment plates and 79 control (DMSO) plates were left for further analysis.
4—Scoring methods-A GFP value for wells without secretome factors was obtained by averaging over the plate internal control wells. Those wells contained the solvents used in the secretome library.
   pScore: The effect of the secreted factors on proliferation under DMSO was evaluated using pScore (proliferation score). Values were given in percent units.

$$p Score = ( \frac{{last}_{G F P} with factor}{{last}_{G F P} without factor} - 1 ) * 100$$

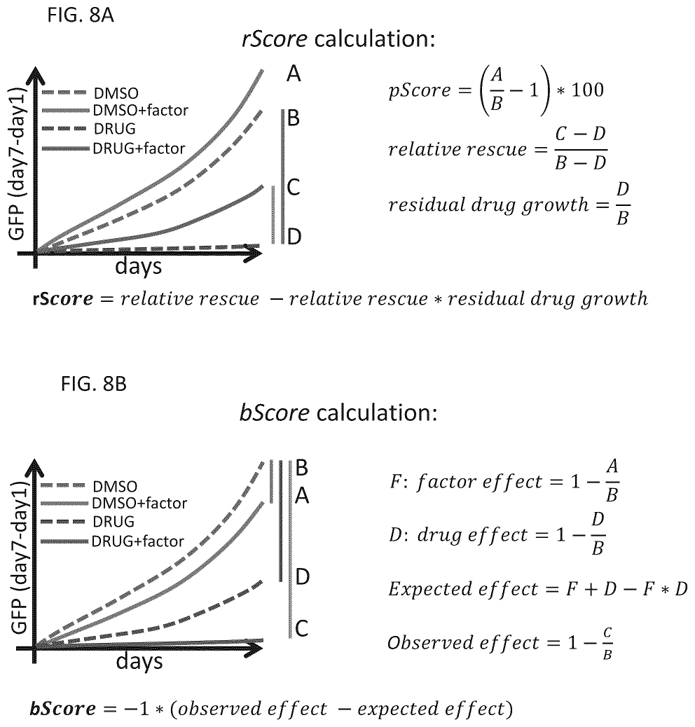

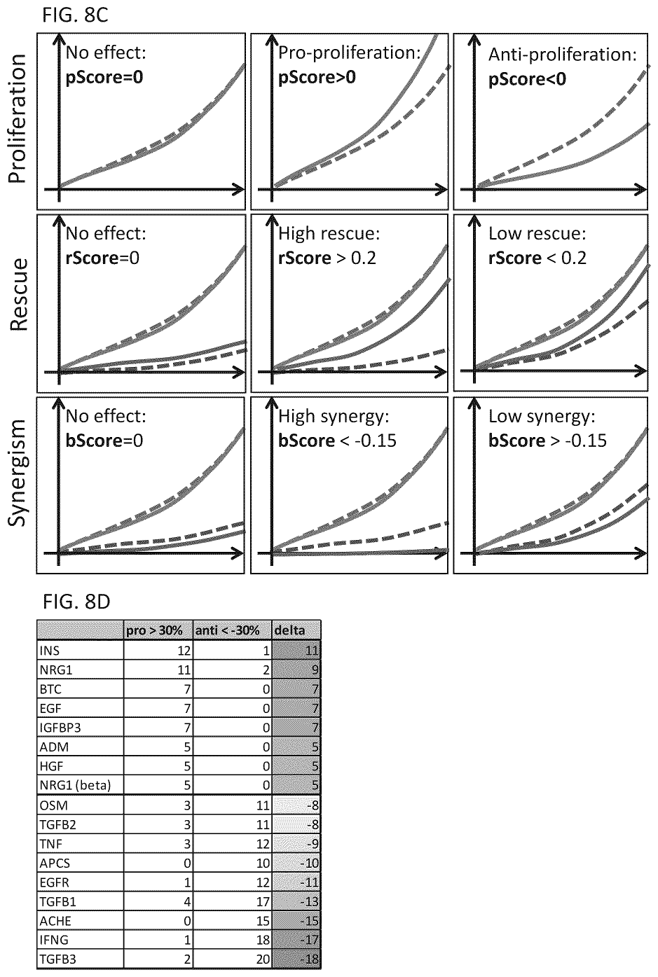

Thus, a positive pScore reflects a pro-proliferative effect of the secreted factor on the cells, while a negative pScore represents an anti-proliferative effect (FIGS. 8A-D).

rScore: The effect of the secreted factors on resistance to anti-cancer drugs was evaluated using rScore (rescue score). rScore was assigned to a given factor under two conditions:
1. drug effect was >30% (residual drug growth<0.7)
2. proliferation ratio between factor treated and untreated cells in the presence of drug was higher than one:

$$proliferation ratio = \frac{{last}_{G F P} drug \& factor}{{last}_{G F P} drug only} > 1$$

First, the ratio reflecting the effect on drug resistance was calculated:

$$S = \frac{{last}_{GFP} drug \& factor - {last}_{GFP} drug only}{las {t}_{GFP} DMSO only - {last}_{GFP} drug only}$$

This ratio was further normalized to also consider the efficacy of the drug and avoid the bias of high values when drug efficacy is small.

rSscore=S−S*residual drug growth

Based on manual inspection of the rScore distribution across the data, the threshold for a potential effect on drug resistance was set to 0.2 (FIGS. 8A-D).

bScore: The synergistic effect of a secreted factor with a drug was evaluated using bScore (bliss score (25)). bScore was assigned to a given factor only when its proliferation ratio was below 1.
drug effect:

$$d = 1 - \frac{{last}_{GFP} drug only}{{last}_{GFP} DMSO only}$$

Secreted factor effect:

$$f = 1 - \frac{{last}_{GFP} factor only}{{last}_{GFP} DMSO only}$$

Observed effect^{(*)}:

$$O = 1 - \frac{{last}_{GFP} drug \& factor}{{last}_{GFP} DMSO only}$$

Expected effect:

E=d+f−d*f

bScore=−1*(0−E)

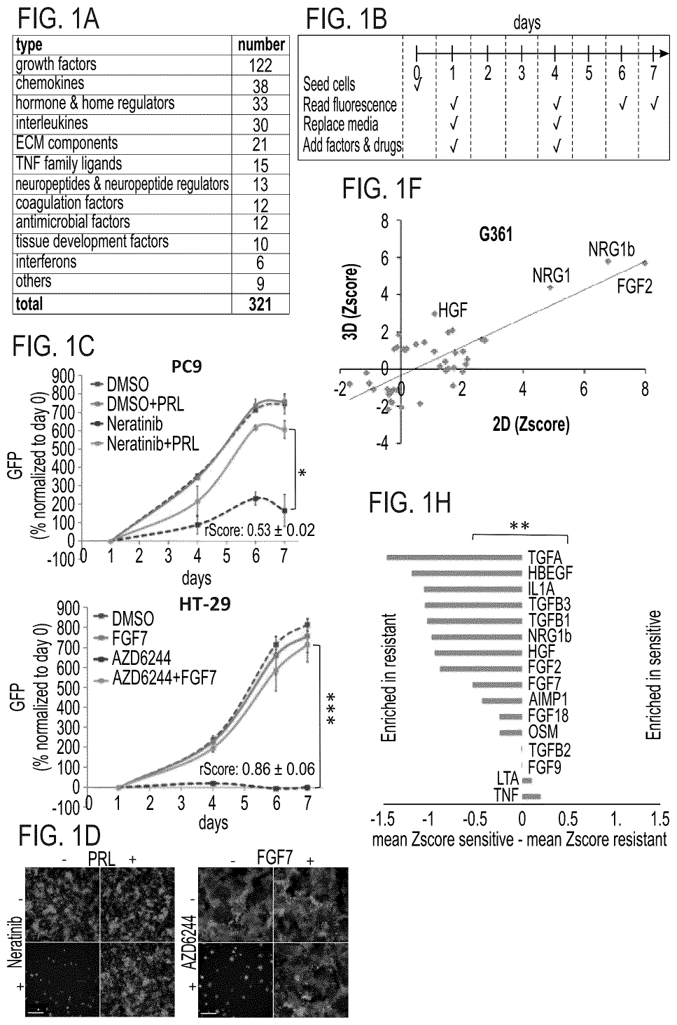

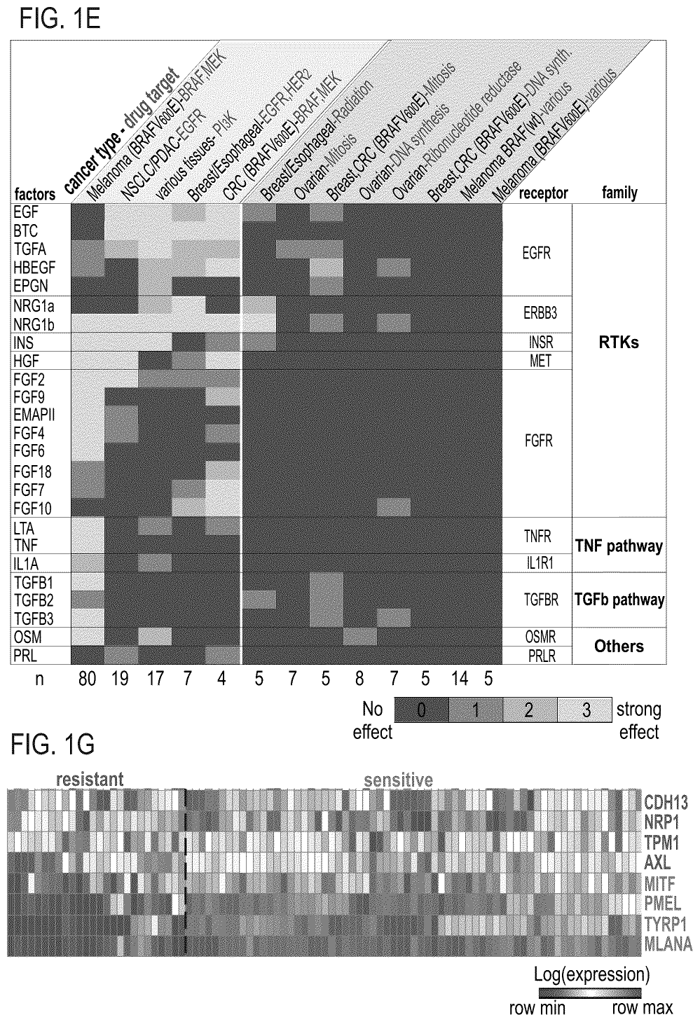

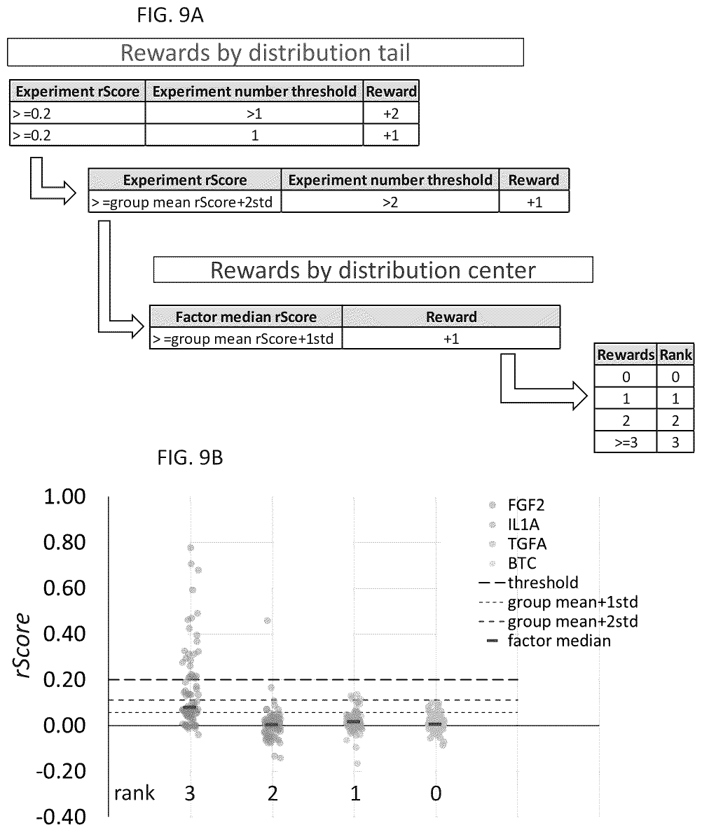

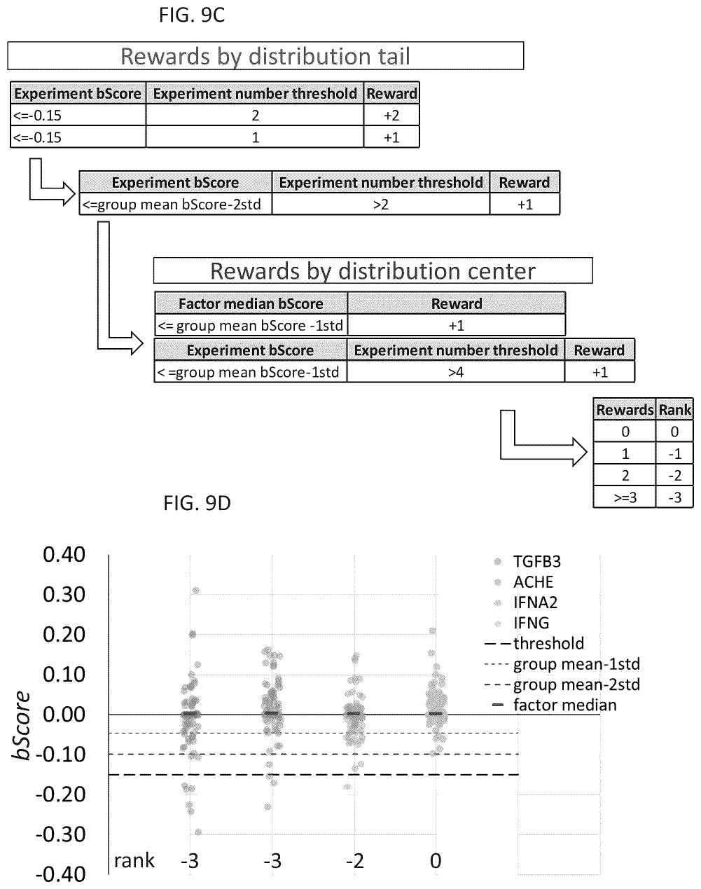

(*) Negative values were considered as zero.
Based on manual inspection of bScore distribution across the data, the threshold for a synergistic effect was set to −0.15 (FIGS. 8A-D).
5—selection of proliferation independent effect on drug response—To filter the more trivial cases where the secreted factor effect on resistance to drug is a mere reflection of the factor's effect on proliferation, at least one instance where proliferation and the effect on drug were impaired was necessary in order for this factor to be considered as hit in FIG. 1E. Thus, a factor with a potential effect on drug resistance was considered when there was at least one case of rScore value>0.2 and proliferation ratio in drug treated cells was at least 2.5 fold higher than the effect of the factor on the proliferation of DMSO treated cells. A factor with a potential synergistic effect was considered when there was at least one case of bScore<−0.15 and proliferation ratio in drug treated cells was at least 2.5 fold lower than the effect of the factor on the proliferation of the DMSO treated cells.
6—ranking the factors by the effect on drug response within a group of experiments—Experiments were grouped based on cancer type, drug target and the similarity between screens' vectors of rScore values resulting in 21 groups of experiments. Groups with less than 4 experiments were discarded, finally resulting in 13 groups of experiments (FIG. 1E). Scores were collapsed to ranks 0, 1, 2, 3 for factors mediating drug resistance and ranks 0, −1, −2, −3 for factors mediating drug synergism as explained in detail by FIGS. 9A-D.

shRNA screen—Screen protocol: To screen for AIMP1 receptors that mediate AIMP1 effect on resistance of melanoma cell lines to BRAF inhibition, two libraries of lenti-viruses, each in 96-wells plate, were prepared by The RNAi Consortium (TRC) at the Broad institute. Briefly, a library of shRNA oligos for AIMP1 receptors and a library of shRNA oligos for FGF receptors (data not shown) were cloned into plasmids with puromycin resistance cassette (pLKO.1, Addgene, 10878). Each library included negative control wells (GFP, Luciferase, lacZ and RFP) and virus-free wells. GFP expressing, melanoma BRAF (V600E) mutated cells were seeded at a concentration of 10⁵ cells/ml, in clear black bottom 96-wellS plates, 5 plates per library. To infect the cells with the library of lenti-viruses, 24 hours following seeding, cells were treated with polybrene (2 μg/ml) and 20 μl of virus per well, then centrifuged at 2000 rpm for 30 μminutes. Virus was washed 24 hours later. To test the infection efficacy, one of the five plates was treated with 0.5 μg/ml puromycin. Clones were expanded for 48 hours, then GFP was read. Per library, each of the remaining four cells plate was treated with either DMSO, BRAF inhibitor (2 μM PLX4720), 50 ng/ml AIMP1 (Novus, NBP1-50936), or the combination of PLX4720+AIMP1. GFP was read again 4, 6 and 7 days post treatment. Prior to GFP reading on day 4, plates were re-treated with fresh reagents.

Quantifying shRNA effect on AIMP1 μmediated resistance to PLX4720: To find receptors whose knock down abrogated the effect of AIMP1 on resistance to PLX4720, rScore abrogation was calculated in the following steps:
1. Finding day of maximal growth: average GFP over virus-free wells in the DMSO treated plate was calculated for day 1, 4, 6, 7 post treatment. Day 6 was found to be the maximal growth time point for both libraries, thus:

last_{GFP}=max(Day6_{GFP}−Day1_{GFP})
Negative values were floored to zero.
2. Calculating residual drug growth and rScore of AIMP1: values to assign for the rScore formula were derived from averaging over virus-free wells in DMSO, PLX420 or PLX4720+ATMP1 treated plates.
3. Efficacy of infection was deduced from growth under puromycin (which is the selection cassette of the infected plasmid), relative to no selection (DMSO). Wells with efficacy of infection lower than 95% were discarded. For well (i,j) efficacy was calculated using the DMSO and puromycin treated plates, as follows:

$${efficacy}_{i , j} = \frac{{last}_{G F P} puromycin}{{last}_{G F P} DMSO}$$

4. Toxicity of infection was deduced from the pScore values following infection. Wells with toxicity higher than 0.5 were discarded: for well (i,j) toxicity was calculated using the DMSO plate, as follows:

toxicity_{i,j}=abs(min(pScore(DMSO plate),0))
5. Calculating rScore for each well in the PLX4720+AIMP1 treated plate: all values but last_{GFP} drug&factor last_{GFP}drug&factorwere derived from averaging over virus-free wells in DMSO and PLX420 treated plates. Following, as reduction in rScore may stem from mere toxicity of the shRNA factor, rather than the knock down of AIMP1 receptor, each rScore was penalized by the shRNA toxicity. The higher the toxicity of a given shRNA, the smaller the difference between the given shRNA rScore and the rScore of AIMP1, which means, a weaker effect on the abrogation of AIMP1 rScore:

rScore_{i,j}=rScore_{i,j}+(rScore_{AIMP1}−rScore_{i,j})*toxicity_{i,j}
6. Calculating rScore fold change per gene knock down: for each gene, the rScore was averaged over all of its shRNA oligos, and the mean rScore was normalized to AIMP1 rScore. The lower this ratio, the stronger the abrogation of AIMP1 μmediated resistance.

$$r Score fold {change}_{gene X} = \frac{mean ( r Scores of sh RNA oligos )}{r {Score}_{AIMP 1}}$$

qRT-PCR—Total RNA was purified using Direct-Zol RNA mini-prep kit (Zymo-research, catalogue #R2053) according to the manufacturer's protocol. Two g of total RNA from each sample was reverse transcribed using Bio-RT (Bio-Lab, Cat #9597580273) and random hexamer primers. qRT-PCR was performed on a StepOnePlus real-time PCR System (Applied Biosystems) using KAPA SYBR Green Fast ABI Prism qPCR kit (BIOSYSTEM, Cat #020019566). Human TNF-alpha (PeproTech, 300-01A) was used to measure a possible shift in BRAFi resistance gene expression signature (34). Data analysis was performed according to the ΔΔCt method, by normalization of the expression level of each gene to that of beta-actin (ACTB) reference gene in the same sample.

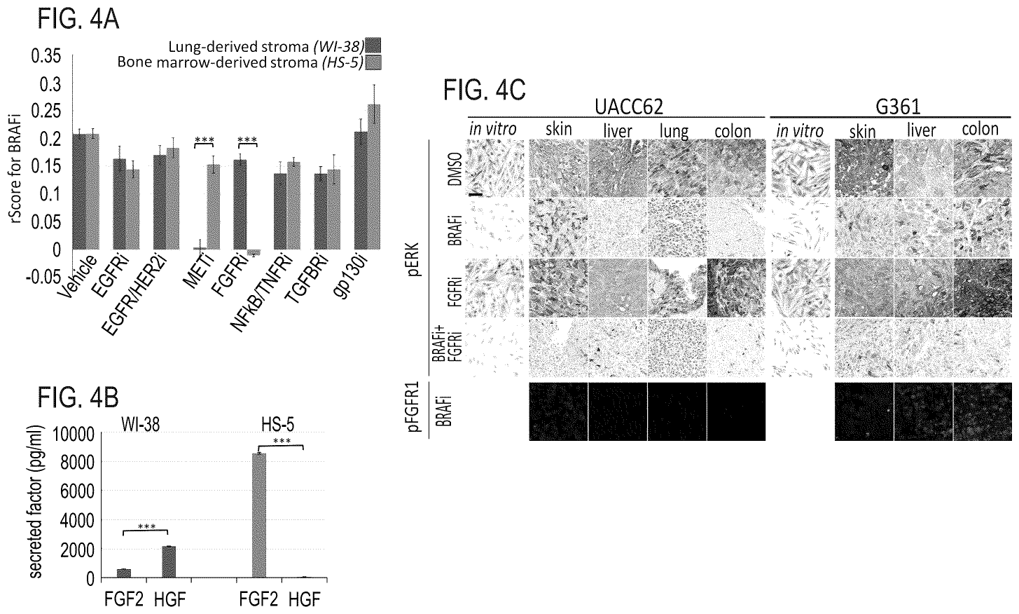

Co-culture of stroma and cancer cells—To demonstrate tissue-specific effects on innate drug resistance mechanisms (FIG. 4A), stromal cell lines of lung and bone marrow origin, known to mediate resistance to BRAF inhibition (5), were seeded in 384 wells plates, 1700 cells per well. Four hours later, the melanoma BRAF (V600E) mutated cell line SK-MEL-5 was seeded on top of the stromal cells at a concentration of 1700 cells per well, or seeded without stroma. Cells were treated with either DMSO, vemurafenib (2 μM), or vemurafenib in combination with 7 different inhibitors of potential resistance mechanisms: the MET inhibitor Crizotinib (0.3 μM), the FGFR inhibitor AZD4547 (0.05 μM), the NfkB inhibitor CAPE (10 μM), the EGFR/HER2 inhibitor lapatinib (0.01 μM), the TGFBR inhibitor LY2109761 (0.5 μM), the EGFR inhibitor gefitinib (0.1 M) and the gp130 inhibitor SC144 (0.001 μM). Concentrations of the inhibitors were based on dose curves with SK-MEL-5. For each inhibitor of potential mechanism of resistance, the maximal concentration with minimal effect (toxicity) on cell growth was selected (Cₘₐₓ).

To compare rScore value of stroma-mediated resistance with or without a given inhibitor, the rScore values of each inhibitor in the presence of stroma, was calculated. As a reduction in rScore may stem from mere toxicity of the inhibitor, rather than the abrogation of the stroma mediated resistance, each inhibitor rScore was penalized by its toxicity. The higher the toxicity of a given inhibitor, the smaller the difference between the given inhibitor rScore and the rScore of the stroma, which means, a weaker effect on the abrogation of stroma rScore:

rScoreinhibitor=rScoreinhibitor+(rScorestroma−rScoreinhibitor)*tOxicitY(Cₘₐₓ)inhibitor

ELISA—To quantify secreted FGF2 and HGF from stromal cell lines (FIG. 4B), an FGF2 (R&D, DFB50) or HGF (R&D, DHGOOELISA) ELISA assay was performed according to manufacturer instructions.

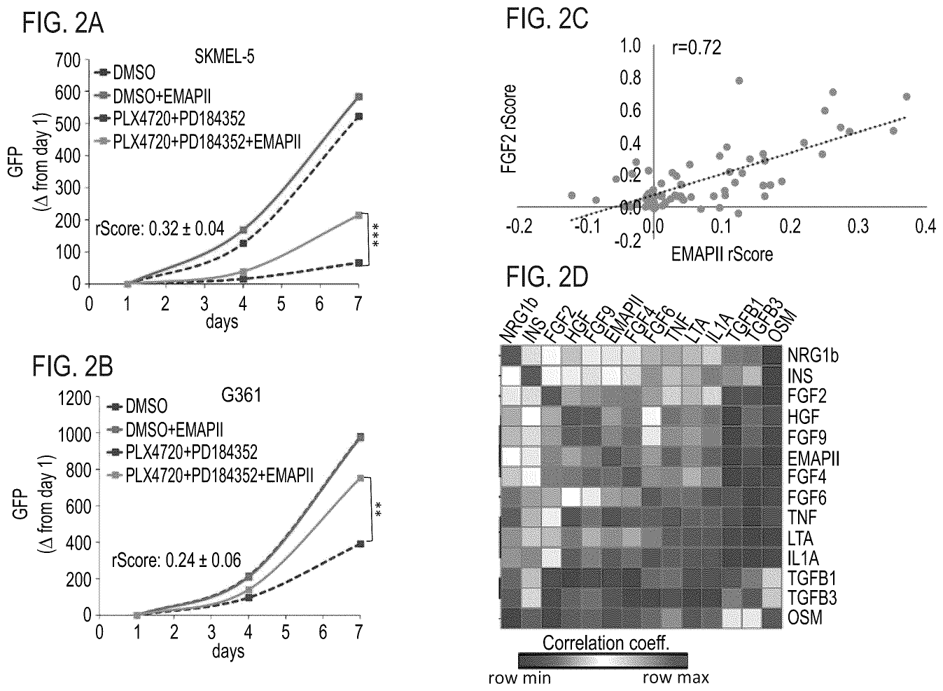

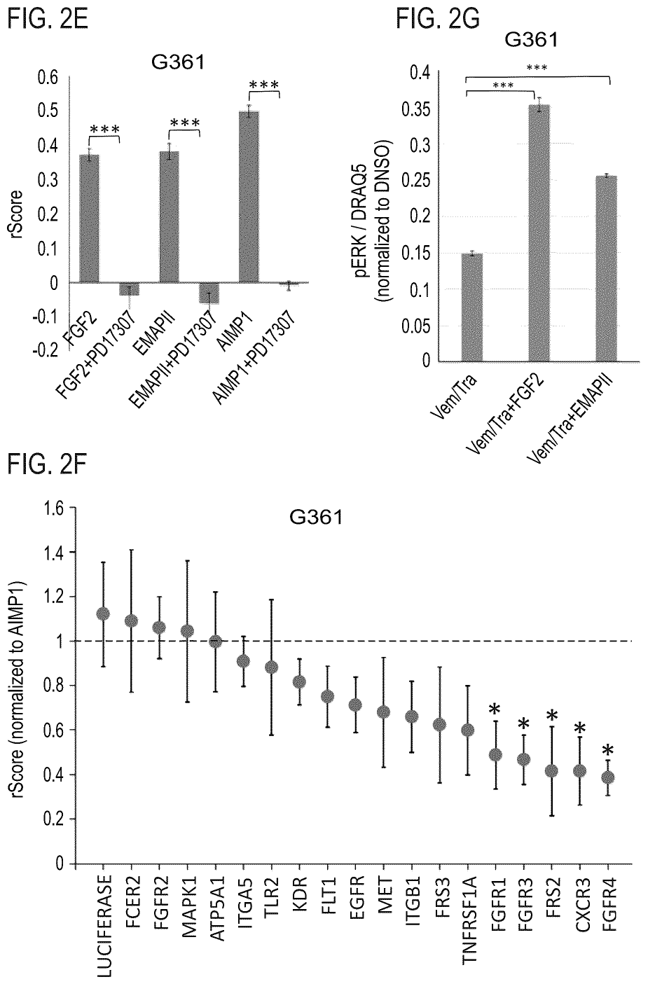

In-cell western blot—To validate that EMAPII mediates its effect on resistance to BRAF inhibition via the FGF receptors (FIG. 2G), the melanoma BRAF mutated cell line G361 was seeded in a 384-wells plate at a concentration of 16,000 cells per well. The following day, cells were treated with DMSO, or vemurafenib (2 μM)+Trametinib (1 nM) for 24 hours. Cells were either treated with EMAPII (200 ng/ml), human FGF2 (25 ng/ml) or vehicle control. Following medium removal and PBS wash, cells were fixated with 4% formaldehyde/0.1% Triton X-100 in PBS for 30 μminutes at room temperature. Following, the fixative was replaced, plate was washed with PBS and cells were blocked with Odyssey blocking buffer (Li-cor, 927-40000) for one hour at room temperature. Plate was emptied and cells were incubated overnight at 4° C. with pERK 1:1000, (Sigma, M8159) in Odyssey blocking buffer/0.1% TWEEN20. Next, the cell plate was washed 3 times with DDW/0.1% TWEEN20, followed by incubation with the secondary antibody, IRDye 800CW Goat anti-Mouse IgG (H+L) (Li-cor, LIC926-32210), diluted at 1:800 in Odyssey blocking buffer/0.1% TWEEN20/0.1% SDS. To normalize for total protein level, secondary antibody solutions were supplemented with DRAQ5, 1:10,000 (abeam, ab108410). Following 1 hour incubation while shaking at room temperature, the plate was washed three times with DDW/0.1% TWEEN20, then washed once with PBS to remove bubbles. Finally, the plate was scanned with Odyssey (Li-cor) using microplate settings (169 μm, 3 μm focus), and fluorescence intensity was quantified and normalized to the DRAQ level, and then to no drug control (DMSO).

RNA-seq datasets and analysis—To characterize the variability in expression level of secreted factors that were found to potentially confer drug resistance across different cancer types, several public databases were used. Expression data of Melanoma BRAF (V600E) mutated cell lines was retrieved from CCLE (portals(dot)broadinstitute(dot)org/ccle). RNA-Seq expression data of human melanoma BRAF (V600E) was retrieved from The Cancer Genome Atlas (TCGA) (cancergenome(dot)nih(dot)gov/). RNA-Seq expression data of human breast tumors was retrieved from TCGA. RNA-seq expression data (Affymetrix) of human NSCLC EGFR mutated cohort was retrieved from GEO (GSE31210). To compare the expression of secreted factors that were found to potentially confer innate resistance to BRAF inhibition in melanoma pre- and post-treatment, the following patient cohorts of melanoma tumor pre- and early on treatment with BRAF/MEK inhibitors were used: The “Kwong” dataset (44), the “Van Ellen” dataset (56) and the “Miles” dataset (43).

Immunohistochemistry of tumor microarray (TMA) of melanoma BRAF (V600E) mutated patients—To characterize the variability of selected secreted factors that were found to confer innate resistance in melanoma, TMA containing 36 BRAF mutated melanoma patients (2 cores per patients) was used. The TMA was stained and scanned with Vectra 3.0 (PerkinElmer) at the HTSR (lombardi(dot)georgetown(dot)edu/research/sharedresources/htsr). Multiplexed IF staining was used to stain the TMA for anti-AIMP1 N terminal (Sigma, SAB2502063), anti-NRG1 (Spring Biosci., M4420), anti-TGFB3 (abeam, ab15537), anti-FGF9 (Santa-Cruz, sc-8413) and PanMel (Biocare, CM165B). Tissues from Pancreas, GBM, Placenta, G B M and Melanoma, respectively, served as positive controls. A second TMA slice was stained with anti-LTA (Sigma, HPA007729), anti-FGF2 (abeam, ab8880), anti-pFGFR1 (abeam, ab59194), anti-CCL4 (abeam, ab9675), anti-HGF (Acris, TA807186) and PanMel (Biocare, CM165B). Tissues from Spleen, GBM, NSCLC, Tonsil, H C C and Melanoma, respectively, served as positive controls. TMA signal was quantified semi-automatically by a Matlab GUI. Briefly, per core, regions of interest (ROI) were determined manually by a polygon to exclude core margins, tissue folds and holes in order to avoid staining biases. Next, per core ROIs, per channel, mean signal intensity was calculated.

Ex-Vivo Organ Culture (EVOC)—EVOC protocol: To demonstrate, ex vivo, the prioritization of co-targeting innate resistance mechanism, immunocompromised mice bearing human tumors or human biopsies were used. Freshly resected tumors were cut to 250 μm slices (Compresstome, VF-300) in cold Williams-E medium (Sigma, W1878). Slices were placed on the surface of titanium meshes which were pre-incubated in DMEM/F-12 (HAM) (01-170-1A, BI) supplemented with 10% FCS, 100 units/ml Penicillin and Streptomycin, 2 μmM Glutamine, 50 g/ml gentamicin, and 2.5 μg/ml Amphotericin B (sigma, A2942), at 37° C. and 80% oxygen/5% CO2. Timeline of experiment: day 0: slices equilibration, days 1-4: treatment. Note: media was replaced to fresh media & drug after 2.5 days of treatment. Following 4 full days of treatment, slices were fixated with 4% PFA, embedded in paraffin and used for generating FFPE blocks. Liver tissue required additional medium oxygenation with a mixture of 95% 02/5% CO2, by a dispersion gas tube (Sigma, CLS3952530C-1EA) for 30 μminutes on days 0 and day 2.5 (before each media change).

Immunohistochemistry of EVOC tissues: To assess response therapy, 4 μm FFPE tissue slices were stained with H&E, or with specific antibodies: anti-pERK (Cell Signaling, #4370) followed by HRP conjugated secondary antibody (anti-Rabbit HRP, ZUC032, ZytoChem) and DAB staining (DAB substrate kit, DAB057, ZytoChem). Anti-pFGFR1 (abcam, ab59194) or anti-pHER3 (Cell Signaling, #2842) were used, followed by secondary antibody Alexa fluor 647 (Thermo, A21245). All slides were scanned using the Pannoramic SCAN II (3DHISTECH) and analyzed by a pathologist. The percentage of viable cancer cells was morphologically assessed on H&E stained sections by a pathologist as the ratio between viable cancer cell area and total cancer area (viable cancer cells plus necrotic cancer cells). As the immediate samples often showed areas of coagulative necrosis, for this purpose only colliquative necrosis was taken into account whereas coagulative necrosis was excluded.

Unsupervised hierarchical clustering—Euclidean distance of tumor samples was carried out using GENE-E (www(dot)broadinstitute(dot)org/cancer/software/GENE-E/).

Statistical analysis—Per experiment, similar processing was applied to all groups. Number of replicates and statistical tests are indicated in the brief description of the Figures hereinabove. In the in-vitro experiments outliers related to hardware malfunction (e.g. pipetation errors, biased fluorescence reading) were discarded. In the in-vivo mice experiments, outliers related to failure in injecting cancer cells were discarded.

Statistical parameters including the exact value of n, the definition of center, dispersion and precision measures (mean±SE) and statistical significance are reported in the Figures and Brief description of drawing hereinabove. Appropriate statistical tests and p-values are reported as well. In case of multiple hypotheses, the Q-value was denoted following Benjamini-Hochberg procedure for controlling the FDR. In figures, asterisks denote statistical significance (*, p<0.05; **, p<0.01; ***, p<0.001). Statistical analysis was performed in GraphPad PRISM 6 or matlab. To calculate the probability of getting the expression trend of N genes {G₁, G₂ . . . Gₙ} with respect to two sub-groups (groupA, groupB) (FIG. 1H), the following Monte-Carlo simulation was applied. Briefly, per gene, median expression and Zscore was calculated on the entire group of 185 μmelanoma patients {Z(G₁), Z(G₂) . . . Z(Gₙ)}. The delta of each subgroup Zscore was calculated per gene (delta=mean groupA(Z(Gₙ))—mean groupB(Z(Gₙ)). The minimal positive delta of the N genes was set as a threshold for the Monte carlo test. Per gene (Gₙ), a pull was composed from genes (out of the entire genome, excluding the gene input list) with similar median expression (30 most similar genes). Per trial (out of K=1000 trials), one gene was randomly drawn from each of the N pulls. A given trial was counted (k′) if the minimal delta of the N randomly drawn genes was greater or equal to the threshold delta. P-value is the ratio K′/K. Simulation and statistical analysis were performed in Matlab.

### Example 1

### Multiple Secreted Factors Mediate Innate Resistance or Sensitivity to Anti-Cancer Drugs

To systematically characterize the potential of TME-mediated innate drug resistance, a library of 321 recombinant proteins, which were prioritized by their degree of secretability (20) and their known expression in human tumors, was assembled. The proteins in the generated secretome library included growth factors, immune factors, endocrine factors, extra-cellular matrix related factors, and others (FIG. 1A, Table 2 hereinabove). The final concentration of the factors was determined based on the reported range of ED50 concentration and on the solubility limit. Following, the effect of each of these factors on the sensitivity of 59 GFP-labeled cancer cell lines to 35 clinically relevant cytotoxic and targeted anti-cancer therapies was determined. The cancer cell lines that were chosen represent eight different common solid tumor types, including melanoma (29), non-small cell lung (7), ovarian (7), breast (5), pancreatic (3), esophageal (3), colorectal (3), and prostate (2) cancers. Drug concentrations were determined by preliminary experiments finding the EC90 of growth inhibition for each drug-cell line pair. The effect of the secreted factors was determined by reading GFP fluorescence from the cancer cells over 7 days of treatment (FIGS. 1B-D). Further, the effects of the secreted factors were calculated both on the proliferation rate of all cancer cell lines (pScore, FIGS. 8A and 8C) and on the sensitivity of the cancer cell lines to drugs. The factors which demonstrated the strongest effects on proliferation included many known pro-proliferative secreted factors (e.g., insulin (21) and neuregulin-1 (22)) and anti-proliferative secreted factors (e.g., TGF-beta (23) and interferon gamma (24), FIG. 8D). Moreover, while some secreted factors mediated drug resistance, others enhanced anti-cancer drug activity. Therefore, two different scoring systems were used interchangeably, based on the secreted factor effect. For secreted factors that conferred drug resistance, a rescue score (rScore) that reflects the fraction of drug effect that is lost in the presence of the factor was calculated (FIGS. 8A and 8C). For secreted factors that enhanced drug efficacy, a Bliss score (25) (bScore, FIGS. 8B and 8C) was calculated to quantify the synergistic effect between the drug and secreted factor.

Overall, a total of 278 screens encompassing 70,688 unique experimental conditions was performed; and the results of the screens were merged into 21 groups based on the cancer type, drug target and the similarity between the screens' vectors of rScore values (data not shown). Following, the effect of each factor on each group was collapsed into 4 ranks of either resistance or synergism. Ranks were determined based on the number of cell lines whose drug sensitivity was affected by the factor and the magnitude of the effect (FIGS. 9A-D). Thirteen groups that contain at least 4 screens are shown in FIG. 1E.

In the broad perspective, secreted factors had a stronger effect on the sensitivity of cancer cells to targeted therapies than to cytotoxic drugs (FIG. 1E). This is in agreement with previous work characterizing stromal cells-mediated chemoresistance (5). In addition, of the 321 tested factors, the repertoire of factors that can mediate drug resistance was limited largely to RTK ligands, TNF pathway ligands, and TGFβ pathway ligands. At the single factor level, multiple factors whose effect on drug sensitivity is well established (e.g., HGF(26), NRG1(27) and FGF2(28)) were recovered. In addition, for several factors there is no previous documentation on their effect on the sensitivity to the targeted therapy tested in the screens e.g. prolactin, oncostatin, endothelial-monocyte activating polypeptide II (EMAPII), and fibroblast growth factors 7 and 10 (FGF7 & FGF10).

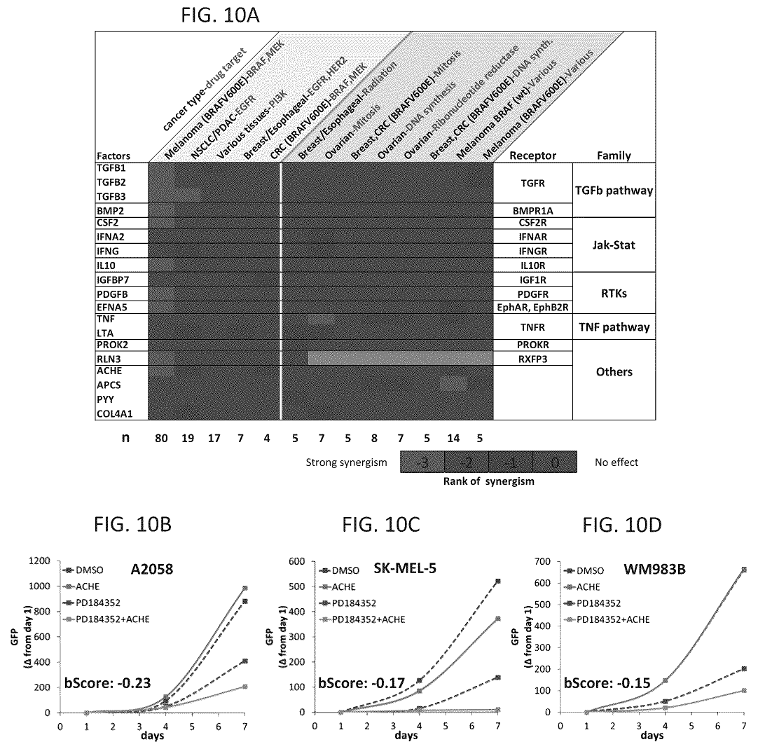

Moreover, multiple factors that have a synergistic effect with clinically relevant anti-cancer drugs were uncovered (FIG. 10A). For example, acetylcholinesterase (ACHE) exhibited synergistic effect with BRAF and MEK inhibition in multiple melanoma cell lines (FIGS. 10B-D). This result is consistent with the known anti-proliferative (29,30) and pro-apoptotic (31) effects of ACHE on cancer cells, and with its down-regulation in several cancer types (27,28).

Following previous work demonstrating differential drug effects between 2D and 3D cultures (33), the observed effects were also tested in a simplified model of 3D culture, using droplet-derived PEG micro-tissues. To this end, the effect of the secreted factors on the sensitivity of the BRAF-mutated G₃₆₁ human melanoma cell line to BRAF inhibition was evaluated. Overall the main secretome screen results in were recapitulated in the 3D culture (FIG. 1F).

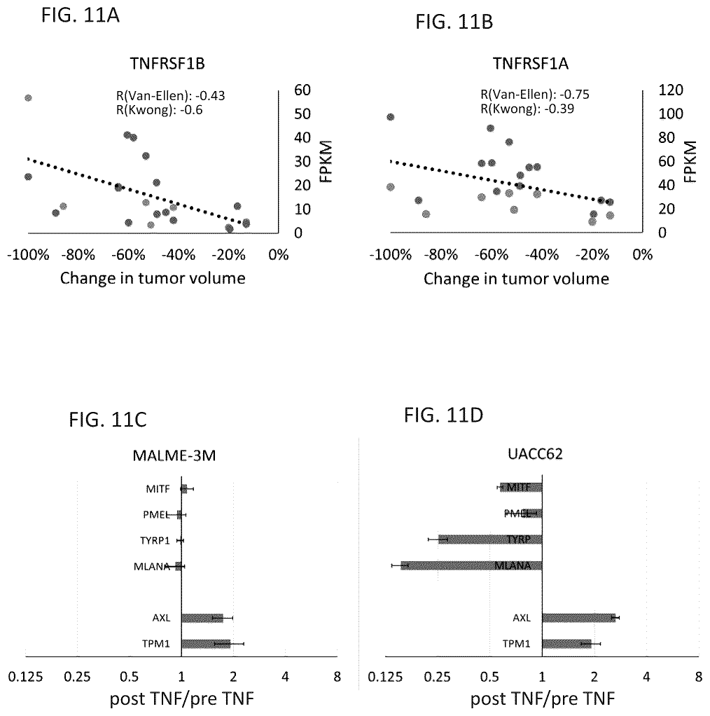

To demonstrate the clinical relevance of the findings, the present inventors asked whether tumors with high expression of resistance mediating factors are more resistant to drug therapy than tumors with relatively low expression of those factors. To this end, an eight-gene expression signature that was demonstrated to be an accurate biomarker for the response of melanoma to BRAF/MEK inhibition (34) was used. Unbiased clustering of 185 BRAF-mutated melanoma patients in the TCGA based on their eight-gene expression signature identified 26 patients with a strong resistance signature and 67 patients with a strong sensitivity signature (FIG. 1G). Almost all the secreted factors found to mediate resistance to BRAF inhibition had a higher expression level in the group of drug-resistant patients (FIG. 1H). The only factors that did not follow this trend were tumor necrosis factor alpha (TNF-α) and lymphotoxin alpha (TNF-b). Interestingly, high expression of their receptors, TNFRSF1A and TNFRSF1B, was also found to be associated with better response of melanoma patients to BRAF inhibition (FIGS. 11A-B). The present inventors speculated that this is the result of TNFa-mediated intra-tumor inflammation, a component that is lacking the in-vitro screen utilized. Thus, the screen uncovered the direct effect of TNFa on cancer cells, which may be minor compared to the immune-mediated effects of TNFa in-vivo. To further support the direct effect of TNFa on melanoma cells, the addition of TNFa to the UACC62 μmelanoma cell line can induce a shift in the eight-gene signature toward resistance (FIGS. 11C-D).

Zooming in on one of the factors with a previously unrecognized effect on drug resistance, the drug resistance mechanism mediated by EMAPII was further deciphered. EMAPII is generated by cleavage of the aminoacyl tRNA synthetase complex interacting multifunctional protein 1 (AIMP1), and corresponds to its C-terminus. AIMP1 is known to regulate the loading of amino acids to tRNAs by tRNA synthetases, and can also function as a bona fide secreted cytokine, either in its full length (AIMP1) or by its C-terminus variant (EMAPII) (35). AIMP1 and EMAPII can bind to multiple receptors such as the Fc fragment of IgE receptor II (FCER2), fibroblast growth factor receptor 2 (FGFR2), C—X—C motif chemokine receptor 3 (CXCR3), Fms related tyrosine kinase 1 (FLT1), alpha subunit of ATP synthase (ATP5A1), Alpha 5 beta 1 integrin (ITGA5 and ITGB1), TNF receptor superfamily member 1A (TNFRSF1A) and Toll-like receptor 2 (TLR2).

To validate the screen results, the EMAPII effect on the response of two BRAF-mutated melanoma cell lines to BRAF/MEK inhibition was retested. In full agreement with the screen, EMAPII conferred resistance to BRAF and MEK inhibition in both G₃₆₁ and SK-MEL-5 cell lines (FIGS. 2A, B). Addressing which receptors are involved in EMAPII-mediated resistance to BRAF/MEK inhibition, it was found that EMAPII and FGF2 were highly correlated in their rScore values across all 25 BRAF-mutated melanoma cell lines, suggesting that they may have a similar mechanism of action (FIG. 2C). In addition, unsupervised clustering of the factors mediating drug resistance by their correlation of rScore values, across all melanoma BRAF mutated cell lines, clustered EMAPII together with the FGF ligands (FIG. 2D). Therefore, it was hypothesized that the EMAPII effect is mediated by activation of the FGFR signaling pathway. In agreement with this hypothesis, the effect of both EMAPII and AIMP1 on the resistance of G₃₁ μmelanoma cells to BRAF inhibition was completely abrogated by the addition of FGFR inhibitor (FIG. 2E). Further, the effect of knocking down each of the known potential AIMP1 receptors by shRNA was measured. Consistent with the suggested hypothesis, of the 5 top receptors with the strongest effect on AIMP1-mediated resistance to the BRAF inhibitor PLX4720, four belonged to the FGFR pathway: FGFR1, FGFR3, FGFR4, and FGFR substrate 2 (FRS2) (FIG. 2F). The knock down of FGFR2 had no effect on AIMP1-mediated resistance, probably because of its low expression level in melanoma (based on CCLE dataset). Finally, similarly to FGF2, the addition of EMAPII or AIMP1 to G₃₆₁ cells that were treated with the BRAF inhibitor can partially reactivate pERK (FIG. 2G). Overall, the results demonstrate that AIMP1 can affect the sensitivity of BRAF-mutated melanoma cell lines to BRAF/MEK inhibition by direct activation of FGFR signaling.

### Example 2

### The Complexity in Clinical Implementation of Co-Targeting the Identified Innate Mechanisms of Drug Resistance

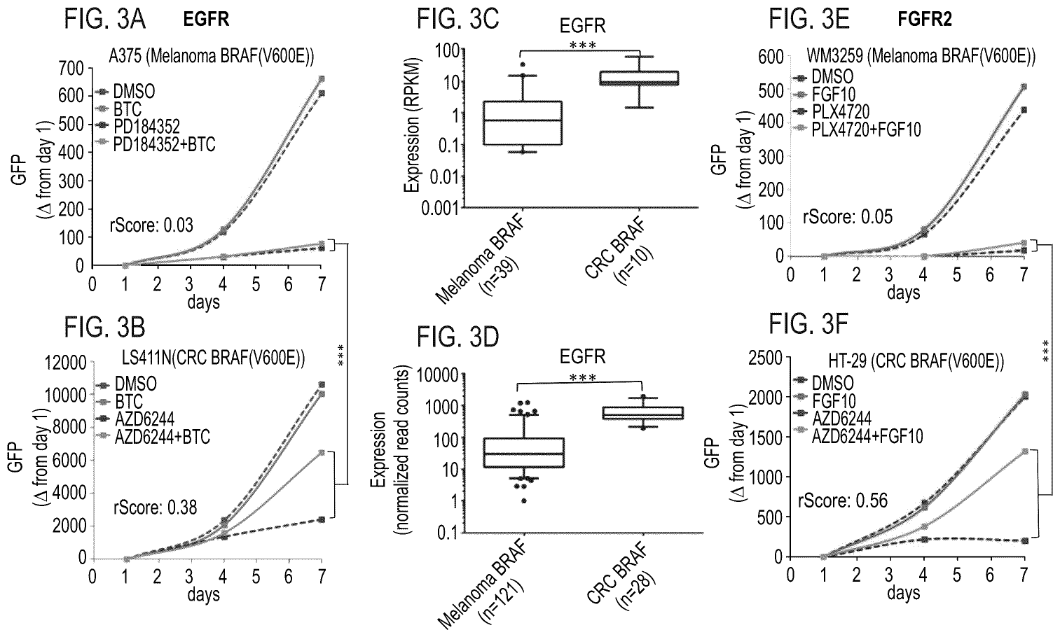

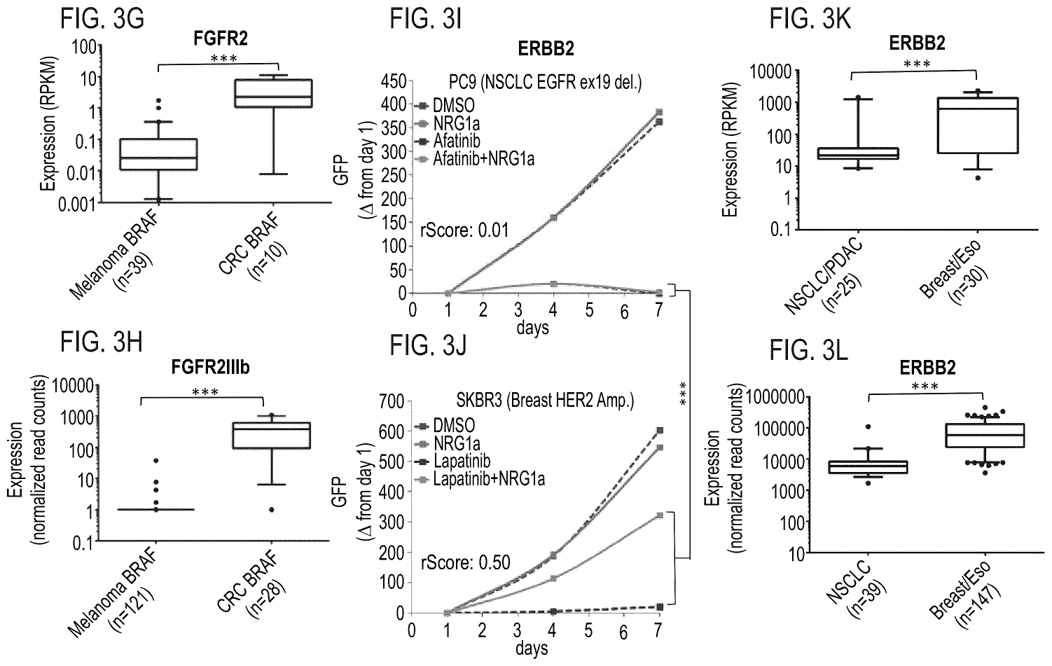

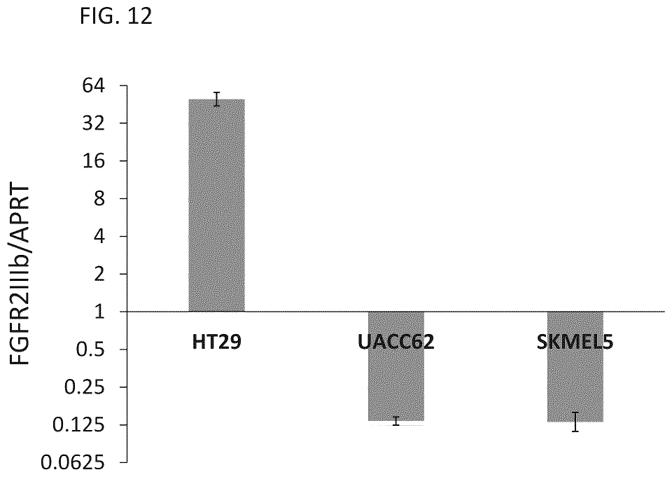

As described in details hereinabove, the screen demonstrated that unique sets of factors can potentially confer drug resistance to different cancer types (e.g. FIG. 1E); however this cancer type-specific effect can be frequently attributed to the availability of receptors on the cancer cells. In the case of the BRAF (V600E)-mutated melanoma and colorectal cancer cell lines, EGFR ligands (e.g. beta-cellulin (BTC)) were found to mediate resistance only to the colorectal cell lines (FIGS. 1E and 3A-B). Indeed, expression data from both cancer cell lines and patient tumors demonstrate that EGFR expression level is significantly higher in BRAF (V600E) colorectal cancer (FIGS. 3C-D). This is in agreement with previous reports demonstrating that colorectal but not melanoma BRAF (V600E) cancer cells require the addition of EGFR inhibitors to overcome their innate resistance to BRAF inhibition (36,37). A similar dichotomy was shown by the differential effect of FGF7 and FGF10 on cancer cell lines bearing the BRAF (V600E) mutation. Although these ligands could confer drug resistance to colorectal cancers, they had a much smaller effect on melanoma cell lines (FIGS. 1E and 3E-F). Both FGF7 and FGF10 are known ligands of FGFR2IIIb—a splice variant of FGFR2 (38). It was found that the expression level of the FGFR2IIIb isoform is significantly higher in BRAF (V600E) colorectal human tumors than in BRAF (V600E) human melanoma tumors (FIG. 3G). While data regarding the expression of the FGFR2IIIb isoform in the CCLE database is lacking, the total expression level of FGFR2 isoforms was shown to be higher in BRAF (V600E) colorectal cancer cell lines than in melanoma BRAF (V600E) cell lines (FIG. 3H). Finally, using primers that are specific to the FGFR2IIIb isoform, its expression was found to be a 100-fold higher in the HT29 colorectal cancer cell line than in UACC62 and SK-MEL-5 melanoma cell lines (FIG. 12). In the case of neuregulin-1 (NRG1)-mediated resistance to EGFR and HER2 (ERBB2) inhibitors, the present inventors found that although NRG1a can mediate resistance to breast and esophageal cancers, it has no effect on lung and pancreatic cancers (FIGS. 1E and 3I-J). By contrast, NRG1p had a ubiquitous effect and could mediate resistance to EGFR/HER2 inhibitors across all cancer types tested. The difference between the effect of the NRG1 isoforms may be attributed to the 100-fold higher affinity of NRG1(3 to the NRG1 receptors ERBB3 and ERBB4 relative to NRG1a (39). As the expression level of ERBB2 is much higher in breast and esophageal cancers (FIGS. 3K-L), and as dimerization of ERBB2 with ERBB3/ERBB4 increases NRG1 affinity to these receptors (39), it is likely that NRG1a is active only when ERBB2 is highly expressed. By contrast, lower expression of ERBB2 in lung and pancreatic cancers results in lower affinity of ERBB3/ERBB4 to NRG1, which can then be activated only by the NRG1(3 isoform. Overall, differences in the relevant receptor levels may account for some of the variability in the potential of secreted factors to confer drug resistance to different cancer types, as was also suggested by others (9,40).

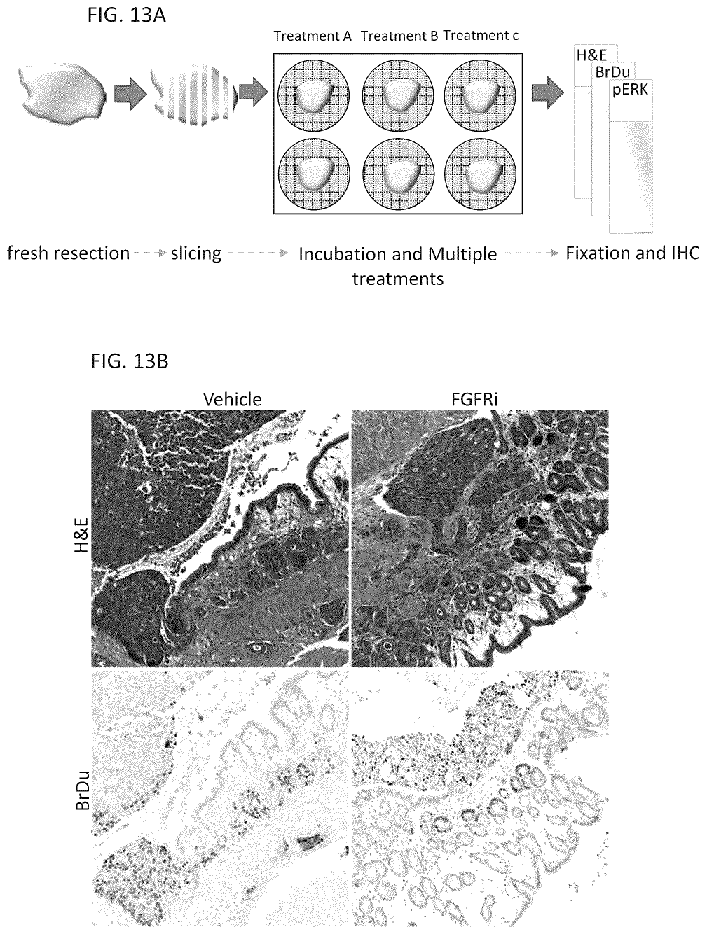

Further, while the screen results portrays the landscape of potential mechanisms of innate drug resistance that can affect different tumor types (e.g. FIG. 1E); in-vivo different subsets of these mechanisms may come into play in different anatomical locations. For example, as different tissues possess a unique set of secreted factors, it was hypothesized that the same cancer cells may benefit from different mechanisms of innate resistance, depending on their tissue-specific location. To model, in-vitro, tissue-specific effects on innate drug resistance, the BRAF (V600E) mutated melanoma cell line SK-MEL-5 was co-cultured with two different stromal cell lines originating from different tissues. Both the lung-derived stromal cell line, WI-38, and the bone-marrow derived stromal cell line, HS-5, conferred resistance to the BRAF inhibitor vemurafenib (rScore>0.2 for both, FIG. 4A). Following, based on the screen results (FIG. 1E), the potential mechanisms of resistance were co-targeted to try to abrogate the stroma-mediated resistance. Whereas co-targeting the HGF receptor MET by crizotinib abrogated the resistance effect of WI-38, co-targeting of FGFR by AZD4547 was needed to abrogate the resistance effect of HS-5. Correspondingly, whereas the WI-38 cell line secrets large amounts of the MET ligand HGF, the HS-5 cell line secrets large amounts of the FGFR ligand FGF2 (FIG. 4B). To model tissue-specific effects on the drug sensitivity of genetically identical tumors in-vivo, xenograft models of BRAF-mutated melanoma tumors were generated in the skin, liver, lung, and colon of mice, using UACC62 and G₃₆₁ cell lines. When the tumors reached a volume of −700 μmm³, they were resected, sliced into 250 μM slices, and cultured ex-vivo for 4 days, without any visible damage to the tissue viability or proliferation capacity (FIG. 13A). These slices preserve the 3D structure of the original tumor, and contain, in addition to cancer cells, the original components of the TME, including stromal cells and the extracellular matrix. Following incubation with either vemurafenib or vehicle control, the effect of treatment was assessed by IHC of pERK levels in the cancer cells, as ERK was shown to be inhibited by BRAF blockade and partially reactivated by different mechanisms of innate resistance (5,9). Although BRAF inhibition was sufficient to inhibit pERK in UACC62 tumors in the liver, lung, and colon, only partial pERK inhibition was observed in skin tumors (FIG. 4C). In the G₃₆₁ tumors, BRAF inhibition could not completely suppress pERK in any of the models tested (FIG. 4C). The lack of innate resistance to BRAF inhibition in-vitro in both cell lines (FIG. 4C) further supports the critical role of the TME in mediating this resistance. To dissect the underlying TME-mediated resistance mechanisms, UACC62 skin tumors were treated ex-vivo with a combination of vemurafenib and drugs that target each of the potential mechanisms of resistance. The addition of the FGFR inhibitor AZD4547 to vemurafenib consistently achieved near complete inhibition of pERK (FIG. 4C). This is in accordance with previous reports demonstrating the role of FGFR in innate resistance to BRAF inhibition of melanoma tumors (41). Co-treatment of G₃₆₁ tumors with vemurafenib and AZD4547 was also sufficient to downregulate pERK in all of the different tumor locations, supporting the involvement of FGFR in the incomplete response of G₃₆₁ tumors to vemurafenib (FIG. 4C). Consistent with these observations, activation of the FGFR, as measured by IHC of pFGFR1, highly correlated with lack of complete downregulation of pERK in response to vemurafenib (FIG. 4C).

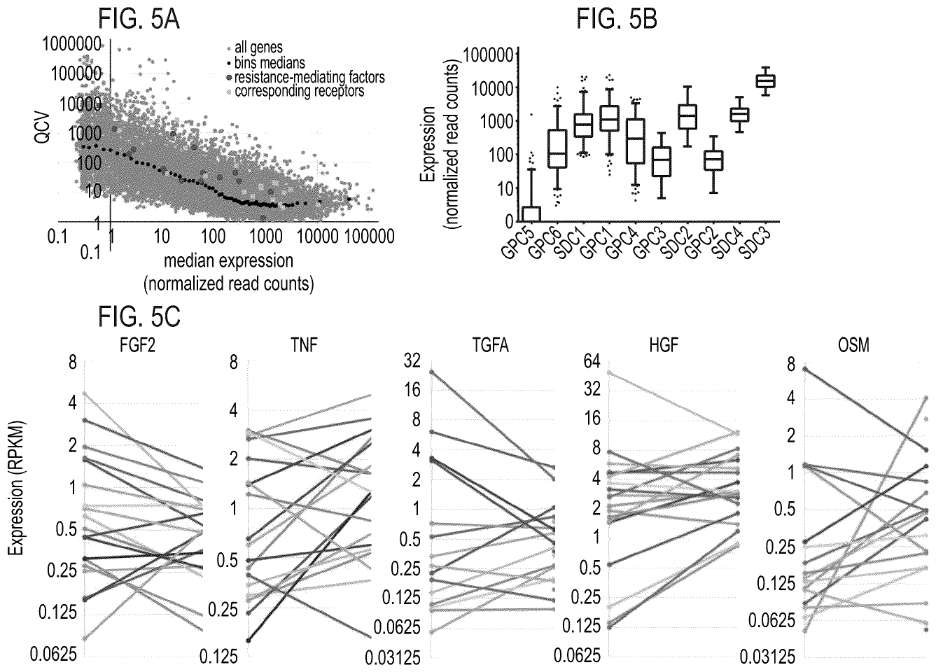

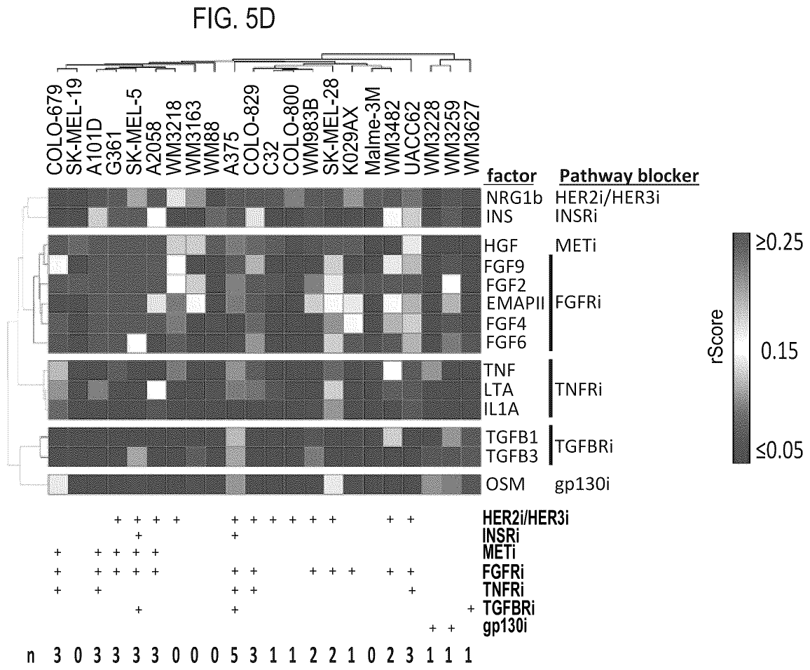

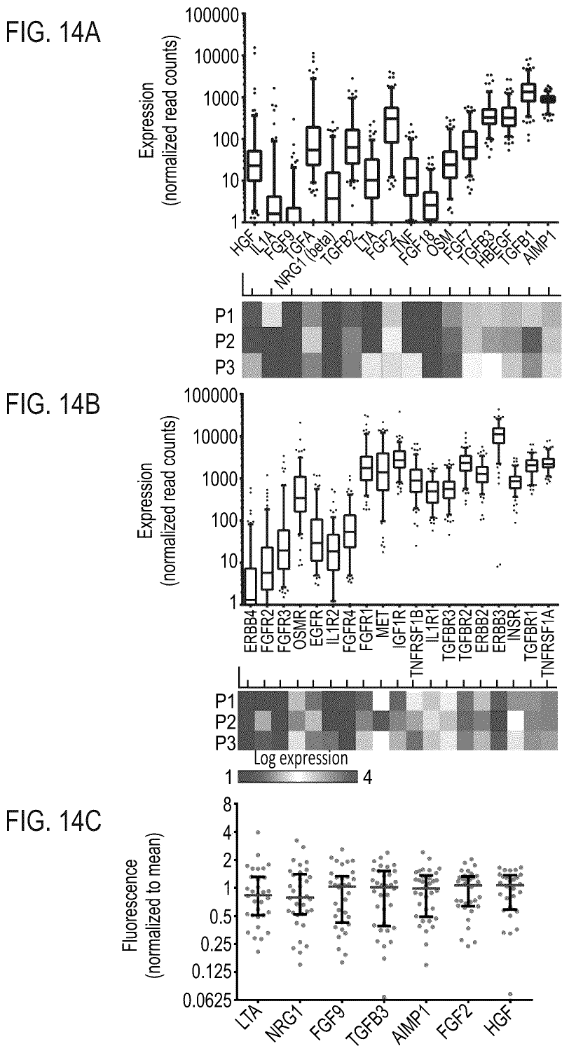

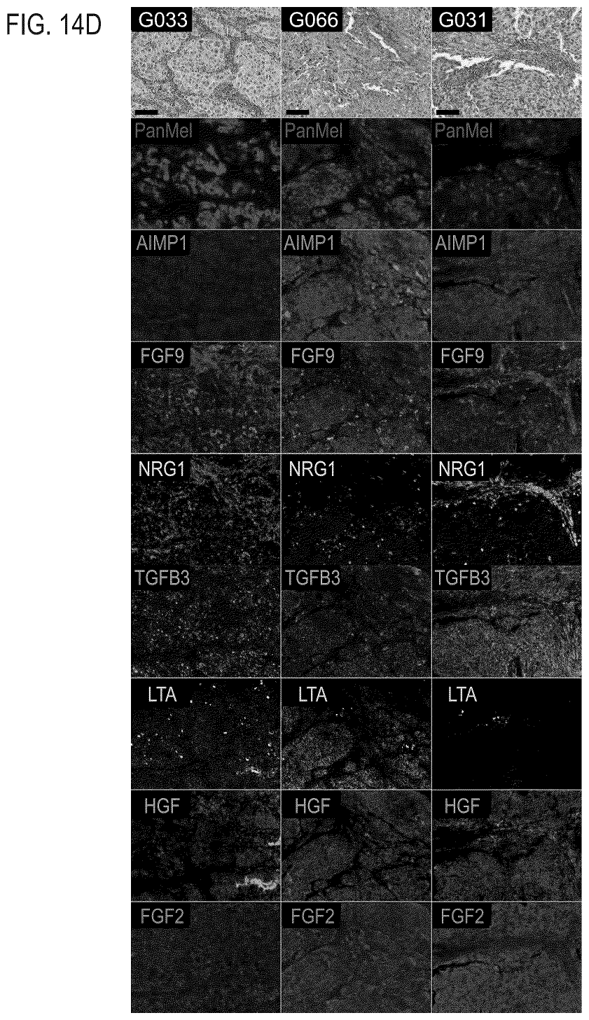

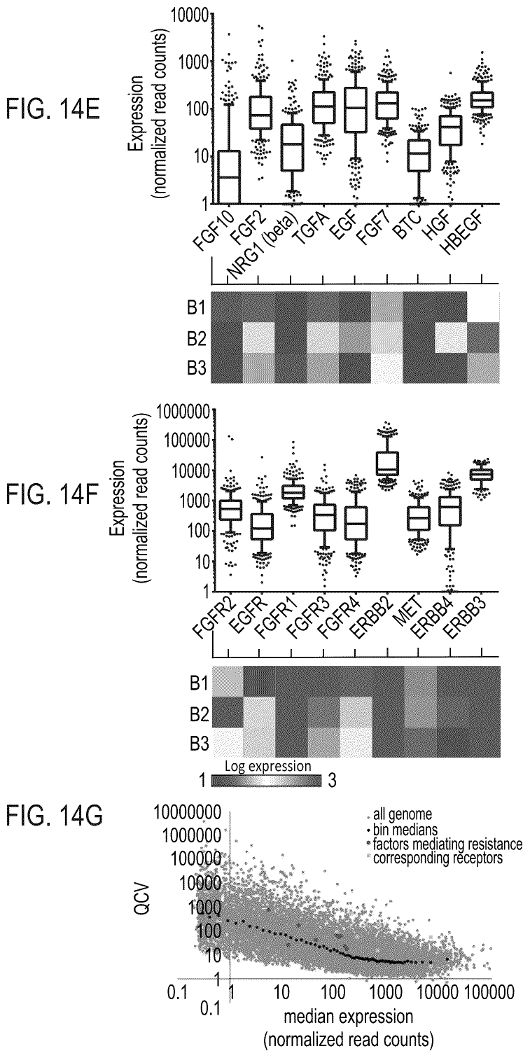

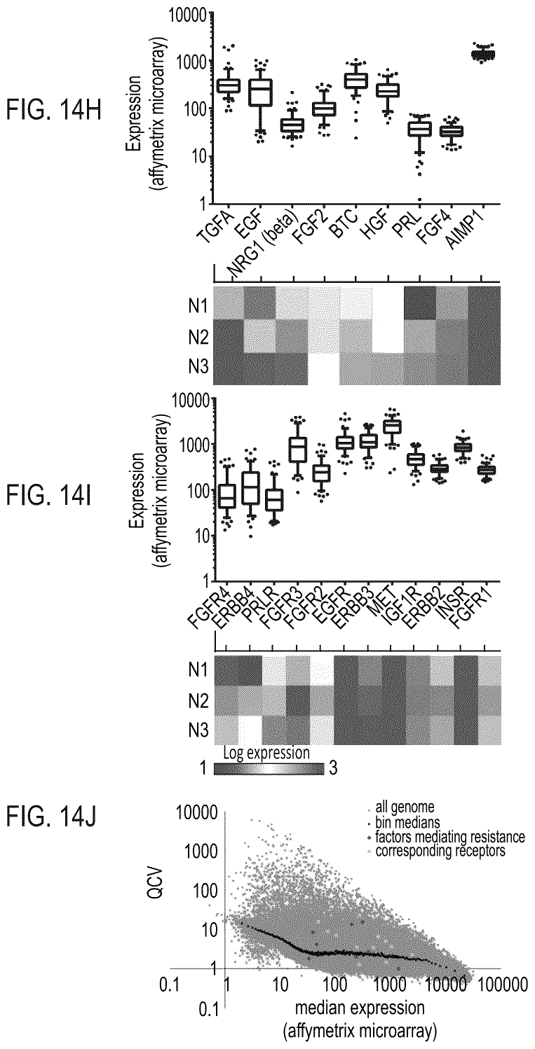

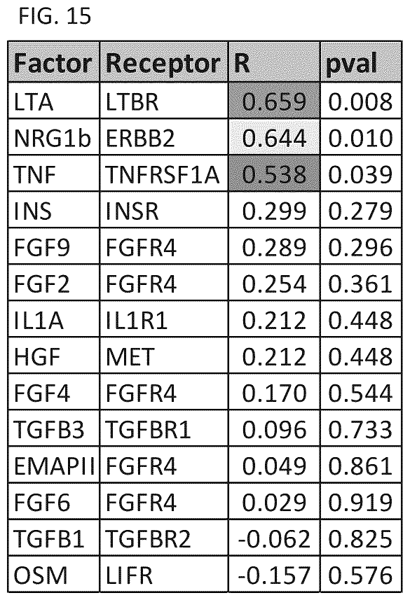

Overall, following the identification and characterization of cancer-type specific innate resistance mechanisms, this knowledge could be readily integrated for tailoring drug combinations by co-targeting genetic susceptibilities and tumor-specific mechanisms of resistance. Yet, the results also indicate that it is still difficult to predict, for any given patient and tumor, which of the potential mechanisms of resistance should be co-targeted to achieve a clinical benefit. Detailed below five levels of complexity that may interfere with finding the right drug combination:
(1) The expression level of resistance-mediating factors and their receptors are highly variable between patients with the same tumor type (FIGS. 14A-J). At the transcript level, this variability is almost always higher than the median variability expected by any gene with a similar expression level (FIG. 5A). Implementing integrative combined therapy would thus require quantification of a large number of transcripts or proteins. Clinical grade quantification of so many proteins may not be readily achievable.
(2) ECM components can affect the bioavailability of resistance-mediating factors. For example, the bioavailability of FGF2 μmay be affected by ECM components, such as heparan sulfate proteoglycans (HSPG), glypicans, and syndecans, which modulate FGF ligands binding to FGFR (42). The high variability of these factors between tumors further impedes the ability to predict the most significant patients-specific resistance mechanisms (FIG. 5B).
(3) The expression level of secreted factors and their receptors might change significantly upon treatment. Indeed, RNA-Seq from re-biopsy of 19 μmelanoma tumors 3-8 weeks on treatment with BRAF inhibitors (43, 44) demonstrated vast changes in their expression levels (FIG. 5C). Therefore, an on-treatment biopsy may be needed to accurately capture the relevant tumor-specific resistance mechanisms.
(4) The activity of receptors may be modulated by genetic alterations, such as mutations and amplifications, regardless of the presence of ligands. For example, HER2 and MET amplification can mediate the activation of these receptors and contribute to drug resistance even in the absence of their ligands (45,46).
(5) Even among cell lines of similar origin, a significant variability in the potential of secreted factors to mediate drug resistance was still observed (FIG. 5D). Of note, secreted factors were given in excess for all cell lines, and receptors level could not account for most of the inter-cell line variability (FIG. 15). It is therefore likely that other sources of variability (e.g., the downstream signaling of receptors, apoptotic machinery, efflux pumps) that cannot be readily measured may account for the observed differential effect of the secreted factors.

### Example 3

### Ex-Vivo Organ Culures (EVOCS) can be Used to Select Clinical Co-Targeting of Innate Mechanisms of Drug Resistance

The present inventors suggest that ex-vivo organ cultures (EVOCs) address the complexity involved in predicting tumor-specific mechanisms of innate drug resistance. As the EVOC slices preserve the original tumor composition and structure, they retain many of the potential mechanisms of innate resistance, thereby allowing the prioritization of drug combinations that co-target the tumor-specific mechanisms of innate resistance. Of note, while EVOC has a limited throughput when testing multiple drug combinations on a single tumor, the secretome screen enabled narrowing down the possible drug combinations to the most relevant resistance mechanisms per cancer type and treatment. In the majority of cases, up to three drugs were sufficient to overcome the relevant potential mechanisms of resistance for a given tumor-drug combination (FIG. 5D).

To demonstrate the feasibility of implementing integrative combined therapy, EVOC was first used to prioritize drug combinations for the treatment of preclinical cancer models, representing four cancer types: melanoma, colorectal cancer, lung cancer, and esophageal cancer. In the next step, the feasibility of prioritizing drug combinations using human tumor biopsies was effected.

### BRAF-Mutated Cancer Models

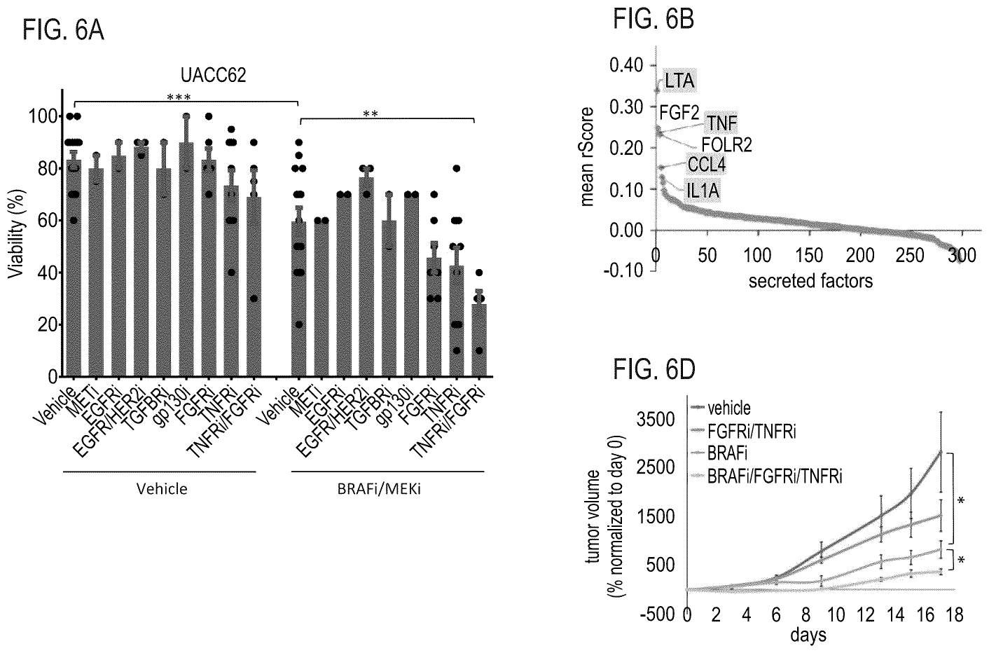

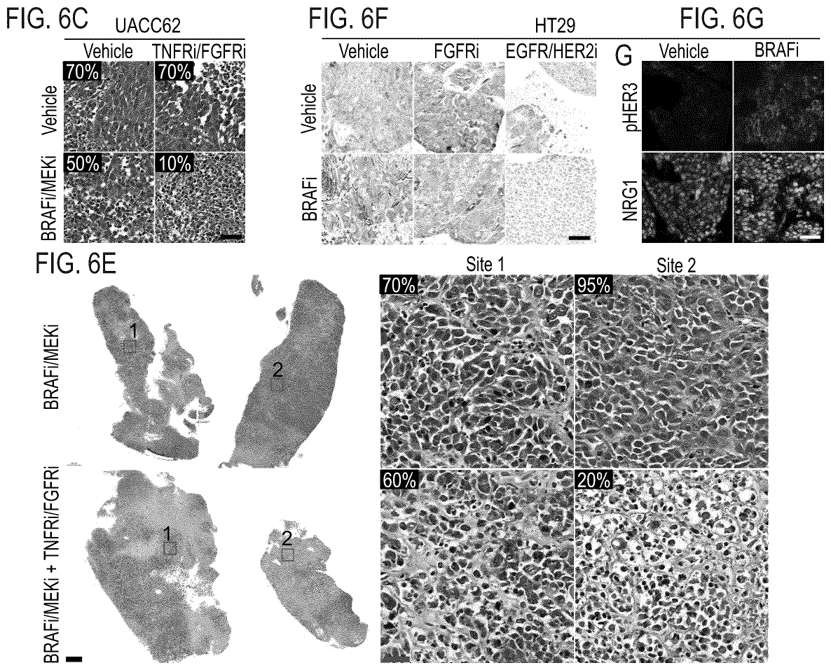

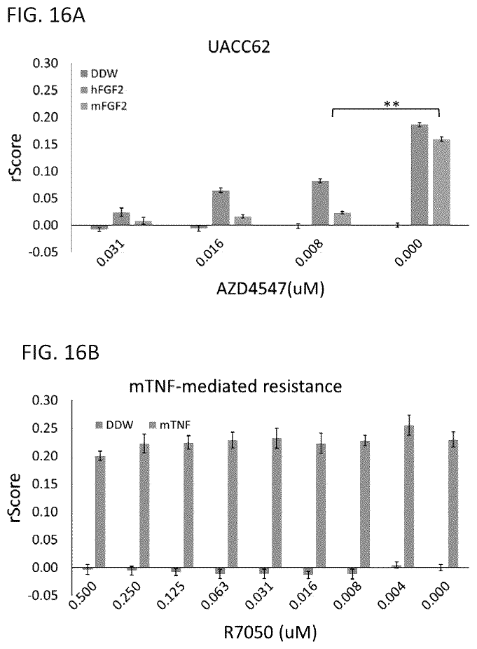

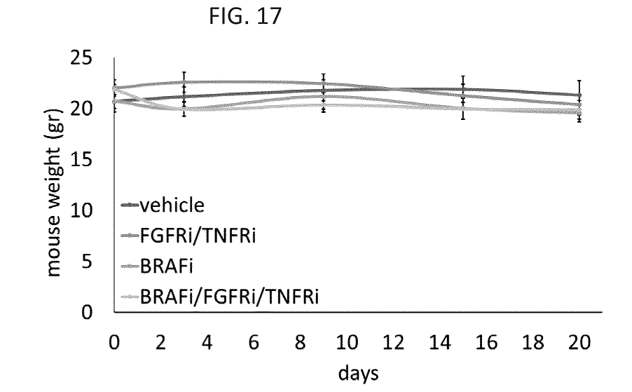

The human melanoma UACC62 BRAF (V600E)-mutated cell line was injected subcutaneously into nude mice. Established tumors were resected, and their sensitivity to BRAFi with or without co-targeting potential mechanisms of resistance was tested ex-vivo. In accordance with previous reports, BRAF/MEK inhibition had only a partial effect on cell viability (FIG. 6A). Co-targeting potential mechanisms of resistance to BRAF inhibition demonstrated that inhibition of the TNF and FGF pathways, in addition to BRAF/MEK inhibition, significantly reduced cell viability relative to BRAF/MEK inhibition only (FIG. 6A). This result coincides with the results of the in-vitro screen, demonstrating that out of 297 factors tested, members of the FGF and TNF pathways (e.g., FGF2, TNF, LTA, IL1A and CCL4) conferred the strongest effect on resistance to BRAF/MEK inhibition (FIG. 6B). Note that both mouse FGF2 and mouse TNFa confer resistance to BRAFi in the human UACC62 cell line (FIGS. 16A-B), suggesting that these mouse factors are relevant to the FGF- and TNF-mediated mechanism of resistance observed ex vivo. A large variability was observed in the response to some of the treatments. For example, treatment with BRAF/MEKi exhibited a broad spectrum of response ranging between 20%-100% viability in tumors from different mice. While the addition of FGFR/TNFRi to BRAF/MEKi resulted in a significant decrease in the viability of cancer cells, responses ranged from 10%-40% viability in tumors from different mice (FIGS. 6A-C). These observations further emphasize the need to tailor tumor-specific therapy, even in tumors with similar genetic background. As predicted by the ex vivo model, the effect of inhibiting the FGF/TNF pathways on the response to BRAF inhibition was validated in-vivo (FIG. 6D) without causing severe clinical side effects (FIG. 17).

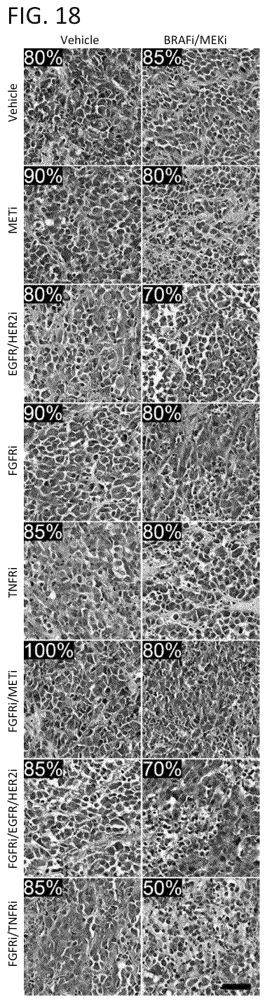

Finally, a freshly resected tumor biopsy from a 32 year old male with BRAF (V600E)-mutated melanoma that was clinically resistant to BRAF/MEK inhibition was obtained. The response of the cancer cells in this tumor to BRAF/MEK inhibition was tested ex-vivo with or without targeting potential mechanisms of resistance (FIG. 18). While the ex-vivo model clearly demonstrated that the tumor is resistant to BRAF/MEK inhibition, it also demonstrated that co-targeting the FGF/TNF pathways significantly decreased the viability of the cancer cells (FIG. 6E). Despite this significant response to the combination treatment, we still observed high intra-lesion variability between different sites in the tumor biopsy (FIG. 6E).

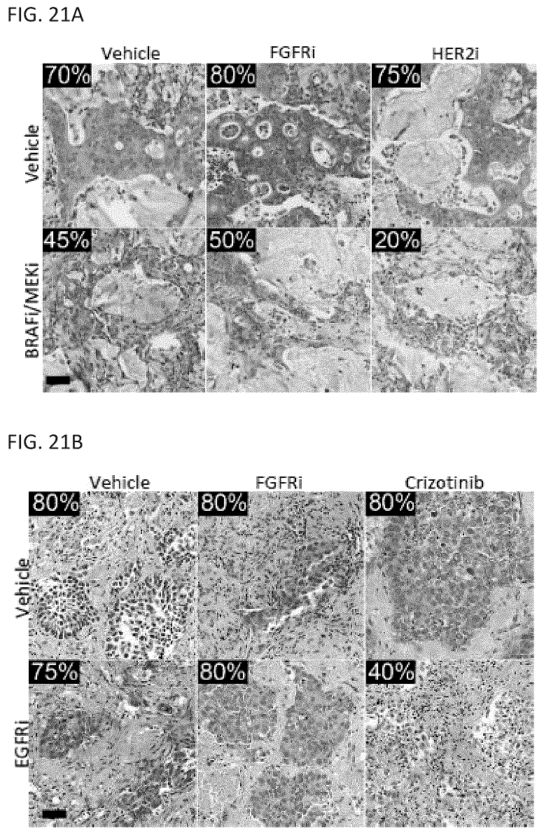

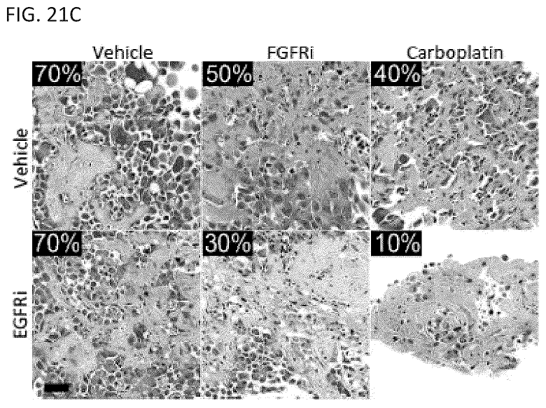

To test an alternative model of BRAF (V600E)-mutated tumors, a high-resolution endoscopic system (47) was used to generate orthotopic xenograft colorectal tumors, by injecting HT-29 BRAF (V600E)-mutated colorectal cancer cells into the colonic submucosa of mice. It has been previously shown that EGFR and HER2/3 heterodimer signaling may drive resistance to BRAF inhibition in BRAF-mutated colorectal adenocarcinoma (36,48). Consistent with previous reports (37), the EVOC model of the HT-29 colon tumors demonstrated that treatment with vemurafenib did not inhibit downstream pERK signaling (FIG. 6F). By contrast, combining BRAFi with the EGFR/HER2 inhibitor lapatinib, completely blocked pERK signaling (FIG. 6F). High levels of neuregulin-1 and hyperactivation of its receptor HER3 in response to vemurafenib were both detected ex vivo, mirroring their reported presence in human colorectal tumors (49) (FIG. 6G). Finally, a freshly resected tumor biopsy from a BRAF (V600E)-mutated colorectal patient was obtained (FIG. 21A) and the response of the cancer cells in this tumor to BRAF/MEK inhibition was tested ex-vivo with or without targeting potential mechanisms of resistance. In accordance with the EVOC model of HT-29 (FIG. 6F), while treatment with BRAF/MEK inhibitors partially reduced the cancer cell viability, co-targeting BRAF/MEK and the known EGFR/HER2 μmediated innate resistance mechanism resulted in a synergistic killing effect. Of note, co-targeting irrelevant innate resistance mechanism (FGFRi) did not improve the response to treatment.

### EGFR-Mutated Cancer Models

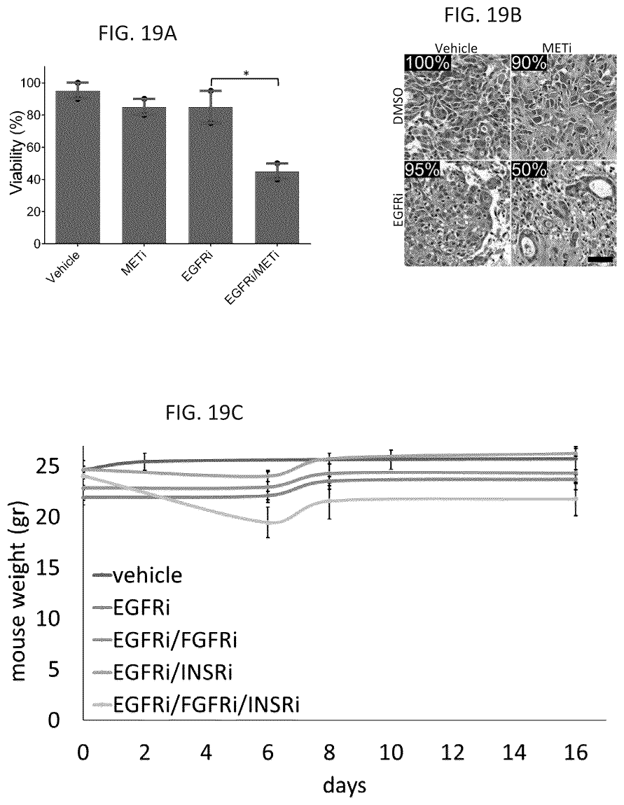

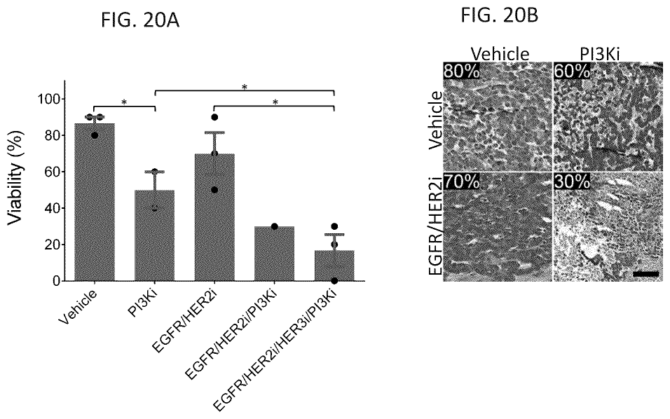

EVOCs were generated from xenograft tumors of a HCC4006 NSCLC cell line that was shown to have a moderate level of pMET, which may drive resistance to EGFR inhibition (50). Indeed, EVOC of HCC4006 xenograft tumors demonstrated that the addition of the MET inhibitor, crizotinib, reduced innate resistance to the EGFRi erlotinib (FIGS. 19A-B). To show the potential of co-targeting MET-mediated mechanisms of innate resistance in human NSCLC, a biopsy from a treatment-naïve, 62 years old male patient was obtained (FIG. 21B). While treatment with the third generation EGFRi osimertinib or its combination with FGFRi did not affect cancer cell viability, the combination of osimertinib with the METi, crizotinib exhibited a synergistic killing effect. This EVOC experiment thus suggests that treating this patient with a combination of EGFRi and METi may result in a better response.

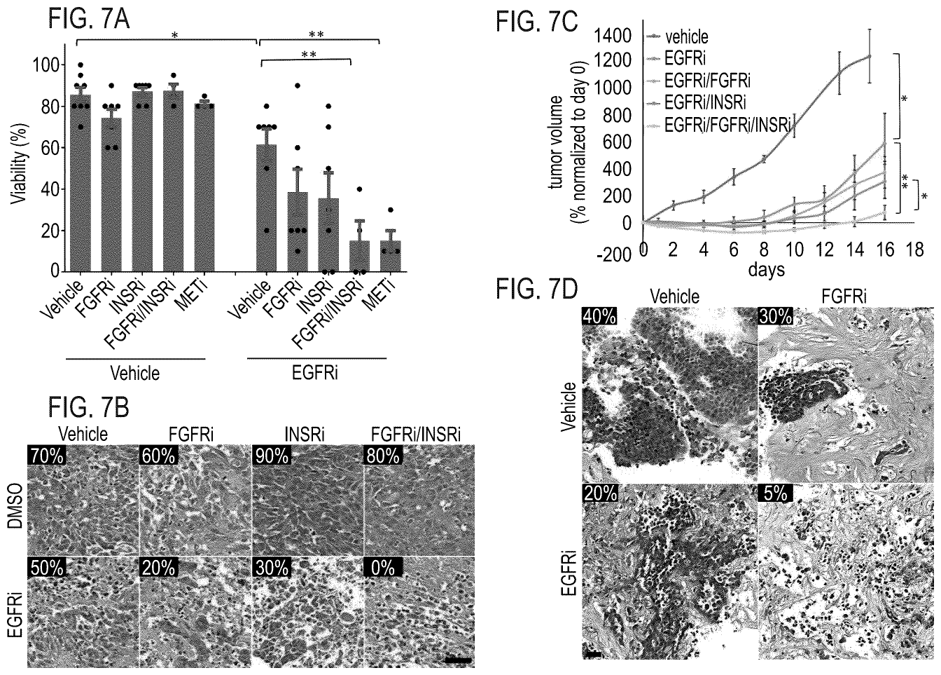

Next, the human NSCLC cell line H1975 was injected into the flank of nude mice. This cell line has an EGFR L858R activating mutation, as well as the T790M gatekeeper mutation that confers resistance to first-generation EGFR inhibitors. Established tumors were resected and their sensitivity to the second generation EGFR inhibitor, afatinib, with or without co-targeting potential mechanisms of resistance to EGFRi (FIG. 1E), was tested ex vivo. ERBB2 is one of the potential mechanisms of resistance to EGFRi, but it was not targeted with a specific drug because afatinib also blocks this receptor. Treatment with afatinib decreased the viability of cancer cells by only 28% on average, but the addition of FGFRi/INSRi or METi significantly reduced cell viability (FIGS. 7A-B). Testing the in vivo effect of adding FGFRi/INSRi to afatinib mirrored the EVOC results, demonstrating a partial effect of afatinib and the advantage of co-targeting the FGF and insulin receptors, without causing severe clinical side effects (FIGS. 7C and 19C).

To show the potential of co-targeting mechanisms of innate resistance in human NSCLC, a biopsy from an EGFR-mutated adenocarcinoma lung tumor of a non-smoker, treatment-naïve, 61 years old female patient was obtained. Using EVOC it was found that the addition of FGFRi to the EGFRi gefitinib significantly reduced cancer cells viability (FIG. 7D) suggesting that treating this patient with a combination of EGFRi and FGFRi may have resulted in a better response as compared to single treatment with EGFRi. Interestingly, a combination of the third generation EGFRi, osimertinib and the FGFRi AZD4547, exhibited an improved response as compared to osimertinib alone in a biopsy from a NSCLC EGFR mutated female patient who became refractory to osimertinib (FIG. 21C). Cancer cell viability following a combined treatment with FGFRi/EGFRi was considerably reduced as compared to treatment with EGFRi only, and was comparable to treatment with carboplatin. In clinical practice, such findings may justify favoring a combination treatment of targeted drugs over chemotherapy which is usually more toxic.

Taken together, the present inventors were interested in demonstrating that personalized anti-cancer treatment based on both tumor-specific genetic makeup and tumor specific innate resistance mechanisms may improve response to treatment. To this end, the landscape of innate resistance mechanisms in multiple human cell lines of several cancer types were characterized. However, the results also demonstrated that prioritization of the relevant patient-specific innate resistance mechanisms is challenging due to multiple variables. To address these obstacles, the present inventors proposed ex-vivo organ culture (EVOC) as a functional approach to test drug combinations which co-target the potential innate resistance mechanisms. Indeed, EVOCs from several mice cancer xenograft models as well as from human fresh biopsies were able to prioritize drug combinations which co-target both the driving mutation and the relevant innate resistance mechanisms. Thus, coupling knowledge of potential mechanisms of innate drug resistance with EVOC technology can be used to prioritize co-targeting of these mechanisms in a clinically relevant time scale, leading to better response to anti-cancer therapies.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Morash M, Mitchell H, Beltran H, Elemento O, Pathak J. The Role of Next-Generation Sequencing in Precision Medicine: A Review of Outcomes in Oncology. J Pers Med [Internet]. 2018 [cited 2020 Sep. 17]; 8. Available from:
   www(dot)ncbi(dot)nlm(dot)nih(dot)gov/pmc/articles/PMC6164147/
2. Bivona T G, Doebele R C. A framework for understanding and targeting residual disease in oncogene-driven solid cancers. Nat Med. 2016; 22:472-8.
3. Zahreddine H, Borden KLB. Mechanisms and insights into drug resistance in cancer. Front Pharmacol [Internet]. 2013 [cited 2020 Sep. 17]; 4. Available from:
   www(dot)ncbi(dot)nlm(dot)nih(dot)gov/pmc/articles/PMC3596793/
4. Senthebane D A, Rowe A, Thomford N E, Shipanga H, Munro D, Al Mazeedi MAM, et al. The Role of Tumor Microenvironment in Chemoresistance: To Survive, Keep Your Enemies Closer. Int J Mol Sci [Internet]. 2017 [cited 2020 Sep. 17]; 18. Available from: www(dot)ncbi(dot)nlm(dot)nih(dot)gov/pmc/articles/PMC5536073/
5. Straussman R, Morikawa T, Shee K, Barzily-Rokni M, Qian Z R, Du J, et al. Tumour micro-environment elicits innate resistance to RAF inhibitors through HGF secretion. Nature. 2012; 487:500-4.
6. Gusenbauer S, Vlaicu P, Ullrich A. HGF induces novel EGFR functions involved in resistance formation to tyrosine kinase inhibitors. Oncogene. 2013; 32:3846-56.
7. Lito P, Pratilas C A, Joseph E W, Tadi M, Halilovic E, Zubrowski M, et al. Relief of profound feedback inhibition of mitogenic signaling by RAF inhibitors attenuates their activity in BRAFV600E melanomas. Cancer Cell. 2012; 22:668-82.
8. Harbinski F, Craig V J, Sanghavi S, Jeffery D, Liu L, Sheppard K A, et al. Rescue screens with secreted proteins reveal compensatory potential of receptor tyrosine kinases in driving cancer growth. Cancer Discov. 2012; 2:948-59.
9. Wilson T R, Fridlyand J, Yan Y, Penuel E, Burton L, Chan E, et al. Widespread potential for growth-factor-driven resistance to anticancer kinase inhibitors. Nature. 2012; 487:505-9.
10. Lee Y, Wang Y, James M, Jeong J H, You M. Inhibition of IGF1R signaling abrogates resistance to afatinib (BIBW2992) in EGFR T790M mutant lung cancer cells. Mol Carcinog. 2016; 55:991-1001.
11. Terai H, Soejima K, Yasuda H, Nakayama S, Hamamoto J, Arai D, et al. Activation of the FGF2-FGFR1 autocrine pathway: a novel mechanism of acquired resistance to gefitinib in NSCLC. Mol Cancer Res MCR. 2013; 11:759-67.
12. Hata A N, Niederst M J, Archibald H L, Gomez-Caraballo M, Siddiqui F M, Mulvey H E, et al. Tumor cells can follow distinct evolutionary paths to become resistant to epidermal growth factor receptor inhibition. Nat Med. 2016; 22:262-9.
13. Lim Z-F, Ma P C. Emerging insights of tumor heterogeneity and drug resistance mechanisms in lung cancer targeted therapy. J Hematol OncolJ Hematol Oncol [Internet]. 2019 [cited 2020 Sep. 17]; 12. Available from:
   www(dot)ncbi(dot)nlm(dot)nih(dot)gov/pmc/articles/PMC6902404/
14. Meijer T G, Naipal K A, Jager A, van Gent D C. Ex vivo tumor culture systems for functional drug testing and therapy response prediction. Future Sci O A. 2017; 3:FS0190.
15. Naipal KAT, Verkaik N S, Sinchez H, van Deurzen CHM, den Bakker M A, Hoeijmakers JHJ, et al. Tumor slice culture system to assess drug response of primary breast cancer. BMC Cancer. 2016; 16:78.
16. Pemovska T, Kontro M, Yadav B, Edgren H, Eldfors S, Szwajda A, et al. Individualized systems medicine strategy to tailor treatments for patients with chemorefractory acute myeloid leukemia. Cancer Discov. 2013; 3:1416-29.
17. Martin S Z, Wagner D C, Horner N, Horst D, Lang H, Tagscherer K E, et al. Ex vivo tissue slice culture system to measure drug-response rates of hepatic metastatic colorectal cancer. BMC Cancer. 2019; 19:1030.
18. Misra S, Moro C F, Del Chiaro M, Pouso S, Sebestyén A, Löhr M, et al. Ex vivo organotypic culture system of precision-cut slices of human pancreatic ductal adenocarcinoma. Sci Rep. 2019; 9:2133.
19. van de Merbel A F, van der Horst G, van der Mark M H, van Uhm JIM, van Gennep E J, Kloen P, et al. An ex vivo Tissue Culture Model for the Assessment of Individualized Drug Responses in Prostate and Bladder Cancer. Front Oncol. 2018; 8:400.
20. Chen Y, Zhang Y, Yin Y, Gao G, Li S, Jiang Y, et al. SPD—a web-based secreted protein database. Nucleic Acids Res. 2005; 33:D169-73.
21. Lu C-C, Chu P-Y, Hsia S-M, Wu C-H, Tung Y-T, Yen G-C. Insulin induction instigates cell proliferation and metastasis in human colorectal cancer cells. Int J Oncol. Spandidos Publications; 2017; 50:736-44.
22. Jeong H, Kim J, Lee Y, Seo J H, Hong S R, Kim A. Neuregulin-1 induces cancer stem cell characteristics in breast cancer cell lines. Oncol Rep. Spandidos Publications; 2014; 32:1218-24.
23. Li J, Ballim D, Rodriguez M, Cui R, Goding C R, Teng H, et al. The Anti-proliferative Function of the TGF-P1 Signaling Pathway Involves the Repression of the Oncogenic TBX2 by Its Homologue TBX3. J Biol Chem. 2014; 289:35633-43.
24. Sacchi M, Klapan I, Johnson J T, Whiteside T L. Antiproliferative Effects of Cytokines on Squamous Cell Carcinoma. Arch Otolaryngol Neck Surg. American Medical Association; 1991; 117:321-6.
25. Bliss C I. The Toxicity of Poisons Applied Jointlyl. Ann Appl Biol. 1939; 26:585-615.
26. Heynen G J, Fonfara A, Bernards R. Resistance to targeted cancer drugs through hepatocyte growth factor signaling. Cell Cycle. 2014; 13:3808-17.
27. Montero J C, Rodriguez-Barrueco R, Ocana A, Diaz-Rodriguez E, Esparis-Ogando A, Pandiella A. Neuregulins and Cancer. Clin Cancer Res. American Association for Cancer Research; 2008; 14:3237-41.
28. Zhou Y, Wu C, Lu G, Hu Z, Chen Q, Du X. FGF/FGFR signaling pathway involved resistance in various cancer types. J Cancer. 2020; 11:2000-7.
29. Xiang A C, Xie J, Zhang X J. Acetylcholinesterase in intestinal cell differentiation involves G₂/M cell cycle arrest. Cell Mol Life Sci CMLS. 2008; 65:1768-79.
30. Zhao Y, Wang X, Wang T, Hu X, Hui X, Yan M, et al. Acetylcholinesterase, a key prognostic predictor for hepatocellular carcinoma, suppresses cell growth and induces chemosensitization. Hepatol Baltim Md. 2011; 53:493-503.
31. Zhang X J, Yang L, Zhao Q, Caen J P, He H Y, Jin Q H, et al. Induction of acetylcholinesterase expression during apoptosis in various cell types. Cell Death Differ. 2002; 9:790-800.
32. Xu H, Shen Z, Xiao J, Yang Y, Huang W, Zhou Z, et al. Acetylcholinesterase overexpression mediated by oncolytic adenovirus exhibited potent anti-tumor effect. BMC Cancer. 2014; 14:668.
33. Li C Y, Wood D K, Huang J H, Bhatia S N. Flow-based pipeline for systematic modulation and analysis of 3D tumor microenvironments. Lab Chip. 2013; 13:1969-78.
34. Konieczkowski D J, Johannessen C M, Abudayyeh O, Kim J W, Cooper Z A, Piris A, et al. A melanoma cell state distinction influences sensitivity to MAPK pathway inhibitors. Cancer Discov. 2014; 4:816-27.
35. Bottoni A, Vignali C, Piccin D, Tagliati F, Luchin A, Zatelli M C, et al. Proteasomes and RARS modulate AIMP1/EMAP II secretion in human cancer cell lines. J Cell Physiol. 2007; 212:293-7.
36. Prahallad A, Sun C, Huang S, Di Nicolantonio F, Salazar R, Zecchin D, et al.

Unresponsiveness of colon cancer to BRAF(V600E) inhibition through feedback activation of EGFR. Nature. 2012; 483:100-3.
37. Corcoran R B, Ebi H, Turke A B, Coffee E M, Nishino M, Cogdill A P, et al. EGFR-Mediated Reactivation of MAPK Signaling Contributes to Insensitivity of BRAF-Mutant Colorectal Cancers to RAF Inhibition with Vemurafenib. Cancer Discov. American Association for Cancer Research; 2012; 2:227-35.
38. Matsuda Y, Ueda J, Ishiwata T. Fibroblast Growth Factor Receptor 2: Expression, Roles, and Potential As a Novel Molecular Target for Colorectal Cancer [Internet]. Pathol. Res. Int. Hindawi; 2012 [cited 2020 Sep. 17]. page e574768. Available from: www(dot)hindawi(dot)com/journals/pri/2012/574768/
39. Jones J T, Akita R W, Sliwkowski M X. Binding specificities and affinities of egf domains for ErbB receptors. FEBS Lett. 1999; 447:227-31.
40. Tepper S R, Zuo Z, Khattri A, HeB J, Seiwert T Y. Growth factor expression mediates resistance to EGFR inhibitors in head and neck squamous cell carcinomas. Oral Oncol. 2016; 56:62-70.
41. Metzner T, Bedeir A, Held G, Peter-Vörösmarty B, Ghassemi S, Heinzle C, et al. Fibroblast Growth Factor Receptors as Therapeutic Targets in Human Melanoma: Synergism with BRAF Inhibition. J Invest Dermatol. 2011; 131:2087-95.
42. Zhang Z, Coomans C, David G. Membrane Heparan Sulfate Proteoglycan-supported FGF2-FGFR1 Signaling EVIDENCE IN SUPPORT OF THE “COOPERATIVE END STRUCTURES” MODEL. J Biol Chem. 2001; 276:41921-9.
43. Amaria R N, Prieto P A, Tetzlaff M T, Reuben A, Andrews M C, Ross M I, et al. Neoadjuvant plus adjuvant dabrafenib and trametinib versus standard of care in patients with high-risk, surgically resectable melanoma: a single-centre, open-label, randomised, phase 2 trial. Lancet Oncol. 2018; 19:181-93.
44. Kwong L N, Boland G M, Frederick D T, Helms T L, Akid A T, Miller J P, et al. Co-clinical assessment identifies patterns of BRAF inhibitor resistance in melanoma. J Clin Invest. American Society for Clinical Investigation; 2015; 125:1459-70.
45. Corso S, Giordano S. Cell-autonomous and non-cell-autonomous mechanisms of HGF/MET-driven resistance to targeted therapies: from basic research to a clinical perspective. Cancer Discov. 2013; 3:978-92.
46. Rexer B N, Arteaga C L. Intrinsic and Acquired Resistance to HER2-Targeted Therapies in HER2 Gene-Amplified Breast Cancer: Mechanisms and Clinical Implications. Crit Rev Oncog. 2012; 17:1-16.
47. Zigmond E, Halpern Z, Elinav E, Brazowski E, Jung S, Varol C. Utilization of murine colonoscopy for orthotopic implantation of colorectal cancer. PloS One. 2011; 6:e28858.
48. Herr R, Halbach S, Heizmann M, Busch H, Boerries M, Brummer T. BRAF inhibition upregulates a variety of receptor tyrosine kinases and their downstream effector Gab2 in colorectal cancer cell lines. Oncogene. 2018; 37:1576-93.
49. Stahler A, Heinemann V, Neumann J, Crispin A, Schalhorn A, Stintzing S, et al. Prevalence and influence on outcome of HER2/neu, HER3 and NRG1 expression in patients with metastatic colorectal cancer. Anticancer Drugs. 2017; 28:717-22.
50. Kubo T, Yamamoto H, Lockwood W W, Valencia I, Soh J, Peyton M, et al. MET gene amplification or EGFR mutation activate MET in lung cancers untreated with EGFR tyrosine kinase inhibitors. Int J Cancer J Int Cancer. 2009; 124:1778-84.
51. Cremolini C, Loupakis F, Antoniotti C, Lonardi S, Masi G, Salvatore L, et al. Early tumor shrinkage and depth of response predict long-term outcome in metastatic colorectal cancer patients treated with first-line chemotherapy plus bevacizumab: results from phase III TRIBE trial by the Gruppo Oncologico del Nord Ovest. Ann Oncol Off J Eur Soc Med Oncol. 2015; 26:1188-94.
52. Liu Y-T, Zhang K, Li C-C, Hu X-S, Jiang J, Hao X-Z, et al. Depth of Response was Associated with Progression-Free Survival in Patients with Advanced Non-small Cell Lung Cancer treated with EGFR-TKI. J Cancer. 2019; 10:5108-13.
53. Spigel D R, Ervin T J, Ramlau R A, Daniel D B, Goldschmidt J H, Blumenschein G R, et al. Randomized Phase II Trial of Onartuzumab in Combination With Erlotinib in Patients With Advanced Non-Small-Cell Lung Cancer. J Clin Oncol. 2013; 31:4105-14.
54. McCoach C E, Blumenthal G M, Zhang L, Myers A, Tang S, Sridhara R, et al. Exploratory analysis of the association of depth of response and survival in patients with metastatic non-small-cell lung cancer treated with a targeted therapy or immunotherapy. Ann Oncol. 2017; 28:2707-14.
55. Lewis K D, Larkin J, Ribas A, Flaherty K T, McArthur G A, Ascierto P A, et al. Impact of depth of response on survival in patients treated with cobimetinib ±vemurafenib: pooled analysis of BRIM-2, BRIM-3, BRIM-7 and coBRIM. Br J Cancer. Nature Publishing Group; 2019; 121:522-8.
56. Wagle N, Van Allen E M, Treacy D J, Frederick D T, Cooper Z A, Taylor-Weiner A, et al. MAP kinase pathway alterations in BRAF-mutant melanoma patients with acquired resistance to combined RAF/MEK inhibition. Cancer Discov. 2014; 4:61-8.

What is claimed is:

1. A method of selecting or determining therapeutic efficacy of a combination of agents for the treatment of cancer in a subject in need thereof, the method comprising:
(i) culturing a cancerous tissue of the subject in an ex-vivo organ culture (EVOC) in the presence of a combination of an anti-cancer agent and an additional agent, said additional agent does not have an anti-cancer effect as a single agent on said cancer as determined in an EVOC, however it is inhibiting expression and/or activity of a target conferring innate resistance to said anti-cancer agent or increasing expression and/or activity of a target conferring innate sensitivity to said anti-cancer agent; and
(ii) determining an anti-cancer effect of said combination on said tissue, wherein responsiveness of said tissue to said combination indicates said combination is efficacious for the treatment of said cancer in said subject.

2. A method of treating cancer in a subject in need thereof, the method comprising:
(a) selecting treatment or determining therapeutic efficacy of a combination of agents according to the method of claim 1; and
(b) administering to said subject a therapeutically effective amount of a combination demonstrating efficacy for the treatment of said cancer in said subject,
thereby treating the cancer in the subject.

3. The method of claim 1, wherein said responsiveness is increased responsiveness as compared to individual treatment with said anti-cancer agent, as determined by said EVOC system.

4. The method of claim 1, wherein said cancer is selected from the group consisting of melanoma, non-small cell lung cancer, ovarian cancer, breast cancer, pancreatic cancer, esophageal cancer, colorectal cancer and prostate cancer.

5. The method of claim 1, wherein said cancer is selected from the group consisting of melanoma, colorectal cancer, non-small cell lung cancer and esophageal cancer.

6. The method of claim 1, wherein cells of said cancer comprise a mutation associated with responsiveness to said anti-cancer agent.

7. The method of claim 1, wherein said anti-cancer agent is a target therapy agent.

8. The method of claim 1, wherein said anti-cancer agent is a cytotoxic agent.

9. The method of claim 1, wherein said target has been identified in an in-vitro screening assay prior to said (i).

10. The method of claim 1, wherein said target is a secreted factor or protein.

11. The method of claim 10, wherein cells of said cancer express a receptor of said target.

12. The method of claim 10, wherein said additional agent binds a receptor of said target.

13. The method of claim 1, wherein said target conferring innate resistance to said anti-cancer agent is selected from the group of targets listed in Table 3.

14. The method of claim 1, wherein said target conferring innate resistance to said anti-cancer agent is selected from the group consisting of, epigen (EPGN), soluble epidermal growth factor receptor (EGFR), endothelial-monocyte activating polypeptide II (EMAPII), matrix metallopeptidase 7 (MMP7), neurotrophin4 (NTF4), lymphotoxin alpha (LTA), TNF superfamily member 14 (TNFSF14), bone morphogenetic protein 10 (BMP10), ciliary neurotrophic factor (CNTF), C-C motif chemokine ligand 1 (CCL1) and folate receptor beta (FOLR2).

15. The method of claim 1, wherein said anti-cancer agent and said target conferring innate resistance to said anti-cancer agent are selected from the group of combinations listed in Table 4A.

16. The method of claim 1, wherein:
(i) said cancer is a BRAF mutated melanoma cancer, said anti-cancer agent is a BRAF/MEK inhibitor and said target conferring innate resistance to said anti-cancer agent is selected from the group consisting of TGFA, HBEGF, NRG1b, HGF, FGF2, FGF9, EMAPII, FGF4, FGF6, FGF18, FGF7, LTA, TNF, IL1A, TGFB1, TGFB2, TGFB3 and OSM;
(ii) said cancer is a BRAF mutated melanoma cancer, said anti-cancer agent is a BRAF/MEK inhibitor and said additional agent is a MET inhibitor, EGFR inhibitor, HER2 inhibitor, TGFBR inhibitor, gp130 inhibitor, FGFR inihibitor and/or TNFR inhibitor;
(iii) said cancer is an EGFR mutated NSCLC cancer, said anti-cancer agent is a EGFR inhibitor and said target conferring innate resistance to said anti-cancer agent is selected from the group consisting of NRG1b, INS, HGF, FGF2, EMAPII and FGF4;
(iv) said cancer is an EGFR mutated NSCLC cancer, said anti-cancer agent is an EGFR inhibitor and said additional agent is a FGFR inhibitor, INSR inhibitor, FGFR inhibitor and/or MET inhibitor;
(v) said cancer is an EGFR and PIK3CA mutated esophageal cancer, said anti-cancer agent is a PI3K inhibitor and said target conferring innate resistance to said anti-cancer agent is selected from the group consisting of EGF, BTC, TGFA, HBEGF, EPGN, NRG1a and NRG1b; or
(vi) said cancer is an EGFR and PIK3CA mutated esophageal cancer, said anti-cancer agent is a PI3K inhibitor and said additional agent is a EGFR inhibitor, HER2 inhibitor, and/or HER3 inhibitor.

17. The method of claim 1, wherein said target conferring innate sensitivity to said anti-cancer drug is selected from the group of targets listed in Table 5.

18. The method of claim 1, wherein said target conferring innate sensitivity to said anti-cancer drug is selected from the group consisting of Transforming Growth Factor Beta 1-3 (TGFB1-3), Colony Stimulating Factor 2 (CSF2), Interleukin 10 (IL10), Platelet Derived Growth Factor Subunit B (PDGFB), Ephrin A5 (EFNA5), Soluble Epidermal Growth Factor Receptor (EGFR), Prokineticin 2 (PROK2), Relaxin 3 (RLN3), Peptide YY (PYY), acetylcholinesterase (ACHE), Amyloid P Component, Serum (APCS), Collagen Type IV Alpha 1 Chain (COL4A1) and Vitronectin (VTN).

19. The method of claim 1, wherein said anti-cancer agent and said target conferring innate sensitivity to said anti-cancer drug are selected from the group of combinations listed in Table 6A.

20. The method of claim 1, wherein:
(i) said cancer is a BRAF mutated melanoma cancer, said anti-cancer agent is a BRAF/MEK inhibitor and said target conferring innate sensitivity to said anti-cancer drug is selected from the group consisting of TGFB1, TGFB2, TGFB3, BMP2, CFS2,IL10, RLN3 and ACHE;
(ii) said cancer is an EGFR mutated NSCLC cancer or PDAC cancer, said anti-cancer agent is a mitosis inhibitor and said target conferring innate sensitivity to said anti-cancer drug is TGFB3 and/or BMP4;
(iii) said cancer is an ovarian cancer, said anti-cancer agent is an EGFR inhibitor and said target conferring innate sensitivity to said anti-cancer drug is TNFa; or
(iv) said cancer is a BRAF wild-type melanoma, said anti-cancer agent is an MDM2 inhibitor or a Hsp90 inhibitor and said target conferring innate sensitivity to said anti-cancer drug is APCS.
